SEQUENCE LISTING (36 sequences)

SEQ ID NO: 1 - DNA, 22 bp, synthetic construct
  source 1..22: mol_type = other DNA
caccattggcaatgagcggttc

SEQ ID NO: 2 - DNA, 22 bp, synthetic construct
  source 1..22: mol_type = other DNA
aggtctttgcggatgtccacgt

SEQ ID NO: 3 - DNA, 22 bp, synthetic construct
  source 1..22: mol_type = other DNA
gtcacagcatgatgcaggacca

SEQ ID NO: 4 - DNA, 22 bp, synthetic construct
  source 1..22: mol_type = other DNA
tctgcgagctggtcatggagtt

SEQ ID NO: 5 - DNA, 22 bp, synthetic construct
  source 1..22: mol_type = other DNA
ctccaacatcctgaacctcagc

SEQ ID NO: 6 - DNA, 23 bp, synthetic construct
  source 1..23: mol_type = other DNA
cgtcacaccattgctattcttcg

SEQ ID NO: 7 - DNA, 22 bp, synthetic construct
  source 1..22: mol_type = other DNA
cctgaagcagaagaggatcacc

SEQ ID NO: 8 - DNA, 22 bp, synthetic construct
  source 1..22: mol_type = other DNA
aaagcggcagatggtcgtttgg

SEQ ID NO: 9 - DNA, 22 bp, synthetic construct
  source 1..22: mol_type = other DNA
caaggaggagctgctcttcatg

SEQ ID NO: 10 - DNA, 22 bp, synthetic construct
  source 1..22: mol_type = other DNA
tggtgctctcctcaatctgctg

SEQ ID NO: 11 - DNA, 21 bp, synthetic construct
  source 1..21: mol_type = other DNA
cctataaatccacgccggttc

SEQ ID NO: 12 - DNA, 20 bp, synthetic construct
  source 1..20: mol_type = other DNA
aacgaggctgcctgaagtca

SEQ ID NO: 13 - DNA, 23 bp, synthetic construct
  source 1..23: mol_type = other DNA
tctcatttcgtgatggagactgc

SEQ ID NO: 14 - DNA, 21 bp, synthetic construct
  source 1..21: mol_type = other DNA
gtgtcccgttcttgcatttgc

SEQ ID NO: 15 - DNA, 19 bp, synthetic construct
  source 1..19: mol_type = other DNA
ggccagatcctgtccaagc

SEQ ID NO: 16 - DNA, 21 bp, synthetic construct
  source 1..21: mol_type = other DNA
gtgggtttccaccattagcac

SEQ ID NO: 17 - DNA, 18 bp, synthetic construct
  source 1..18: mol_type = other DNA
cagccatacagcaaatcc

SEQ ID NO: 18 - DNA, 18 bp, synthetic construct
  source 1..18: mol_type = other DNA
atcctgtccgggtacaat

SEQ ID NO: 19 - DNA, 21 bp, synthetic construct
  source 1..21: mol_type = other DNA
ggacaagcagtgaccatcaag

SEQ ID NO: 20 - DNA, 23 bp, synthetic construct
  source 1..23: mol_type = other DNA
cccagaattaccaagtgagtcct

SEQ ID NO: 21 - DNA, 21 bp, synthetic construct
  source 1..21: mol_type = other DNA
aaggtgaaggtcggagtcaac

SEQ ID NO: 22 - DNA, 22 bp, synthetic construct
  source 1..22: mol_type = other DNA
ggggtcattgatggcaacaata

SEQ ID NO: 23 - DNA, 24 bp, synthetic construct
  source 1..24: mol_type = other DNA
ggtctttgacgctcggaactgtag

SEQ ID NO: 24 - DNA, 24 bp, synthetic construct
  source 1..24: mol_type = other DNA
cacaactgggtgaactccaaggtg

SEQ ID NO: 25 - DNA, 20 bp, synthetic construct
  source 1..20: mol_type = other DNA
gtgaagaacccacggtctgt

SEQ ID NO: 26 - DNA, 20 bp, synthetic construct
  source 1..20: mol_type = other DNA
ctggttgtcaggggagtgtt

SEQ ID NO: 27 - DNA, 22 bp, synthetic construct
  source 1..22: mol_type = other DNA
cctactcgaagacttacccagt

SEQ ID NO: 28 - DNA, 21 bp, synthetic construct
  source 1..21: mol_type = other DNA
gcattggggtgaatgatagca

SEQ ID NO: 29 - DNA, 20 bp, synthetic construct
  source 1..20: mol_type = other DNA
tgacatcactgcagccatac

SEQ ID NO: 30 - DNA, 20 bp, synthetic construct
  source 1..20: mol_type = other DNA
tgggacagatctcggttttg

SEQ ID NO: 31 - DNA, 22 bp, synthetic construct
  source 1..22: mol_type = other DNA
cagccactttccaatttcactg

SEQ ID NO: 32 - DNA, 23 bp, synthetic construct
  source 1..23: mol_type = other DNA
gctggacatactccttcacctta

SEQ ID NO: 33 - DNA, 22 bp, synthetic construct
  source 1..22: mol_type = other DNA
aggtcagtcagcacttggcatt

SEQ ID NO: 34 - DNA, 21 bp, synthetic construct
  source 1..21: mol_type = other DNA
tcctggtggacctacctgctt

SEQ ID NO: 35 - DNA, 19 bp, synthetic construct
  source 1..19: mol_type = other DNA
gctggtgaaaaggacctct

SEQ ID NO: 36 - DNA, 20 bp, synthetic construct
  source 1..20: mol_type = other DNA
cacaggactagaacacctgc

(19) United States
(12) Patent Application Publication
(10) Pub. No.: US 2026/0315542 A1
(43) Pub. Date: Oct. 15, 2026

(54) MUSE CELLS FOR USE FOR TREATING RADIATION-INDUCED GASTRO-INTESTINAL SYNDROME

(71) Applicants: COMMISSARIAT A L'ENERGIE ATOMIQUE ET AUX ENERGIES ALTERNATIVES, PARIS (FR); UNIVERSITE PARIS-SACLAY, GIF-SUR-YVETTE (FR); INSTITUT NATIONAL DE LA SANTE ET DE LA RECHERCHE MEDICALE, PARIS (FR)

(72) Inventors: Nathalie GAULT, FONTENAY AUX ROSES (FR); Vanessa PETIT, FONTENAY AUX ROSES (FR); Paul-Henri ROMEO, FONTENAY AUX ROSES (FR); Stephanie MORENO, FONTENAY AUX ROSES (FR); Honorine DUSHIME, VENISSIEUX (FR)

(21) Appl. No.: 19/473,920

(22) PCT Filed: Apr. 15, 2024
(86) PCT No.: PCT/EP2024/060148

§ 371 Date: Oct. 9, 2025

(30) Foreign Application Priority Data
Apr. 14, 2023 (EP) 23305570.6

(51) Int. Cl. A61K 35/51; A61K 9/00; A61K 35/545; A61P 1/00

(52) U.S. Cl. CPC A61K 35/51; A61K 9/0019; A61K 35/545; A61P 1/00

(57) ABSTRACT

The present invention concerns novel Multilineage-differentiating stress enduring (Muse) cells, for use for treating a pathology selected from radiation-induced pathologies and pathologies of the intestine (preferably radiation-induced gastrointestinal syndrome (CIS)), wherein the Muse cells are SSEA-3⁺CD105⁺. The present invention also relates to compositions comprising Muse cells for use for treating a pathology selected from radiation-induced pathologies and pathologies of the intestine (preferably radiation-induced gastrointestinal syndrome (CIS)). The present invention also provides methods for treating a pathology selected from radiation-induced pathologies and pathologies of the intestine (preferably radiation-induced gastrointestinal syndrome (CIS)), comprising the administration of a therapeutically effective amount of Muse cells to a subject in need thereof.

### TECHNICAL FIELD OF THE INVENTION

The present invention relates to the fields of cell biology and cell therapy, more specifically radiation-induced pathologies and pathologies of the intestine. In particular, the invention concerns novel Multilineage-differentiating stress enduring (Muse) cells, in particular human Muse cells, for use for treating a pathology selected from radiation-induced pathologies and pathologies of the intestine, wherein the Muse cells are SSEA-3⁺CD105⁺.

The present invention also concerns compositions comprising Muse cells for use for treating a pathology selected from radiation-induced pathologies and pathologies of the intestine. The present invention also provides methods for treating a pathology selected from radiation-induced pathologies and pathologies of the intestine, comprising the administration of a therapeutically effective amount of Muse cells to a subject in need thereof.

### BACKGROUND ART

The intestine is one of the most radiosensitive organs in the body and is the most radiosensitive tissue of the intra-abdominal area, as it is a renewable tissue, especially intestinal mucosa which is replaced every 3-5 days [1]. The intestinal mucosa is divided into three distinct layers including Lamina epithelialis (LE), Lamina propria (LP), rich in vascular, lymphatic network and leucocytes, and muscularis mucosae. The central component of intestinal mucosal barrier is the LE, organized in two connected structures called villi and crypts. Villi are made up of mature functional cells including mostly absorptive enterocytes connected by tight junctions [2]. Crypts contain two populations of intestinal stem cells (ISC), identified by the markers Lgr5 or Bmi-1, and transit amplifying cells. Lgr5⁺ISC are mitotically active and ensure the continual renewal of the epithelium cells, whereas Bmi-1⁺ISC are quiescent and their function during homeostasis or injury is still debate [3,4]. Regulation of ISC behaviours in intestinal homeostasis occurs within a microenvironment confined to the crypt base, known as the stem cell niche [5]. The microenvironment includes multiple cell types such as Paneth cells, closely tied to Lgr5⁺ISC and monocytes/macrophages and secretes cell-associated ligands, chemokines, soluble growth factors and cytokines [6,7]. Under intestinal injury, frequently accompanied by inflammation, Paneth cells [8] and macrophages [9] promote the repair of intestinal tissue by regulating ISC function. Radiation-induced gastrointestinal syndrome (GIS) is a lethal disease occurring after therapeutic or accidental exposure to high doses of whole-body irradiation (IR) or significant whole-abdominal IR [10]. Intestine symptoms occur few days after exposure and their severity depends on the dose of irradiation. They include weight loss and diarrhoea, leading to dehydration and electrolyte loss, and to an increased susceptibility to infection due to the intestinal mucosal barrier breakdown, facilitating the entry of bacteria into the bloodstream and leading ultimately to the death by sepsis.

The intestinal clinical signs and symptoms of the GIS result from the lack of replacement of mature functional cells at the surface of the villi, because stem and proliferating cells of the crypts are irreversibly damaged by radiations and die by apoptosis or mitotic death. In addition, IR induces microvascular damages due to endothelial cell apoptosis and an important inflammatory response in the intestine, characterized by inflammatory cell infiltration and an overproduction of pro-inflammatory mediators contributing to perpetuate damage cells [1].

Mesenchymal Stem cells (MSC)-based therapies have been previously shown as a pre-clinical approach to stimulate epithelial stem cells to repair radio-induced intestinal injury [37]. Indeed, previous studies have shown that injection of MSC into irradiated mice resulted in an improved regeneration of intestinal or colonic tissues increasing the animal survival. Their effectiveness is essentially based on their secretion of growth factors with anti-inflammatory and pro-angiogenic effects but requires 1 to 5 million injected MSC per mouse within 2-24 hours after IR. Thus, in vitro amplification of MSC by weeks of culture limits their use for immediate treatment of emergency conditions. In MSC clinical trials, the dosages of MSC are typically described in cells/kg body weight (0.5-12×10⁶ cells/kg through single or multiple doses) [38]. Extensive expansion by in vitro cultures, cryopreservation and thawing, and long-term storage of MSC may represent serious constraints and cost. Moreover, these processes may decrease the therapeutic efficacy of MSC [39,40]. Thus, several priming approaches or genetically modified MSC have been proposed to improve the migration, homing, survival and function of MSC [41]. More recently, Bensemmane et al., showed that injection of stromal vascular fraction from adipose tissue which takes only 4 hours for preparation mitigates the GIS [26]. Nevertheless, this fraction which contains heterogeneous cell populations requires the injection of 2 million cells per mouse.

The discovery of Multilineage-differentiating stress-enduring (Muse) has renewed interest in stem cell-based therapy applications.

Muse cells are endogenous pluripotent-like stem cells collectable through the pluripotent stem cell surface marker Stage-Specific Embryonic Antigen 3 (SSEA-3) from multiple sources including the bone marrow, peripheral blood, adipose tissue and umbilical cord [12]. Muse cells can migrate into the injured tissues where they exert pleiotropic effects including anti-inflammatory actions, vascular protection and antiapoptotic responses [13]. Another important and unique feature is that allogeneic-Muse cells escape host immune-rejection after intravenous administration and survive in the host tissue as differentiated cells for over 6 months without immunosuppressive treatment [14]. Based on the safety and efficacity already demonstrated in preclinical studies, clinical trials using intravenous infusion of Muse cells are currently in progress for the treatment of human diseases such as acute myocardial infarction, ischemic stroke, spinal cord injuries and amyotrophic lateral sclerosis [15,16]. Currently, no data describe Muse-based therapy for radiation-induced pathologies (such as in radioinduced GIS) or pathologies of the intestine.

To date, there is no effective medical treatment for radiation-induced pathologies, including GIS [11]. The same applies to most pathologies of the intestine, where the treatments that are available focus mainly on alleviating symptoms, rather than treating the causes of the pathology. Thus, there is a compelling need for effective and rapid countermeasures.

### SUMMARY OF THE INVENTION

The present invention fulfils this need. Indeed, the present Inventors have designed a novel treatment of radiation-induced pathologies and pathologies of the intestine. More specifically, the Inventors demonstrate for the first time that the herein-described population of Multilineage-differentiating stress-enduring (Muse) cells is capable of rapidly and durably regenerating injured intestine tissue.

The data unexpectedly show that single injection of a small quantity of Muse cells after irradiation (IR), highly improved survival as a result of a rapid regeneration of intestinal epithelium with the rescue of the impaired epithelial barrier.

Therefore, the present invention provides an original, efficient, and easy therapeutic strategy for treating lethal radiation-induced pathologies as well as pathologies of the intestine.

The present invention thus relates to novel Multilineage-differentiating stress enduring (Muse) cells for use for treating a pathology selected from radiation-induced pathologies and pathologies of the intestine, wherein the Muse cells are SSEA-3⁺CD105⁺. In a preferred embodiment, the Muse cells are further characterized by a surface marker selected from: CD44⁺; CD73⁺; CD90⁺; CD45; and any combination thereof. The Muse cells are preferably mammal Muse cells, more preferably human Muse cells.

The Muse cells may be obtained from an adult or embryonic tissue, such as bone marrow, peripheral blood, adipose tissue, umbilical cord, or any mixture thereof. Muse cells are preferably obtained from umbilical cord, more preferably from Wharton's jelly of umbilical cord. The present invention also concerns compositions comprising Muse cells for use for treating a pathology selected from radiation-induced pathologies and pathologies of the intestine.

The present invention also provides methods for treating a pathology selected from radiation-induced pathologies and pathologies of the intestine, comprising the administration of a therapeutically effective amount of Muse cells to a subject in need thereof.

### DETAILED DESCRIPTION OF THE INVENTION

In the context of the present invention, the Inventors surprisingly found that administering a population of cells, called Multilineage-differentiating stress-enduring (Muse) cells, significantly increases survival of irradiated mice. The Muse cells promote rapid and durable regeneration of injured tissues, especially injured intestine tissues, as supported by the experimental data. In particular, the Inventors demonstrate for the first time that a single injection of a small quantity of Muse cells (only 50,000 Muse cells) generates a beneficial stem cell microenvironment, favouring the reconstitution of intestinal barrier, leading ultimately to full recovery and survival following irradiation (IR).

The data obtained by the Inventors unexpectedly show that Muse cells migrate into irradiated tissues, such as irradiated small intestine and persist into the crypts. In small intestine of Muse-treated mice, an early enhanced secretion of IL-6 and MCP-1 cytokines was observed associated with (i) recruitment of monocytes/M2-like macrophages and (ii) proliferation of Paneth cells through activation of the IL-6/Stat3 pathway.

The data further highlight that Muse cells promote the regeneration of intestinal epithelium characterized by (i) a hyperproliferation of crypt cells, (ii) an increased expression of the tight junction protein ZO-1, and (iii) an increased expression of adherent protein EpCAM.

Through this breakthrough work, the Inventors thus provide for the first time an original, efficient, and easy therapeutic strategy for treating lethal radiation-induced pathologies as well as pathologies of the intestine.

### DEFINITIONS

Unless specifically defined herein, all technical and scientific terms used herein have the same meaning as commonly understood by a skilled artisan in chemistry, biochemistry, cellular biology, molecular biology, and medical sciences.

As used herein throughout the entire text, the terms “a” and “an” are used in the sense that they mean “at least one”, “at least a first”, “one or more” or “one or a plurality” of the referenced compounds or steps, unless the context dictates otherwise.

The term “and/or” wherever used herein includes the meaning of “and”, “or” and “all or any other combination of the elements connected by said term”.

The term “about” or “approximately” as used herein means within 10%, preferably within 8%, and more preferably within 5%, and more preferably within 3%, and more preferably within 1% of a given value or range.

As used herein, when used to define products, compositions, cells, uses and methods, the term “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) is open-ended and do not exclude additional, unrecited elements or method steps. Thus, a polypeptide “comprises” an amino acid sequence when the amino acid sequence might be part of the final (and/or whole) amino acid sequence of the polypeptide. Such a polypeptide can have up to several hundred additional amino acid residues (e.g., linker and antioxidant moiety as described herein). “Consisting of” means excluding any other components or steps “consisting essentially of” mean excluding other components or steps of any essential significance (however, other minor/insignificant components or steps are not excluded). In the present disclosure, the terms “comprising”, “consisting of” and “consisting essentially of” may be replaced with each other, if required.

The terms “obtained from”, “originating from”, “originate from”, or “of XXX origin” (wherein XXX is any source) are used to identify the original source of a component (e.g., polypeptide, nucleic acid molecule) but are not meant to limit the method by which the component is made which can be, for example, by chemical synthesis or recombinant means.

As used herein, the terms “biological sample” or “sample” refer to an entire organ or tissue or fluid (e.g. blood, serum, plasma, milk, etc.) of one or more subject(s), or cells or cell components thereof (e.g. organelles, nucleic acids, proteins, etc.), or a fraction of tissue, organ, fluid, or cell, or a homogenate, a lysate or a crude or purified extract prepared from an entire organ or tissue or fluid of one or more subject(s), or cells or cell components thereof, or a fraction of tissue, organ, fluid, or cell. In particular, a “biological sample” or “sample” may be any tissue or fluid which may contain Muse cells including, but not limited to, a bone marrow sample, a peripheral blood sample, an adipose tissue sample, an umbilical cord sample, a plasma sample, a serum sample, and any combination thereof.

As used herein, the terms “treat”, “treating”, “treatment” and the like mean the reduction, inhibition, amelioration, stabilization and/or disappearance of a disease (or an ailment, or a condition, or a pathology), of the causes of a disease, of the symptoms (or signs) of a disease, of the effects (or consequences, preferably adverse, deleterious effects/consequences, including damages and/or injuries) of a disease (e.g., radiation-induced pathologies and pathologies of the intestine, and/or symptoms associated therewith), or fighting the disease, or any combination thereof. It will be appreciated that, although not precluded, treating a disorder or condition does not require that the disorder, condition or symptoms associated therewith be completely eliminated. For examples, treating results in the reduction of at least one sign or symptom of the disease or condition. Treatment includes (but is not limited to) administration of a therapy, and may be performed either prophylactically, or subsequent or the initiation of a pathologic event. Treatment can require administration of a therapy (or drug) more than once.

More specifically, “treating a radiation-induced pathology or a pathology of the intestine” refers to fighting at least one radiation-induced pathology and/or at least one pathology of the intestine in a subject (including a human or animal organism). These terms also refer to reducing/inhibiting/ameliorating/stabilizing/making disappear, the symptoms/signs/damages/injuries associated with the radiation-induced pathology or the pathology of the intestine (e.g., tissue damages/injuries (such as intestine tissue damages/injuries), kidney failure, fever, etc.) in the treated organism, advantageously within a shorter period of time than expected without treatment.

“Radiation-induced pathology” herein means a pathology (or a disease, or an ailment, or a condition, or a syndrome) which is provoked, caused, amplified, maintained, and any combination thereof, by one-time, occasional, regular, prolonged or repeated exposure to radiation. Examples of radiation-induced pathologies include, but are not limited to, radiation-induced tissue lesion, and radiation-induced gastrointestinal syndrome (GIS). As used therein, “Radiation-induced gastrointestinal syndrome (GIS)” is a disease occurring after therapeutic or accidental exposure to high doses of radiation, such as whole-body irradiation (IR) or significant whole-abdominal IR.

As used herein, “radiation” is the emission or transmission of energy in the form of waves or particles through space or through a material medium. Radiation includes:
electromagnetic radiation, such as radio waves, microwaves, infrared, visible light, ultraviolet, x-rays, and gamma radiation (Y);
particle radiation, such as alpha radiation (a), beta radiation (B), proton radiation and neutron radiation (particles of non-zero rest energy);
acoustic radiation, such as ultrasound, sound, and seismic waves (dependent on a physical transmission medium);
gravitational radiation, that takes the form of gravitational waves, or ripples in the curvature of spacetime; and
any combination thereof.

“Pathology of the intestine” herein means a pathology (or a disease, or an ailment, or a condition) affecting (or damaging, or altering, or degrading, or occurring in, or any combination thereof) any part of the intestine tissue. Examples of pathologies of the intestine include, but are not limited to, colitis, ulcer of the intestine, inflammatory bowel disease (such as ulcerative colitis (UC), Crohn's disease (CD)), radiation-induced gastrointestinal syndrome (GIS), cancer of the intestine, and lesion of the intestine.

A “therapeutically effective amount” corresponds to the amount of each active entity (e.g., the amount/quantity of Muse cells) that is sufficient for producing a beneficial health result.

The terms “pharmaceutically acceptable” are herein intended to mean what is useful to the preparation of a pharmaceutical composition, and what is generally safe and non-toxic, for a pharmaceutical use.

As used herein, a “pharmaceutically acceptable vehicle” is intended to include any and all carriers, solvents, diluents, excipients, adjuvants, dispersion media, coatings, antibacterial and antifungal agents, and absorption delaying agents, and the like, compatible with administration in a subject and in particular in a human. For general guidance, appropriate carriers for use herein are well known the art (see for example the most current edition of Remington: The Science and Practice of Pharmacy, A. Gennaro, Lippincott, Williams& Wilkins).

The term “subject” or “individual” generally refers to a vertebrate organism for whom any of the product (e.g., Muse cells), and/or uses, and/or methods, disclosed herein, is needed or may be beneficial. Typically, the subject is an animal, preferably a mammal, particularly a mammal selected from the group consisting of domestic animals, farm animals, sport animals, and primates (human and non-human); including, but not limited to, human, ovine, bovine, canine, feline, swine, rodents, monkeys, (animals including, e.g., dog, cat, cattle, goat, pig, rabbit, sheep, rat, mouse, etc.). The terms “subject” and “patient” may be used interchangeably when referring to a human organism and covers male and female as well as a foetuses, new-born, infant, young adult, adult and elderly.

As used herein, “subject in need” refers to a subject that may suffer from, or that is susceptible to suffer from, or that is suspected of suffering from, or that has been diagnosed with, a radiation-induced pathology and/or a pathology of the intestine.

The terms “Multilineage-differentiating stress enduring cells” or “Muse cells” herein refer to endogenous pluripotent-like stem cells expressing the pluripotent stem cell surface marker Stage-Specific Embryonic Antigen 3 (SSEA-3), and the CD105 marker. Muse cells have been identified in mammals, more particularly in human, rodents (such as mice and rats), rabbits, goats and pigs. Muse cells reside in multiple sources including the bone marrow, peripheral blood, adipose tissue and umbilical cord [12]. Muse cells are thus collectable through the SSEA-3 marker (throughout mammal species), from a sample of any of these sources including bone marrow sample, peripheral blood sample, adipose tissue sample and umbilical cord sample.

“Cell surface marker” or “Cell surface molecule” herein means a molecule (a moiety) displayed at the surface of a cell. Cell surface markers are typically macromolecules that are embedded in or span the layer of cell membranes. Cell surface markers may be any type of macromolecules, including proteins (including polypeptides, peptides, and the like), carbohydrates, lipids, nucleic acids, any mixture thereof (e.g., glycoproteins), and any complex thereof (e.g., protein complexes). Cell surface markers include Cluster of differentiation molecules.

“Cluster of differentiation” (abbreviated as CD) is commonly known as a nomenclature (or protocol) used for the identification and investigation of cell surface molecules, providing targets for immunophenotyping of cells. A CD molecule is thus a cell surface molecule, named according to the CD protocol. In an organism, CD molecules have numerous functions. They often act as receptors or ligands, generally as part of a signal cascade (altering/modifying the behaviour of the cell).

A CD molecule (and more generally a cell surface marker) may be notably detected using well known technologies such as cell membrane staining using biotinylation or other equivalent techniques followed by immunoprecipitation with specific antibodies, flow cytometry, western blot, ELISA or ELISPOT, antibodies microarrays, or tissue microarrays coupled to immunohistochemistry. Other suitable techniques include FRET or BRET, single cell microscopic or histochemistry methods using single or multiple excitation wavelength and applying any of the adapted optical methods, such as electrochemical methods (voltammetry and amperometry techniques), atomic force microscopy, and radio frequency methods, e.g. multipolar resonance spectroscopy, confocal al and non-confocal, detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry), cell ELISA, radioisotopic, magnetic resonance imaging, analysis by polyacrylamide gel electrophoresis (SDS-PAGE); HPLC-Mass Spectroscopy; Liquid Chromatography/Mass Spectrometry/Mass Spectrometry (LC-MS/MS)).

Detection of CD molecules (and more generally of cell surface markers) is preferably performed by flow cytometry.

As used herein, “flow cytometry” or “FCM” is a technique used to detect and measure physical and chemical characteristics of a population of cells or particles, for example in a sample (including a biological sample obtained from a subject). FCM is a useful tool for simultaneously measuring multiple physical properties of individual particles (such as cells, biomarkers, proteins, protein complexes, etc.). Cells pass single-file through a laser beam. As each cell passes through the laser beam, the cytometer records how the cell or particle scatters incident laser light and emits fluorescence. Using a flow cytometric analysis protocol, one can perform a simultaneous analysis of surface molecules at the single-cell level. Advantageously, the use of fluorescent agents or fluorochromes linked or attached to an antibody or antiserum able to specifically recognize a molecule or particle (such as a cell surface molecule (e.g., SSEA-3, CD105, CD44, CD45, CD73, CD90, CD3, Ly6C, CD68, CD206, EpCAM, Ki67, CD24, etc., and any combination thereof)) attached to a cell or portion thereof, a biomarker, a protein, a protein complex, etc., enables the flow cytometer to sort the molecules/particles on the basis of size, granularity and fluorescent light. Thus, the amount of information obtained from a single sample can be further expanded by using multiple fluorescent reagents. The information gathered by the flow cytometer can be displayed as any combination of parameters selected by the skilled person. For example, the flow cytometer can be configured to provide information about the relative size (forward scatter or “FSC”), granularity or internal complexity (side scatter or “SSC”), and relative fluorescent intensity of the cell sample. The detection of cell surface molecules (including CD molecules), cell surface receptors, Muse cells, monocytes, neutrophils, other granulocytes, T cells, B cells, natural killer cells, other cells, and any combination thereof, is performed by an exclusion gating strategy by flow cytometry. This combination is particularly useful to detect, identify, quantify, and sort cells, such as Muse cells, monocytes, neutrophils, granulocytes, T cells, B cells, natural killer cells and other cells. The detection can be practiced with any antibody or antiserum detecting (or recognizing specifically) cell surface molecules, antigens, receptors, or any combination thereof, expressed (and preferably exposed at the cell surface) by Muse cells, monocytes, neutrophils, granulocytes, T cells, B cells, natural killer cells and other cells. Advantageously, the cells present in the biological sample to analyse/quantify/sort are contacted with labelled antibodies (preferably labelled with a fluorescent agent or a fluorochrome), each of which recognizing a specific cell surface molecule, antigen, receptor, or any combination thereof, expressed (and preferably exposed at the cell surface) by the Muse cells, monocytes, neutrophils, granulocytes, T cells, B cells, natural killer cells and other cells (e.g., SSEA-3, CD105, CD44, CD45, CD73, CD90, CD3, Ly6C, CD68, CD206, EpCAM, Ki67, CD24, etc., and any combination thereof). The sample is then analysed by flow cytometry.

The terms “Stage-specific embryonic antigen 3” or “SSEA-3” or “SSEA3” herein refer to a glycosphingolipid, composed of an oligosaccharide containing five carbohydrate units connected to a sphingolipid. SSEA3 plays a role in cell signalling.

By “CD105” or “cluster of differentiation 105” is designated a cell-surface glycoprotein part of the TGF beta receptor complex. CD105 is also known as Endoglin (ENG), END, FLJ41744, HHT1, ORW, or ORW1. The CD105 glycoprotein typically consists of a homodimer of 180 kDA stabilized by intermolecular disulphide bonds. It has a large extracellular domain of about 561 amino acids, a hydrophobic transmembrane domain and a short cytoplasmic tail domain composed of 45 amino acids. There are two isoforms of CD105 created by alternative splicing: the long isoform (L-endoglin) and the short isoform (S-endoglin) CD105 has an important role in angiogenesis. The amino acid sequences of human CD105 are well known in the art, and are for instance available under NCBI accession number NP_000109.1, or NP_001108225.1, or NP_001265067.1, or NP_001108225.1, or NP_001265067.1.

As used herein, “CD44” or “cluster of differentiation 44” is a cell-surface glycoprotein involved in cell-cell interactions, cell adhesion and migration. CD44 is also known as HCAM (homing cell adhesion molecule), Pgp-1 (phagocytic glycoprotein-1), Hermes antigen, lymphocyte homing receptor, ECM-III, and HUTCH-1. CD44 participates in a wide variety of cellular functions including lymphocyte activation, recirculation and homing, haematopoiesis, and tutor metastasis. CD44 is a receptor for hyaluronic acid and can also interact with other ligands, such as osteopontin, collagens, and matrix metalloproteinases (MMPs). CD44 function is controlled by its posttranslational modifications. The amino acid sequence of human CD44 is well known in the art, and is for instance available under NCBI accession number ACI46596.1 or NP_000601.3 or NP_001001389.1, or NP_001001390.1, or NP_001001391.1, or NP_001001392.1.

As used therein, “cluster of differentiation CD45” or “CD45 molecule/antigen” or “CD45” is a single chain integral membrane protein, comprising at least 5 isoforms, ranging from 180 to 220 kDa. They are generated by alternative splicing combinations of three exons (A, B, and C) of the genomic sequence. CD45 has been referred to as PTPRC, “protein tyrosine phosphatase receptor type C” (PTPRC), or leukocyte common antigen (LCA). CD45 typically consists of an extracellular sequence, proximal to the membrane, which is common to all CD45 isoforms. All the monoclonal antibodies that belong to the CD45 cluster react with this part of the antigen and are able to recognize all CD45 isoforms. These isoforms have extra-cytoplasmic sequences ranging from 391 to 552 amino acids long, with numerous N-linked carbohydrate attachment sites. The cytoplasmic portion contains two phospho-tyrosine-phosphatase domains. Cells expressing CD45 at their surface are all human leucocytes (more precisely, lymphocytes, eosinophils, monocytes, basophils and neutrophils, with different level of expression). This cluster of differentiation is however absent from erythrocytes and platelets. The amino acid sequences of human CD45 are well known in the art, and are for instance available under NCBI accession number NP_001254727.1, or NP_002829.1, or NP_563578.1, or NP_563578.2, or NP_002829.3.

As used herein, “CD3” or “cluster of differentiation 3” is a protein complex and T cell co-receptor that is involved in activating both the cytotoxic T cell (CD8+ naive T cells) and T helper cells (CD4+ naive T cells). It is composed of four distinct chains. In mammals, the complex contains a CD3γ chain, a CD3δ chain, and two CD3ε chains. These chains associate with the T-cell receptor (TCR) and the ζ-chain (zeta-chain) to generate an activation signal in T lymphocytes. The TCR, ζ-chain, and CD3 molecules together constitute the TCR complex. The amino acid sequences of human CD3γ chain, CD3δ chain, CD3 chain are well known in the art, and are for example respectively available under NCBI accession numbers NP_000064.1 (CD3γ), NP_000723.1 (or NP_001035741.1; CD3δ), 1XIW_E (or 1XIW_A; CD3ε).

The terms “CD73” or “cluster of differentiation 73” herein mean a surface enzyme expressed at the surface of several cell types. CD73 is also known as “5′-nucleotidase” (5′-NT), or “ecto-5′-nucleotidase”, or “NT5E”. CD73 contains binding sites for transcription factors AP-2, SMAD proteins, SP-1 and elements responsive to c-AMP, which can be found in c-AMP promoter parts. CD73 catalyses the conversion at neutral pH of purine 5-prime mononucleotides to nucleosides, the preferred substrate being AMP (converted to adenosine by CD73). The CD73 enzyme consists of a dimer of 2 identical 70-kD subunits bound by a glycosyl phosphatidyl inositol linkage to the external face of the plasma membrane. The CD73 enzyme is used as a marker of lymphocyte differentiation. The amino acid sequence of human CD73 is well known in the art, and is for instance available under NCBI accession number NP_001191742.1, or NP_002517.1

As used herein, “CD90”, or “cluster of differentiation 90”, or “THYmocyte differentiation antigen 1”, or Thy-1″, is a 25-37 kDa N-glycosylated, glycophosphatidylinositol (GPI) anchored cell surface protein with a single V-like immunoglobulin domain. CD90 can be used as a marker for a variety of stem cells. The amino acid sequence of human CD 90 is well known in the art, and may be retrieved under NCBI accession number NP_001298089.1, or NP_001298091.1, or NP_006279.1, or NP_001358979.1.

The term “CD68” or “cluster of differentiation 90” herein refers to a type I transmembrane glycoprotein, heavily glycosylated in its extracellular domain. CD68 is also known as GP110, Macrosialin, Scavenger Receptor Class D, Member 1, SCARD1, or LAMP4. CD68 is highly expressed by cells in the monocyte lineage (e.g., monocytic phagocytes, osteoclasts), by circulating macrophages, and by tissue macrophages (e.g., Kupffer cells, microglia). Human CD68 has a molecular weight of 110 kD. Its primary sequence consists of 354 amino acids with predicted molecular weight of 37.4 kD if it were not glycosylated. The amino acid sequence of human CD68 is well known in the art, and may be retrieved under NCBI accession number NP_001035148.1 or NP_001242.1.

As used herein, “CD206”, or “cluster of differentiation 206”, or “mannose receptor” is a type I transmembrane protein, with an extracellular N-terminus and an intracellular C-terminus. CD206 is a C-type lectin primarily present on the surface of macrophages, immature dendritic cells and liver sinusoidal endothelial cells, but is also expressed on the surface of skin cells such as human dermal fibroblasts and keratinocytes.

It is first synthesised as an inactive precursor, but is proteolytically cleaved to its active form in the Golgi apparatus. In general, the extracellular portion of the receptor is composed of 8 consecutive C-type carbohydrate recognition domains (CRDs) closest to the plasma membrane, followed by a single fibronectin type II repeat domain and an N-terminal cysteine-rich domain. CD206 recognises terminal mannose, N-acetylglucosamine and fucose residues on glycans attached to proteins found on the surface of some microorganisms, playing a role in both the innate and adaptive immune systems. Additional functions include clearance of glycoproteins from circulation, including sulphated glycoprotein hormones and glycoproteins released in response to pathological events. The amino acid sequence of human CD206 is well known in the art, and may be retrieved under NCBI accession number NM_002438.1 or NM_006039.1.

The terms “CD24” or “cluster of differentiation CD24” herein mean a sialoglycoprotein anchored via a glycosyl phosphatidylinositol (GPI) link to the cell surface. CD24 is also known as “Signal transducer CD24” or “heat stable antigen CD24 (HSA)”. CD24 is a cell adhesion molecule. CD24 also contributes to a wide range of downstream signalling networks and is crucial for neural development. CD24 is expressed at the surface of most B lymphocytes and differentiating neuroblasts. It is also expressed on neutrophils and neutrophil precursors from the myelocyte stage onwards. The amino acid sequence of human CD24 is well known in the art, and can be retrieved under the NCBI accession number NP_037362.1, or NP_001278666.1, or NP_001278667.1, or NP_001278668.1, or NP_001346013.1.

As used herein, “EpCAM” or “Epithelial cell adhesion molecule” or “CD326” or “cluster of differentiation CD326”, is a glycosylated, 30- to 40-kDa type I membrane protein. This transmembrane glycoprotein is composed of an extracellular domain (242 amino acids) with epidermal growth factor (EGF)—and thyroglobulin repeat-like domains, a single transmembrane domain (23 amino acids), and a short intracellular domain (26 amino acids). EpCAM is also known as TACSTD1 (tumour-associated calcium signal transducer 1), or 17-1A antigen, among others. EpCAM mediates Ca2+-independent homotypic cell-cell adhesion in epithelia. EpCAM is also involved in cell signalling, migration, proliferation, and differentiation. The amino acid sequence of human EpCAM is well known in the art, and can be retrieved under the NCBI accession number NP_002345.1.

A “SSEA-3⁺ cell” is a cell that expresses (and/or exposes/displays) SSEA-3 at the cell surface (e.g., a cell wherein SSEA-3 can be detected at the cell surface using any suitable analytical technology, including any technology mentioned above in relation with the definition of the terms “Cluster of differentiation”, such as FACS, immunofluorescence, immunohistochemistry, etc.). Similarly, a CD105⁺ cell, a CD44⁺ cell, a CD45⁺ cell, a CD73⁺ cell, a CD90⁺ cell, a CD3⁺ cell, a CD68⁺ cell, a CD206⁺ cell, a EpCAM+ cell, or a CD24⁺ cell, is a cell that expresses at the cell surface CD105, CD44, CD45, CD73 CD90, CD3, CD68, CD206, EpCAM, or CD24, respectively (e.g., a cell wherein CD105, CD44, CD45, CD73, CD90, CD3, CD68, CD206, EpCAM, or CD24, respectively, can be detected at the cell surface using any suitable analytical technology mentioned above).

As used herein, a cell “expresses SSEA-3” and/or “exposes SSEA-3” and/or “displays SSEA-3” (or CD105, or CD44, or CD45, or CD73, or CD90, or CD3, or CD68, or CD206, or EpCAM, or CD24, etc.) if SSEA-3 (or respectively, CD105, or CD44, or CD45, or CD73, or CD90, or CD3, or CD68, or CD206, or EpCAM, or CD24, etc.) is present at a significant level on its surface. In particular, a cell expresses SSEA-3 (or respectively, CD105, or CD44, or CD45, or CD73, or CD90, or CD3, or CD68, or CD206, or EpCAM, or CD24, etc.) if the signal associated to surface SSEA-3 (or respectively, CD105, or CD44, or CD45, or CD73, or CD90, or CD3, or CD68, or CD206, or EpCAM, or CD24, etc.) staining (e.g., obtained with an antibody anti-SSEA-3 coupled to a detectable marker (such as a fluorescent marker)) which is measured for said cell is superior to the signal corresponding to the same staining of a cell being known as not expressing SSEA-3 (or respectively, CD105, or CD44, or CD45, or CD73, or CD90, or CD3, or CD68, or CD206, or EpCAM, or CD24, etc.).

Preferably, SSEA-3⁺ cells (or, respectively, CD105⁺ cells, or CD44⁺ cells, CD45⁺ cells, CD73⁺ cells, CD90⁺ cells, CD3⁺ cells, CD68⁺ cells, CD206⁺ cells, EpCAM+ cells, or CD24⁺ cells) are such that the ratio between the surface SSEA-3 (or respectively, CD105, or CD44, or CD45, or CD73, or CD90, or CD3, or CD68, or CD206, or EpCAM, or CD24, etc.)-associated signal measured for said cells and the surface SSEA-3 (or respectively, CD105, or CD44, or CD45, or CD73, or CD90, or CD3, or CD68, or CD206, or EpCAM, or CD24, etc.)-associated signal measured for cells being known as expressing SSEA-3 (or respectively, CD105, or CD44, or CD45, or CD73, or CD90, or CD3, or CD68, or CD206, or EpCAM, or CD24, etc.) is superior or equal to 1 (preferably superior to 10, more preferably superior to 100).

In particular, as used herein, a cell “expresses SSEA-3” and/or “exposes/displays SSEA-3” if SSEA-3 is present at a significant level on its surface (such a cell being also defined as a “SSEA-3⁺ cell”). In particular, a cell expresses SSEA-3 if the signal associated to surface SSEA-3 staining (e.g., obtained with an antibody against SSEA-3 coupled to a detectable marker (such as a fluorescent marker)) which is measured for said cell is higher than the signal corresponding to the same staining of a cell being known as not expressing SSEA-3. Cells that do not express SSEA-3 at their surface are well known in the art. They include for example mesenchymal Stem cells (MSC) and hematopoietic stem cells. In other terms, the ratio between the surface SSEA-3-associated signal measured for said cell and the surface SSEA-3-associated signal measured for at least one cell being known as not expressing SSEA-3 (e.g., MSC and hematopoietic stem cells) is superior to 1 (preferably superior to 10, more preferably superior to 100).

Comparably, as used herein, a cell “expresses CD105” if CD105 is present at a significant level on its surface (such a cell being also defined as a “CD105⁺ cell”). In particular, a cell expresses CD105 if the signal associated to surface CD105staining (e.g., obtained with an antibody against CD105 coupled to a detectable marker (such as a fluorescent marker)) which is measured for said cell is higher than the signal corresponding to the same staining of at least one cell being known as not expressing CD105. Cells that do not express CD105 at their surface are well known in the art. They include for example induced pluripotent stem cells (iPSC). In other terms, the ratio between the surface CD105-associated signal measured for said cell and the surface CD105-associated signal measured for at least one cell being known as not expressing CD105 (e.g., iPSC) is superior to 1 (preferably superior to 10, more preferably superior to 100).

In contrast, a “CD45; cell” or a “CD45neg cell” is a cell that does not expresses CD45 at the cell surface (e.g., a cell wherein CD45 cannot be detected at the cell surface using any suitable analytical technology, including any technology mentioned above in relation with the definition of the terms “Cluster of differentiation”). A cell is said to be “CD45-” if the signal associated to surface CD45 staining (e.g., obtained with an antibody anti-CD45 coupled to a detectable marker (such as a fluorescent marker)) which is measured for said cell is similar or identical to the signal corresponding to the same staining of at least one cell being known as expressing low levels of CD45, or if the signal associated to surface CD45 staining is clearly distinct and lower than the CD45-associated signal measured for at least one cell being known as expressing high level of CD45. Preferably, CD45 cells are such that the ratio between the surface CD45-associated signal measured for these cells and the surface CD45-associated signal measured for a cell being known as highly expressing CD45 is below 1/10. Cells that express high levels of CD45 at their surface are well known in the art. They include for example nucleated hematopoietic cells including myeloid cells and lymphoid cells.

More generally, a cell expressing a protein (or a polypeptide or a peptide), is a cell wherein said protein (or said polypeptide or said peptide) can be detected at the cell surface, or in the cell, or secreted by the cell, or any combination thereof, using any suitable analytical technology, including any technology mentioned above in relation with the definition of the terms “Cluster of differentiation”, such as FACS, immunofluorescence, immunohistochemistry, etc.). Preferably, a cell expresses a protein (or a polypeptide or a peptide), if said protein (or said polypeptide or said peptide) is present at a significant level on its surface, or in the cell, or is secreted at a significant level by the cell, or any combination thereof. In particular, a cell expresses a protein (or a polypeptide or a peptide), if the signal associated to said protein (or said polypeptide or said peptide) which is measured for said cell using any suitable analytical technology is superior to the signal measured using the same analytical technology for a cell being known as not expressing said protein (or said polypeptide or said peptide), preferably the signal is 1.5 times superior, more preferably 2 times superior, more preferably 10 times superior, more preferably 100 times superior.

The term “Ki67” herein refer to a nuclear protein that is associated with cellular proliferation. Ki67 is also associated with ribosomal RNA transcription. Ki67 is also known as Ki-67, or MKI67 (Marker of Proliferation Ki-67). The amino acid sequence of human Ki67 is well known in the art, and can be retrieved under the NCBI accession number NP_001139438.1, or NP_002408.1. A Ki67⁺ cell is a cell expressing a significant level of Ki67 (e.g., a cell wherein Ki67 can be detected using any suitable analytical technology, including any technology mentioned above in relation with the definition of the terms “Cluster of differentiation”, such as FACS, immunofluorescence, immunohistochemistry, etc.). In particular, a cell expresses Ki67 if the signal associated to Ki67 staining (e.g., obtained with an antibody anti-Ki67 coupled to a detectable marker (such as a fluorescent marker)) which is measured for said cell is superior to the signal corresponding to the staining of one cell being known as not expressing Ki67.

Preferably, Ki67⁺ cells are such that the ratio between the Ki67-associated signal measured for said cells and the Ki67-associated signal measured for cells being known as expressing Ki67 is superior or equal to 1 (preferably superior to 10, more preferably superior to 100).

The term “Ly6C” or “lymphocyte antigen 6C” or “urokinase-type plasminogen activator receptor (uPAR)” herein means a protein belonging to the Ly6 family of proteins. Ly6 are cysteine-rich proteins that form disulphide bridges and contain a LU domain (Ly-6 antigen/uPAR domain). The LU domain typically contains 60-80 amino acid residues and contains 10 cysteines arranged in a specific pattern that allows the creation of 5 disulphide bridges which in turn allow the formation of a three-fingered (3F) structural motif. Ly6 proteins are GPI-anchored to the cell membrane or are secreted. Ly6 proteins are expressed in various types of tissues and their expression dependent on the stage of cell differentiation. For example, they are involved in cell proliferation, cell migration, cell-cell interactions, immune cell maturation, macrophage activation, and cytokine production.

A Ly6C^{hi} cell is a cell expressing a significant level of Ly6C (e.g., a cell wherein Ly6C can be detected using any suitable analytical technology, including any technology mentioned above in relation with the definition of the terms “Cluster of differentiation”, such as FACS, immunofluorescence, immunohistochemistry, etc.). In particular, a cell expresses Ly6C if the signal associated to Ly6C staining (e.g., obtained with an antibody anti-Ly6C coupled to a detectable marker (such as a fluorescent marker)) which is measured for said cell is superior to the signal corresponding to the staining of one cell being known as not expressing Ly6C.

Preferably, Ly6C^{hi} cells are such that the ratio between the Ly6C-associated signal measured for said cells and the Ly6C-associated signal measured for cells being known as expressing Ly6C is superior or equal to 1 (preferably superior to 10, more preferably superior to 100).

As used herein, “SRY-box transcription factor 2” or “Sox2” or “Sex determining region Y-box 2” refers to a transcription factor that is essential for maintaining self-renewal, or pluripotency, of undifferentiated embryonic stem cells. Sox2 is a member of the Sox family of transcription factors, which share highly conserved DNA binding domains known as HMG (High-mobility group) box domains containing approximately 80 amino acids. The amino acid sequence of human Sox2 is well known in the art, and can be retrieved under the NCBI accession number NP_003097.1.

The terms “Nanog” or “Homeobox Nanog” or “Homeobox protein NANOG” herein mean a transcriptional factor that helps embryonic stem cells (ESCs) maintain pluripotency by suppressing cell determination factors. The human Nanog protein (hNanog) contains 305 amino acids and possesses 3 functional domains: the N-terminal domain, the C-terminal domain, and the conserved homeodomain motif. The homeodomain region facilitates DNA binding. The N-terminal region of hNanog is rich in serine, threonine and proline residues, and the C-terminus contains a tryptophan-rich domain. The homeodomain in hNanog ranges from residues 95 to 155. The amino acid sequence of human Nanog is well known in the art, and can be retrieved under the NCBI accession number NP_001284627.1, or NP_079141.1.

As used herein, “Octamer-binding transcription factor3/4” or “Oct3/4” or “Oct3 (octamer-binding transcription factor 3)” or “Oct4 (octamer-binding transcription factor 4)” or “POU5F1” (these terms are synonyms) is a homeodomain transcription factor of the POU family, involved in the self-renewal of undifferentiated embryonic stem cells. Oct3/4 comprises an octamer motif, a particular DNA sequence of AGTCAAAT that binds to their target genes and activates or deactivates their expressions. The amino acid sequence of human Oct3/4 is well known in the art, and can be retrieved under the NCBI accession number NP_001167002.1, or NP_001272915.1, or NP_001272916.1, or NP_002692.1, or NP_976034.1.

As used herein “human leukocyte antigen-G5” or “HLA-G5” or “soluble human leukocyte antigen G5” is the isoform 5 of HLA-G histocompatibility antigen. “Human leukocyte antigen-G1” or “HLA-G1” is the isoform 1 of HLA-G histocompatibility antigen. HLA-G belongs to the HLA nonclassical class I heavy chain paralogues. This class I molecule is a heterodimer comprising a heavy chain and a light chain (beta-2 microglobulin). The heavy chain is approximately 45 kDa and is anchored in the membrane. HLA-G is a major immune checkpoint, meaning it downregulates the immune system's response. HLA-G can be expressed under at least seven isoforms through alternative splicing, called HLA-G1, HLA-G2, . . . , HLA-G7. HLA-G5 is soluble. The amino acid sequence of human HLA-G is well known in the art, and can be retrieved under the NCBI accession number NP_002118.1, or NP_001350496.1.

“Intercellular Adhesion Molecule 1” or “ICAM-1” as used herein is a cell surface glycoprotein which is typically expressed on endothelial cells and cells of the immune system. It binds to integrins of type CD11a/CD18, or CD11b/CD18. ICAM-1 is also known as CD54 (Cluster of Differentiation 54). ICAM-1 is a transmembrane protein possessing an amino-terminus extracellular domain, a single transmembrane domain, and a carboxy-terminus cytoplasmic domain. The structure of ICAM-1 is characterized by heavy glycosylation, and the protein's extracellular domain is composed of multiple loops created by disulphide bridges within the protein. The dominant secondary structure of the protein is the beta sheet. ICAM-1 is an intercellular adhesion molecule continuously present in low concentrations in the membranes of leukocytes and endothelial cells. The amino acid sequence of human ICAM-1 is well known in the art, and can be retrieved under the NCBI accession number NP_000192.1.

As used herein, “Arginase 1” or “Arg1” is a manganese-containing enzyme, belonging to the ureohydrolase family of enzymes. Arginase catalyses the final step in the urea cycle, a series of biochemical reactions in mammals during which the body disposes of harmful ammonia. Specifically, Arginase 1 catalyses the hydrolysis of arginine to ornithine and urea. The amino acid sequence of human Arg1 is well known in the art, and can be retrieved under the NCBI accession number NP_000036.1, or NP_001231367.1, or NP_001355949.1.

“Nitric Oxide Synthase 2” or “Nos2” as used herein refers to an enzyme catalysing the synthesis of nitric oxide (a reactive free radical involved in neurotransmission, antimicrobial and antitumoral activities). Nos2 is expressed in epithelial cells of the liver, lung and bone marrow. It is inducible by a combination of lipopolysaccharide and cytokines. The amino acid sequence of human Nos2 is well known in the art, and can be retrieved under the NCBI accession number NP_000616.1.

“Monocyte-Chemoattractant protein-1” or “MCP1” as used herein is a cytokine that belongs to the CC chemokine family. MCP1 is also referred to as chemokine (C-C motif) ligand 2 (CCL2) and small inducible cytokine A2. MCP1 is a monomeric polypeptide, with a molecular weight of approximately 13-15 kDa depending on levels of glycosylation. MCP1 is anchored in the plasma membrane of endothelial cells by glycosaminoglycan side chains of proteoglycans. It is primarily secreted by monocytes, macrophages and dendritic cells. MCP1 gene can be induced by Platelet derived growth factor. CCR2 and CCR4 are two cell surface receptors that bind MCP1. MCP1 recruits monocytes, memory T cells, and dendritic cells to the sites of inflammation produced by either tissue injury or infection.

The amino acid sequence of human MCP1 is well known in the art, and can be retrieved under the NCBI accession number NP_002973.1.

As used herein, “Zonula occludens-1” or “ZO-1” is a 220-kD peripheral membrane protein ZO-1 is also known as Tight junction protein-1 (TJP1). ZO-1 is generally located on a cytoplasmic membrane surface of intercellular tight junctions. It has a role as a scaffold protein which cross-links and anchors Tight Junction (TJ) strand proteins, which are fibril-like structures within the lipid bilayer, to the actin cytoskeleton. It may also be involved in signal transduction at cell-cell junctions. The amino acid sequence of human ZO-1 is well known in the art, and can be retrieved under the NCBI accession number NP_001287954.1, or NP_001287955.1, or NP_001317168.1, or NP_003248.1, or NP_783297.1.

“Interleukin 6” or “IL-6” or “IL6” is an interleukin that acts as both a pro-inflammatory cytokine and an anti-inflammatory myokine. IL-6 is secreted by macrophages in response to specific microbial molecules, referred to as pathogen-associated molecular patterns (PAMPs). IL-6's role as an anti-inflammatory myokine is mediated through its inhibitory effects on TNF-alpha and IL-1 and its activation of IL-1ra and IL-10.IL-6 is responsible for stimulating acute phase protein synthesis, as well as the production of neutrophils in the bone marrow. It supports the growth of B cells and is antagonistic to regulatory T cells. The amino acid sequence of human IL6 is well known in the art, and can be retrieved under the NCBI accession number NP_000591.1, or NP_001305024.1, or NP_001358025.1.

As used herein, “PD-L1” or “Programmed death-ligand 1” is a 40 kDa type 1 transmembrane protein. PD-L1 is also known as cluster of differentiation 274 (CD274) or B7 homolog 1 (B7-H1). PD-L1 has been speculated to play a major role in suppressing the adaptive arm of immune systems during particular events such as pregnancy, tissue allografts, autoimmune disease and other disease states such as hepatitis. Normally the adaptive immune system reacts to antigens that are associated with immune system activation by exogenous or endogenous danger signals. In turn, clonal expansion of antigen-specific CD8+ T cells and/or CD4+ helper cells is propagated. The binding of PD-L1 to the inhibitory checkpoint molecule PD-1 transmits an inhibitory signal based on interaction with phosphatases (SHP-1 or SHP-2) via Immunoreceptor Tyrosine-Based Switch Motif (ITSM). This reduces the proliferation of antigen-specific T-cells in lymph nodes, while simultaneously reducing apoptosis in regulatory T cells (anti-inflammatory, suppressive T cells). The amino acid sequence of human PD-L1 is well known in the art, and can be retrieved under the NCBI accession number NP_001254635.1, or NP_001300958.1, or NP_054862.1.

The term “monocytes” herein refers to a type of leukocytes (representing 2 to 10% of circulating leukocytes, 0.1 to 1×10⁹/L in human peripheral blood) produced by the bone marrow from hematopoietic stem cells. They circulate in the blood, typically between one and 7 days, and most of them migrate into tissues where they differentiate, generating so-called “monocyte-derived cells” with a macrophage phenotype. Monocytes belong to the family of the “peripheral mononuclear cell of the blood (PBMCs)” (or “peripheral mononuclear blood cells”). PBMCs are a critical component in the immune system to fight infection and adapt to intruders. These cells can be extracted from whole blood using ficoll, a hydrophilic polysaccharide that separates layers of blood, which will separate the blood into a top layer of plasma, followed by a layer of PBMCs and a bottom fraction of polymorphonuclear cells (such as neutrophils and eosinophils) and erythrocytes.

Monocytes are variable in size and appearance, but they show common expression of a number of markers, including cell surface antigens (or receptors). The markers expressed by monocytes are known in the art (e.g., Zawada et al., Blood 118 (12): e50-61, 2011; Ziegler-Heitbrock et al., Blood, 116 (16): e74-80, 2010; Wong et al., Blood, 118 (5): e16-31, 2011). In particular, monocytes are easily identified by specific antigens (including cell surface antigens, e.g., CD14, CD16, HLA-DR, any combination thereof) combined with morphometric characteristics (e.g., size, shape, granulometry, etc.), using any suitable analytical technology, including any technology mentioned above in relation with the definition of the terms “Cluster of differentiation” (such as FACS, immunofluorescence, immunohistochemistry, etc.). Monocytes can also express the HLA-DR (Human Leukocyte Antigen-DR isotype) cell surface receptor. These monocytes are referred to as HLA-DR+ monocytes. “HLA-DR” or “Human leukocyte antigen-DR isotype” is an MHC (major histocompatibility complex) class II cell surface receptor encoded by the human leukocyte antigen complex on chromosome 6 region 6p21.31. The complex of HLA-DR and peptide, generally between 9 and 30 amino acids in length, constitutes a ligand for the T-cell receptor (TCR). The primary function of HLA-DR is to present peptide antigens, potentially foreign in origin, to the immune system for the purpose of eliciting or suppressing T-(helper)-cell responses that eventually lead to the production of antibodies against the same peptide antigen. Antigen presenting cells (macrophages, B-cells and dendritic cells) are the cells in which DR are typically found. HLA-DR is an αβ heterodimer, cell surface receptor, each subunit of which contains two extracellular domains, a membrane-spanning domain and a cytoplasmic tail. The reference amino acid sequence for human HLA-DR alpha and beta chains can be represented by the NCBI accessions AAA36275.1 or AAA59785.1 or AAA36302.1 (alpha chain) and AAA58651.1 or AAA59816.1 (beta chain). “Ly6C^{hi} monocytes” are monocytes expressing a significant level of Ly6C.

“T cells” or “T lymphocytes” are a type of lymphocyte that plays a central role in cell-mediated immunity. They can be distinguished from other lymphocytes, such as B cells and natural killer cells (NK cells), by the presence of a T-cell receptor (TCR) on the cell surface. Other antigens expressed by T lymphocytes include, but are not limited to CD2, CD3, etc. “CD3⁺ T cells” are T cells expressing a significant level of CD3.

As used herein, “macrophages” are a type of white blood cell of the innate immune system that engulfs and digests pathogens, such as cancer cells, microbes, cellular debris, and foreign substances, which do not have proteins that are specific to healthy body cells on their surface. The process is called phagocytosis, which acts to defend the host against infection and injury. Macrophages are found in essentially all tissues, where they patrol for potential pathogens by amoeboid movement. They take various forms (with various names) throughout the body (e.g., histiocytes, Kupffer cells, alveolar macrophages, microglia, and others). Besides phagocytosis, they play a critical role in nonspecific defence (innate immunity) and also help initiate specific defence mechanisms (adaptive immunity) by recruiting other immune cells such as lymphocytes. For example, they are important as antigen presenters to T cells. Beyond increasing inflammation and stimulating the immune system, macrophages also play an important anti-inflammatory role and can decrease immune reactions through the release of cytokines. Macrophages that encourage inflammation are called M1 macrophages, whereas those that decrease inflammation and encourage tissue repair are called M2 macrophages. This difference is reflected in their metabolism; M1 macrophages have the unique ability to metabolize arginine to the “killer” molecule nitric oxide, whereas M2 macrophages have the unique ability to metabolize arginine to the “repair” molecule ornithine.

Human macrophages are about 21 micrometres in diameter and are produced by the differentiation of monocytes in tissues. They can be identified using any suitable analytical technology, including any technology mentioned above in relation with the definition of the terms “Cluster of differentiation” (such as FACS, immunofluorescence, immunohistochemistry, etc., in particular flow cytometry or immunohistochemical staining) by their specific expression of proteins such as CD14, CD40, CD11b, CD64, F4/80 (mice)/EMR1 (human), lysozyme M, MAC-1/MAC-3, CD68, and any combination thereof.

By “CD68⁺CD206⁺ (M2-like) macrophages” or “CD68⁺CD206⁺macrophages” it is herein referred to as macrophages expressing CD68 and CD206. CD68⁺CD206⁺macrophages may be considered as M2 macrophages.

By “Ki67⁺CD24⁺ cells” it is herein referred to as cells expressing Ki67 and CD24.

The “gastrointestinal tract” is composed of numerous cell types that are important for immune activation and barrier surface defences. The “gastrointestinal epithelium” is composed of enterocytes, goblet cells, Paneth cells, enteroendocrine cells, tuft cells, and stem cells. In contrast, the lamina propria is composed of immune cells such as dendric cells, T cells, and macrophages.

“Paneth cells” herein means cells in the small intestine epithelium, below the intestinal stem cells in the intestinal glands (also called crypts of Lieberkühn), alongside goblet cells, enterocytes, and enteroendocrine cells. Paneth cells are found throughout the small intestine and some Paneth cells can also be found in the cecum and appendix at the base of the intestinal glands. The Paneth cell increase in numbers towards the end of the small intestine. Like the other epithelial cell lineages in the small intestine, Paneth cells originate at the stem cell region near the bottom of the gland. There are on average 5-12 Paneth cells in each small intestinal crypt. Unlike the other epithelial cell types, Paneth cells migrate downward from the stem cell region and settle just adjacent to it.

Paneth cells comprise large eosinophilic refractile granules that occupy most of their cytoplasm. These granules contain several anti-microbial compounds and other compounds that are known to be important in immunity and host-defence. When exposed to bacteria or bacterial antigens, Paneth cells secrete some of these compounds into the lumen of the intestinal gland, thereby contributing to maintenance of the gastrointestinal barrier by controlling the enteric bacteria. Therefore, Paneth cells play a role in the innate immune system. Paneth cells are stimulated to secrete defensins when exposed to bacteria or bacterial products as lipopolysaccharide, lipoteichoic acid, muramyl dipeptide and lipid A. They are also stimulated by cholinergic signalling normally preceding the arrival of food and potentially a new bacterial load. Paneth cells sense bacteria via MyD88-dependent toll-like receptor (TLR) activation which then triggers antimicrobial action. The principal defence molecules secreted by Paneth cells are alpha-defensins, which are known as cryptdins in mice. These peptides have hydrophobic and positively charged domains that can interact with phospholipids in cell membranes. This structure allows defensins to insert into membranes, where they interact with one another to form pores that disrupt membrane function, leading to cell lysis. Due to the higher concentration of negatively charged phospholipids in bacterial than vertebrate cell membranes, defensins preferentially bind to and disrupt bacterial cells, sparing the cells they are functioning to protect.

“Small intestinal crypts” house stem cells (called “intestinal crypt stem cells”) that serve to constantly replenish epithelial cells that die and are lost from the villi. Paneth cells support the physical barrier of the epithelium by providing essential niche signals to their neighbouring intestinal stem cells. Protection and stimulation of these stem cells is essential for long-term maintenance of the intestinal epithelium, in which Paneth cells play a critical role.

By “intestinal crypt” or “intestinal gland” (also known as crypt of Lieberkühn), it is herein referred to a gland found in between villi in the intestinal epithelium lining of the small intestine and large intestine (or colon). The glands and intestinal villi are covered by epithelium, which contains multiple types of cells: enterocytes (absorbing water and electrolytes), goblet cells (secreting mucus), enteroendocrine cells (secreting hormones), cup cells, tuft cells, and at the base of the gland, Paneth cells (secreting anti-microbial peptides) and stem cells. New epithelium is formed in the intestinal crypts. The basal (further from the intestinal lumen) portion of the crypt contains multipotent stem cells. During each mitosis, one of the two daughter cells remains in the crypt as a stem cell, while the other differentiates and migrates up the side of the crypt and eventually into the villus. These stem cells can differentiate into either an absorptive (enterocytes) or secretory (Goblet cells, Paneth cells, enteroendocrine cells) lineages.

By “intestinal villi” or “villi” it is herein referred to as small, finger-like projections that extend into the lumen of the small intestine. Each villus is approximately 0.5-1.6 mm in length (in humans), and has many microvilli projecting from the enterocytes of its epithelium which collectively form the striated or brush border. Each of these microvilli are about 1 μm in length, around 1000 times shorter than a single villus. Villi increase the internal surface area of the intestinal walls making available a greater surface area for absorption (notably absorption of nutrients). The villi are connected to the blood vessels, so that the circulating blood can carry the nutrients away.

The terms “clonogenic crypt” or “clonogenic crypt cells” herein mean intestinal crypts comprising regenerative stem cells. Regenerative stem cells play a role in intestinal regeneration process. As intestinal regeneration process often gives rise to clones or colonies of cells (depending on the situation, in vivo, in vitro, etc.), regenerative stem cells are often being referred to as clonogenic cells.

The terms “clonogenic crypt count” herein mean the number (or amount or quantity) of clonogenic crypts. Methods for identifying and counting clonogenic crypts are known in the art. Clonogenic crypts may be for instance identified and/or counted on histological sections of the intestine, in particular of the ileum. The main criteria generally used to count a clonogenic crypt is 8 or more contiguous epithelial cells and at least 2 Paneth cells recognizable by their intracytoplasmic granules.

The histological section may be coloured, notably to distinguish the different cell types. Examples of colorants that may be used include haematoxylin, eosin, saffron, and any combination thereof.

An example of method that can be used to identify and count clonogenic crypt comprises the following steps. A) A section of the ileum (e.g., a 0.5 to 10 cm section, preferably about 1 cm section) is cut and placed in a fixative (e.g., paraformaldehyde (PFA), at a concentration of 1 to 10%, preferably 4%). B) After fixation, the sections are embedded in paraffin to generate cross-sections during cutting and then stained with hematoxylin/esonin/safran. The number of clonogenic crypts are counted on the entire section, based on the above-mentioned criteria. As used herein, “Wharton's jelly” is a gelatinous substance within the umbilical cord, largely made up of mucopolysaccharides (hyaluronic acid and chondroitin sulphate). It acts as a mucous connective tissue containing some fibroblasts and macrophages, and is derived from extra-embryonic mesoderm of the connecting stalk. Wharton's jelly is also called substantia gelatinea funiculi umbilicali. As a mucous connective tissue, it is rich in proteoglycans, and protects and insulates umbilical blood vessels. Wharton's jelly contains adult stem cells.

Muse Cells for Use for Treating a Pathology Selected from Radiation-Induced Pathologies and Pathologies of the Intestine-Methods of Treatment

In the context of the present invention, the Inventors have designed a novel treatment of radiation-induced pathologies and pathologies of the intestine. More specifically, the Inventors surprisingly found that administering a population of cells, called Multilineage-differentiating stress-enduring (Muse) cells, significantly increases survival of irradiated mice. The Muse cells promote rapid and durable regeneration of injured tissue, especially injured intestine tissue, as supported by the experimental data. In particular, the Inventors demonstrated for the first time that a single injection of a small quantity of Muse cells (only 50,000 Muse cells) generates a beneficial stem cell microenvironment, favouring the reconstitution of intestinal epithelial barrier, leading ultimately to full recovery and survival following irradiation (IR).

The data obtained by the Inventors unexpectedly highlight that Muse cells migrate into irradiated tissue, such as irradiated small intestine and persist into the crypts. The results show that Muse cells display immunosuppressive properties through cell contact and/or soluble mediators, which are expressed either constitutively or induced by inflammation. These immunosuppressive properties regulate their integration in injured tissue after injection and lead to inhibition of inflammation responses and to stimulation of the regenerative process. In small intestine of Muse-treated mice, an early enhanced secretion of IL-6 and MCP-1 cytokines was observed associated with (i) recruitment of monocytes/M2-like macrophages and (ii) proliferation of Paneth cells through activation of the IL-6/Stat3 pathway.

The data further show that Muse cells promote the regeneration of intestinal epithelium characterized by (i) a hyperproliferation of crypt cells, (ii) an increased expression of the tight junction protein ZO-1 and (iii) an increased expression of adherent protein EpCAM. Through this breakthrough work, the Inventors thus provide for the first time an original, efficient and easy therapeutic strategy for treating lethal radiation-induced pathologies as well as pathologies of the intestine.

Accordingly, the present invention thus relates to Multilineage-differentiating stress enduring (Muse) cells for use for treating a pathology selected from radiation-induced pathologies and pathologies of the intestine, wherein the Muse cells are SSEA-3⁺CD105⁺. In other words, the Muse cells of the invention are characterized by the surface markers SSEA-3⁺ and CD105⁺ (i.e., the Muse cells express (and/or preferably display at their cell surface) the markers SSEA-3 and CD105).

The present invention also concerns the use of Muse cells for treating a pathology selected from radiation-induced pathologies and pathologies of the intestine (preferably comprising the administration of a therapeutically effective amount of Muse cells to a subject in need thereof).

The present invention also concerns the use of Muse cells for manufacturing a medicament for treating a pathology selected from radiation-induced pathologies and pathologies of the intestine.

The present invention also provides a method for treating a pathology selected from radiation-induced pathologies and pathologies of the intestine, comprising the administration of a therapeutically effective amount of Muse cells to a subject in need thereof.

The Muse cells are preferably mammal Muse cells, more preferably human Muse cells. The present invention thus relates to human Multilineage-differentiating stress enduring (Muse) cells, for use for treating a pathology selected from radiation-induced pathologies and pathologies of the intestine, wherein the Muse cells are SSEA-3⁺CD105⁺.

The radiation-induced pathology is preferably selected from the group consisting of a radiation-induced tissue lesion, and a radiation-induced gastrointestinal syndrome (GIS). The pathology of the intestine is preferably selected from the group consisting of colitis, ulcer of the intestine, inflammatory bowel disease (such as ulcerative colitis (UC), Crohn's disease (CD)), radiation-induced gastrointestinal syndrome (GIS), cancer of the intestine, and lesion of the intestine. In a particularly preferred embodiment, the pathology is a radiation-induced gastrointestinal syndrome (GIS).

Accordingly, in a particularly preferred embodiment, the present invention relates to Muse cells for use for treating radiation-induced gastrointestinal syndrome (GIS), wherein the Muse cells are SSEA-3⁺CD105⁺. The present invention also preferably concerns the use of Muse cells for treating GIS (preferably comprising the administration of a therapeutically effective amount of Muse cells to a subject in need thereof). The present invention also preferably concerns the use of Muse cells for manufacturing a medicament for treating GIS. The present invention also preferably provides a method for treating GIS, comprising the administration of a therapeutically effective amount of Muse cells to a subject in need thereof.

The radiation (i.e. the radiation inducing the radiation-induced pathology, including radiation-induced tissue lesion, and/or GIS) is preferably selected from the group consisting of:
electromagnetic radiation, preferably selected from the group consisting of radio waves, microwaves, infrared, visible light, ultraviolet, x-rays, and gamma radiation (γ); more preferably selected from the group consisting of radio waves, microwaves, infrared, visible light, x-rays, and gamma radiation (γ);
particle radiation, preferably selected from the group consisting of alpha radiation (a), beta radiation (B), proton radiation, and neutron radiation (particles of non-zero rest energy);
acoustic radiation, preferably selected from the group consisting of ultrasound, sound, and seismic waves (dependent on a physical transmission medium);
gravitational radiation, that takes the form of gravitational waves, or ripples in the curvature of spacetime; and
any combination thereof.

In a preferred embodiment, the Muse cells are further characterized by a surface marker selected from: CD44⁺, CD73⁺, CD90⁺, CD45, and any combination thereof. Alternatively, the Muse cells are further characterized by a surface marker selected from: CD44⁺, CD73⁺, CD90⁺, CD45⁺, and any combination thereof.

In a particularly preferred embodiment, the Muse cells of the invention are SSEA-3⁺CD105⁺CD44⁺CD73⁺CD90⁺CD45. In an alternative particularly preferred embodiment, the Muse cells of the invention are SSEA-3⁺CD105⁺CD44⁺CD73⁺CD90⁺CD45⁺.

The Muse cells may be obtained from adult or embryonic/foetal tissue, such as bone marrow, peripheral blood, adipose tissue, umbilical cord, or any mixture thereof. Indeed, the data obtained by the Inventors show that active Muse cells can be obtained from any of these tissues and that irradiation induced-damages are efficiently treated regardless of the source of Muse cells. Indeed, the experimental results show that the properties, the marker and protein expression profiles, the physiology, the pluripotent potential, and the capacities (such as multilineage differentiation capacity) of Muse cells obtained from adult tissue are comparable to those obtained from embryonic/foetal tissue. The data further demonstrate that the proportion of Muse cells is particularly high in umbilical cord, more specifically in Wharton's jelly of umbilical cord. Accordingly, the Muse cells are preferably obtained from umbilical cord, more preferably from Wharton's jelly of umbilical cord.

The Muse cells may be obtained/isolated from any of the above-mentioned tissue, using any of the technologies listed above for detecting CD molecules (and more generally cell surface markers), in the section “Definitions”. Accordingly, the Muse cells may be detected (or selected, or isolated, or obtained, or any combination thereof), by detecting (or selecting, or isolating, or obtaining, or any combination thereof) cells expressing SSEA-3⁺ and CD105⁺, from any of the above-mentioned tissues.

The Muse cells may also be obtained from Mesenchymal Stem cells (MSC). Methods and means for obtaining Muse cells from MSC are well known in the art. Typically, MSC (preferably at a density ranging from 1,000 cells/cm² to 50,000 cells/cm², more preferably from 5,000 cells/cm² to 40,000 cells/cm², more preferably from 10,000 cells/cm² to 30,000 cells/cm², more preferably from 12,000 cells/cm² to 20,000 cells/cm², even more preferably at an approximate density of 15,000 cells/cm²) may be grown at approximately 37° C., in about 95% air and about 5% CO₂, in the following medium (herein called Muse cell culture medium):
Low-glucose Dulbecco's Modified Eagle's Medium (DMEM), i.e., a medium comprising from 100 mg/L to 3000 mg/L glucose (preferably from 300 mg/L to 2500 mg/L glucose, more preferably from 500 mg/L to 2000 mg/L glucose, more preferably from 800 mg/L to 1500 mg/L glucose, more preferably from 900 mg/L to 1200 mg/L glucose, even more preferably about 1000 mg/L glucose);
supplemented or not with L-glutamine, sodium bicarbonate, folic acid, and any combination thereof);
supplemented with a dipeptide of L-alanyl-L-glutamine and sodium chloride (NaCl), such as the commercially available supplement commercialised under the name GlutaMAX™;
supplemented with foetal bovine serum (FBS), preferably from 5 to 15% FBS (preferably from 7 to 12% FBS, more preferably about 10% FBS);
supplemented with Fibroblast growth factor 2 (FGF-2) (preferably human FGF-2) preferably from 0.1 to 10 ng/ml FGF-2 (preferably from 0.5 to 5 ng/ml FGF-2, more preferably about 1 ng/ml FGF-2);
supplemented with kanamycin sulphate (KS), preferably from 0.01 to 1 mg/ml KS (preferably from 0.05 to 0.5 mg/ml KS, more preferably about 0.1 mg/ml KS).

MSC may be obtained from adult or embryonic/foetal tissue, such as bone marrow, peripheral blood, adipose tissue, umbilical cord, or any mixture thereof.

Methods and means for culturing/growing/maintaining Muse cells are well known in the art. Typically, Muse cells (preferably at a density ranging from 1,000 cells/cm² to 50,000 cells/cm², more preferably from 5,000 cells/cm² to 40,000 cells/cm², more preferably from 10,000 cells/cm² to 30,000 cells/cm², more preferably from 12,000 cells/cm² to 20,000 cells/cm², even more preferably at an approximate density of 15,000 cells/cm²) may be grown at approximately 37° C., in about 95% air and about 5% CO₂, in the Muse cell culture medium (as defined above, in the previous paragraph).

The experimental data demonstrate that Muse cells express high levels of proteins characteristic of an immature status, such as Sox2, Nanog and Oct3/4. Thus, the Muse cells advantageously express at least one gene selected from the group consisting of the genes encoding:
SRY-box transcription factor 2 (Sox2),
Nanog homeobox (Nanog),
Octamer-binding transcription factor3/4 (Oct3/4), and
Any combination thereof.

In one embodiment, the Muse cells express the gene encoding soluble human leukocyte antigen-G5 (HLA-G5), as supported by the experimental data obtained by the Inventors. Therefore, the Muse cells advantageously express at least one gene selected from the group consisting of the genes encoding:
SRY-box transcription factor 2 (Sox2),
Nanog homeobox (Nanog),
Octamer-binding transcription factor 3/4 (Oct3/4), and
Soluble human leukocyte antigen-G5 (HLA-G5).

The Inventors demonstrated for the first time that Muse cells display immunosuppressive properties through cell contact and/or soluble mediators, which are expressed either constitutively or induced by inflammation. These immunosuppressive properties regulate their integration in injured tissue after injection and lead to inhibition of inflammation responses and to stimulation of the regenerative process. Indeed, the experimental data further show that Muse cells express Soluble human leukocyte antigen-G1 (HLA-G1), Human Leukocyte Antigen-DR isotype (HLA-DR) and Programmed death-ligand 1 (PD-L1), only when Muse cells are pre-activated (primed) with the IFNγ and TNFα pro-inflammatory cytokines. Accordingly, Muse cells preferably express at least one gene selected from the group consisting of the genes encoding HLA-G1, HLA-DR and PD-L1, when Muse cells are in presence of (or exposed to, or pre-activated with, or primed with, or stimulated with) the IFNγ and/or TNFα pro-inflammatory cytokines. The thusly pre-activated Muse cells preferably express genes encoding immunosuppressive factors (such as indoleamine 2,3-dioxygenase (IDO), cyclooxygenase 2 (Cox2), PD-L1, Transforming growth factor beta 1 (TGFβ1), and any combination thereof) at higher levels than naive cells (i.e., Muse cells that have not been in presence of (or exposed to, or pre-activated with, or primed with, or stimulated with) the IFNγ and/or TNFα pro-inflammatory cytokines). In other word, Muse cells pre-activated with the IFNγ and/or TNFα pro-inflammatory cytokines preferably overexpress genes encoding immunosuppressive factors (such as IDO, Cox2, PD-L1, TGFβ1, and any combination thereof) compared to naive cells (i.e., Muse cells not pre-activated with the IFNγ and/or TNFα pro-inflammatory cytokines).

In a preferred embodiment, Muse cells pre-activated with the IFNγ and/or TNFα pro-inflammatory cytokines, express genes encoding any of:
IDO, at least 50-fold (more preferably at least 70-fold, more preferably at least 80-fold, more preferably at least 84-fold) higher than naïve cells;
Cox2, at least 3-fold (more preferably at least 5-fold, more preferably at least 10-fold, more preferably at least 14.5-fold) higher than naïve cells;
PD-L1, at least 2-fold (more preferably at least 4-fold, more preferably at least 6-fold, more preferably at least 8.8-fold) higher than naïve cells;
TGFβ1, at least 1.2-fold (more preferably at least 1.5-fold, more preferably at least 1.7-fold, more preferably at least.8-fold) higher than naïve cells; and
Any combination thereof.

Methods and means for pre-activating Muse cells with TNFα and/or IFNγ are well known in the art. Typically, the Muse cells may be grown in presence of TNFα (at a concentration ranging from 1 to 100 ng/ml, preferably from 5 to 80 ng/ml, more preferably from 10 to 50 ng/ml, more preferably from 12 to 30 ng/ml, even more preferably a concentration of approximately 15 ng/ml) and/or IFNγ (at a concentration ranging from 0.5 to 100 ng/ml, preferably from 2 to 80 ng/ml, more preferably from 5 to 50 ng/ml, more preferably from 8 to 30 ng/ml, even more preferably a concentration of approximately 10 ng/ml) (typically TNFα and/or IFNγ is/are added in the Muse cell culture medium), for a period ranging from 12 h to 96h, preferably from 24 h to 84h, more preferably from 36 h to 72h, more preferably from 48 h to 60h, even more preferably for approximately 48h), at approximately 37° C., in about 95% air and about 5% CO₂.

The experimental results unexpectedly showed that WJ-Muse cells express proteins that interplay with the innate immune response that can modulate the healing process, and proteins involved in intestinal barrier regeneration. Such proteins are highly useful for treating radiation-induced pathologies and pathologies of the intestine.

Thus, in an advantageous embodiment, the Muse cells express proteins active in cellular pathways selected from the group consisting of angiogenesis, cell adhesion, cell migration, response to drug. Such proteins include notably proteins related to collagen, to extracellular matrix, to development of tissues, to anti-oxidant activity, to adaptive immunity, and any combination thereof. Alternatively or in combination, the Muse cells advantageously express proteins active in cellular pathways selected from the group consisting of innate immune response, and intestinal barrier function (such as the Intercellular Adhesion Molecule 1 (ICAM-1) protein). According to this embodiment, wherein the Muse cells advantageously express proteins active in cellular pathways selected from the group consisting of innate immune response and intestinal barrier function, the Muse cells are preferably obtained from embryonic/foetal tissue, such as bone marrow, peripheral blood, adipose tissue, umbilical cord (more preferably obtained from umbilical cord, even more preferably from Wharton's jelly of umbilical cord).

The Muse cells are advantageously pluripotent. As demonstrated by the experimental data, the Muse cells have preferably the ability to differentiate in any cell type selected from the group consisting of adipocytes, osteoclasts, and epithelial cells. More preferably the Muse cells differentiate in any cell type selected from the group consisting of adipocytes, osteoclasts, and epithelial cells. Even more preferably, the Muse cells differentiate (i.e., the Muse cells have preferably the ability to differentiate) in any cell type selected from the group consisting of adipocytes, osteoclasts, and epithelial cells, in presence of a specific differentiation media. Methods and means for inducing Muse cells differentiation (i.e., for differentiating Muse cells) are well known in the art. Typically, the Muse cells (preferably at a density ranging from 1,000 cells/cm² to 50,000 cells/cm², more preferably from 5,000 cells/cm² to 40,000 cells/cm², more preferably from 10,000 cells/cm² to 30,000 cells/cm², more preferably from 12,000 cells/cm² to 20,000 cells/cm², even more preferably at an approximate density of 15,000 cells/cm²) may be grown at approximately 37° C., in about 95% air and about 5% CO₂ in the following exemplary conditions:
Epithelial differentiation: Muse cells may be grown/cultured in the Muse cell culture medium (as defined above), supplemented with 10 UM retinoic acid, for a period of 1 to 8 week(s), preferably from 2 to 7 weeks, more preferably from 3 to 6 weeks, even more preferably for about 4 weeks; Osteogenic differentiation: Muse cells may be grown/cultured in a Osteogenic/Adipogenic Base Media, supplemented with osteogenic supplements and kanamycin sulphate (KS), preferably from 0.01 to 1 mg/ml KS (preferably from 0.05 to 0.5 mg/ml KS, more preferably about 0.1 mg/ml KS) for a period of 0.5 to 6 week(s), preferably from 1 to 5 weeks, more preferably from 2 to 4 weeks, even more preferably for about 2 weeks;
Adipogenic differentiation: Muse cells may be grown/cultured in a Osteogenic/Adipogenic Base Media, supplemented with adipogenic supplements and kanamycin sulphate (KS), preferably from 0.01 to 1 mg/ml KS (preferably from 0.05 to 0.5 mg/ml KS, more preferably about 0.1 mg/ml KS) for a period of 0.5 to 6 week(s), preferably from 1 to 5 weeks, more preferably from 2 to 4 weeks, even more preferably for about 3 weeks.

The Inventors demonstrated for the first time that Muse cells display immunosuppressive properties through cell contact and/or soluble mediators, which are expressed either constitutively or induced by inflammation. These immunosuppressive properties regulate their integration in injured tissue after injection and lead to inhibition of inflammation responses and to stimulation of the regenerative process. As demonstrated by the data, the Muse cells advantageously have the ability to reduce (or reduce) CD3⁺T-cell proliferation in presence of concanavalin A-stimulated human peripheral blood mononuclear cells (hPBMC) and/or of concanavalin A-stimulated murine spleen lymphocytes (mSL). Accordingly, the Muse cells preferably reduce CD3⁺T-cell proliferation in presence of concanavalin A-stimulated hPBMC and/or of concanavalin A-stimulated mSL, compared to concanavalin A-stimulated hPBMC and/or mSL alone (i.e., CD3⁺T-cell proliferation in presence of concanavalin A-stimulated hPBMC and/or of concanavalin A-stimulated mSL alone, without Muse cells).

The data surprisingly highlight that Muse cells can drive macrophage polarization toward anti-inflammatory M2-like phenotype (in particular through overexpression of Arg1 by macrophages), known to be involved in tissue remodelling and repair. Thus, in a preferred embodiment, the Muse cells are capable of inducing the increase (or are capable of increasing, or induce the increase, or increase) the levels of expression, by macrophages, of the gene encoding Arginase 1 (Arg1) (preferably compared to the levels of expression, by macrophages, of the gene encoding Arg1 in the absence of Muse cells). Advantageously, co-culture of macrophages with Muse cells highly increases the levels of expression, by macrophages, of the gene encoding Arginase 1 (Arg1). Preferably, Muse cells increase the levels of expression, by macrophages, of the gene encoding Arg1 by at least 100-fold (preferably by at least 500-fold, more preferably by at least 700-fold, more preferably by at least 800-fold, even more preferably by about 843-fold).

The Muse cells may also be capable of inducing the increase (or are capable of increasing, or induce the increase, or increase) the levels of expression, by macrophages, of the gene encoding the gene encoding Nitric Oxide Synthase 2 (Nos2) (preferably compared to the levels of expression, by macrophages, of the gene encoding Nos2 in the absence of Muse cells). Advantageously, co-culture of macrophages with Muse cells increases the levels of expression, by macrophages, of the gene encoding Nos2. Preferably, Muse cells increase the levels of expression, by macrophages, of the gene encoding Nos2 by at least 10-fold (preferably by at least 20-fold, more preferably by at least 30-fold, even more preferably by about 35-fold).

It is particularly preferred that the increase of macrophage Arg1 expression levels induced by Muse cells is significantly higher than the increase of macrophage Nos2 expression levels induced by Muse cells. Indeed, it is preferred that the Muse cells polarize (or direct) macrophages toward anti-inflammatory M2-like phenotype.

Thus, the Muse cells advantageously increase the levels of expression, by macrophages, of the gene encoding Arginase 1 (Arg1) and/or the gene encoding Nitric Oxide Synthase 2 (Nos2) (compared to the levels of expression, by macrophages, of the gene encoding Arg1 and/or the gene encoding Nos2, in the absence of Muse cells), preferably wherein the Muse cells increase the levels of expression, by macrophages, of the gene encoding Arg1.

In an advantageous embodiment, the Muse cells are capable of migrating toward and integrating (or the Muse cells migrate toward and integrate) the tissue injured/damaged/impaired by said radiation-induced pathology and/or migrate toward and integrate the intestine tissue injured/damaged/impaired by said pathology of the intestine, as supported by the experimental data.

Preferably, the Muse cells:
repair/restore/regenerate at least partly (preferably at least 30%, more preferably at least 40%, more preferably at least 50%, more preferably at least 60%, more preferably at least 70%, more preferably at least 80%, more preferably at least 90%) the tissue injured/damaged/impaired by said radiation-induced pathology and/or the intestine tissue injured/damaged/impaired by said pathology of the intestine (in a subject administered with the Muse cells, compared to a subject not administered with the Muse cells); and/or
maintain/preserve at least partly (preferably at least 30%, more preferably at least 40%, more preferably at least 50%, more preferably at least 60%, more preferably at least 70%, more preferably at least 80%, more preferably at least 90%) the integrity of the tissue exposed to the radiations and/or the intestine tissue affected by said pathology of the intestine (in a subject administered with the human Muse cells, compared to a subject not administered with the Muse cells).

The data demonstrate that Muse cells integrate the injured tissue, prevent lethality, and maintain tissue integrity. In addition, the results show that that Muse cells injection rapidly (and transiently) enhance MCP-1 production that is associated with an early recruitment of monocytes into the lamina propria. In addition, Muse cells treatment is associated with the orientation of macrophages towards a M2 phenotype. These two features might contribute to intestinal tissue regeneration.

Thus, in a preferred embodiment, in a subject in need thereof, administered with the Muse cells, any of:
a) survival rates increase;
b) weight loss reduces;
c) levels of expression of the gene encoding Monocyte-Chemoattractant protein-1 (MCP1) (transiently) increase (by cells of the tissue injured/damaged/impaired by said radiation-induced pathology and/or of the intestine tissue injured/damaged/impaired by said pathology of the intestine);
d) level/number/quantity and/or percentage of Ly6C^{hi} monocytes population (transiently) increases (in the tissue injured/damaged/impaired by said radiation-induced pathology and/or in the intestine tissue injured/damaged/impaired by said pathology of the intestine);
e) level/number/quantity and/or percentage of CD68⁺CD206⁺ (M2-like) macrophages (transiently) increases (in the tissue injured/damaged/impaired by said radiation-induced pathology and/or in the intestine tissue injured/damaged/impaired by said pathology of the intestine); and
f) Any combination thereof;
(preferably compared to a subject not administered with the Muse cells).

Preferably, administration of the Muse cells to a subject in need thereof results in any of:
a) increased survival rates (preferably administration of the Muse cells increases survival rate);
b) reduced weight loss (preferably administration of the Muse cells reduces weight loss);
c) (transient) increased levels of expression of the gene encoding Monocyte-Chemoattractant protein-1 (MCP1) (preferably administration of the Muse cells transiently increases levels of expression of the gene encoding MCP1) (by cells of the tissue injured/damaged/impaired by said radiation-induced pathology and/or of the intestine tissue injured/damaged/impaired by said pathology of the intestine);
d) (transient) increased level/number/quantity and/or percentage of Ly6C^{hi} monocytes population (preferably administration of the Muse cells transiently increases level/number/quantity and/or percentage of Ly6C^{hi} monocytes population) (in the tissue injured/damaged/impaired by said radiation-induced pathology and/or in the intestine tissue injured/damaged/impaired by said pathology of the intestine);
e) (transient) increased level/number/quantity and/or percentage of CD68⁺CD206⁺ (M2-like) macrophages (preferably administration of the Muse cells transiently increases level/number/quantity and/or percentage of CD68⁺CD206⁺ (M2-like) macrophages) (in the tissue injured/damaged/impaired by said radiation-induced pathology and/or in the intestine tissue injured/damaged/impaired by said pathology of the intestine); and
f) Any combination thereof;
in the subject administered with the Muse cells, preferably compared to a subject not administered with the Muse cells.

Alternatively or in combination, in preferred embodiment, any one of:
a) increased survival rate;
b) reduced weight loss;
c) (transient) increased levels of expression of the gene encoding Monocyte-Chemoattractant protein-1 (MCP1) (by cells of the tissue injured/damaged/impaired by said radiation-induced pathology and/or of the intestine tissue injured/damaged/impaired by said pathology of the intestine);
d) (transient) increased level/number/quantity and/or percentage of Ly6C^{hi} monocytes population (in the tissue injured/damaged/impaired by said radiation-induced pathology and/or in the intestine tissue injured/damaged/impaired by said pathology of the intestine);
e) (transient) increased level/number/quantity and/or percentage of CD68⁺CD206⁺ (M2-like) macrophages (in the tissue injured/damaged/impaired by said radiation-induced pathology and/or in the intestine tissue injured/damaged/impaired by said pathology of the intestine); and
f) any combination thereof;
is/are observed in a subject in need thereof, administered with the Muse cells, preferably compared to a subject not administered with the Muse cells.

The results demonstrate that Muse cells activate the IL6/Stat3 signalling pathway in Paneth cells and increased their proliferation.

Thus, in a preferred embodiment, when the pathology is selected from the group consisting of radiation-induced intestine lesion, radiation-induced gastrointestinal syndrome (GIS), colitis, ulcer of the intestine, inflammatory bowel disease (such as ulcerative colitis (UC), Crohn's disease (CD)), cancer of the intestine, lesion of the intestine, and any combination thereof;
in a subject in need thereof, administered with the Muse cells, any of:
a) the crypt-villi structures of the intestine tissue are repaired/restored/regenerated//maintained/preserved, at least partly (preferably at least 30%, more preferably at least 40%, more preferably at least 50%, more preferably at least 60%, more preferably at least 70%, more preferably at least 80%, more preferably at least 90%);
b) the clonogenic crypt count increases in the intestine tissue;
c) the permeability of mucosal barrier of the intestine tissue is repaired/restored/regenerated//maintained/preserved, at least partly (preferably at least 30%, more preferably at least 40%, more preferably at least 50%, more preferably at least 60%, more preferably at least 70%, more preferably at least 80%, more preferably at least 90%);
d) the levels of expression of the gene encoding Zonula occludens-1 (ZO-1) are repaired/restored/regenerated//maintained/preserved in the cytomembrane of epithelial cells in the villi of the intestine tissue, at least partly (preferably at least 30%, more preferably at least 40%, more preferably at least 50%, more preferably at least 60%, more preferably at least 70%, more preferably at least 80%, more preferably at least 90%);
e) the levels of expression of the gene encoding Epithelial cell adhesion molecule (EpCAM) by cells of the intestine tissue is repaired/restored/regenerated//maintained/preserved, at least partly (preferably at least 30%, more preferably at least 40%, more preferably at least 50%, more preferably at least 60%, more preferably at least 70%, more preferably at least 80%, more preferably at least 90%);
f) the epithelium of the intestine tissue is repaired/restored/regenerated/maintained/preserved, at least partly (preferably at least 30%, more preferably at least 40%, more preferably at least 50%, more preferably at least 60%, more preferably at least 70%, more preferably at least 80%, more preferably at least 90%);
g) the levels/numbers/quantities and/or the percentages of intestinal crypt stem cells and/or Paneth cells increases in the intestine, and the levels/numbers/quantities and/or the percentages of Ki67⁺CD24⁺ cells preferably increase in the intestine tissue;
h) the levels/numbers/quantities and/or the percentages of IL6 increases (preferably the levels/numbers/quantities and/or the percentages of IL6 increase in the intestine tissue, more preferably the secretion of IL6 increases in the intestine tissue), and the levels/numbers/quantities and/or the percentages of Paneth cells preferably increase in the intestine tissue; and
i) any combination thereof;

(preferably compared to a subject not administered with the Muse cells).

Additionally, in a subject in need thereof, administered with the Muse cells, the ratio of phospho-Stat3 (p-Stat3)/Stat3 (Signal transducer and activator of transcription 3) may preferably increase (preferably at least 1.5-fold, preferably at least 2-fold, compared to a subject not administered with the Muse cells) (preferably in the intestine tissue).

Preferably, when the pathology is selected from the group consisting of radiation-induced intestine lesion, radiation-induced gastrointestinal syndrome (GIS), colitis, ulcer of the intestine, inflammatory bowel disease (such as ulcerative colitis (UC), Crohn's disease (CD)), cancer of the intestine, lesion of the intestine, and any combination thereof;
administration of the Muse cells to a subject in need thereof results in any of:
a) repaired/restored/regenerated/maintained/preserved crypt-villi structures of the intestine tissue (preferably administration of the Muse cells repairs/restores/regenerates/maintains/preserves crypt-villi structures of the intestine tissue), at least partly (preferably at least 30%, more preferably at least 40%, more preferably at least 50%, more preferably at least 60%, more preferably at least 70%, more preferably at least 80%, more preferably at least 90%);
b) increased clonogenic crypt count in the intestine tissue (preferably administration of the Muse cells increases clonogenic crypt count in the intestine);
c) repaired/restored/regenerated/maintained/preserved permeability of mucosal barrier of the intestine tissue (preferably administration of the Muse cells repairs/restores/regenerates/maintains/preserves permeability of mucosal barrier of the intestine tissue), at least partly (preferably at least 30%, more preferably at least 40%, more preferably at least 50%, more preferably at least 60%, more preferably at least 70%, more preferably at least 80%, more preferably at least 90%);
d) repaired/restored/regenerated/maintained/preserved levels of expression of the gene encoding Zonula occludens-1 (ZO-1), preferably in the cytomembrane of epithelial cells in the villi of the intestine tissue (preferably administration of the Muse cells repairs/restores/regenerates/maintains/preserves levels of expression of the gene encoding ZO-1), at least partly (preferably at least 30%, more preferably at least 40%, more preferably at least 50%, more preferably at least 60%, more preferably at least 70%, more preferably at least 80%, more preferably at least 90%);
e) repaired/restored/regenerated/maintained/preserved levels of expression of the gene encoding Epithelial cell adhesion molecule (EpCAM) by cells of the intestine tissue (preferably administration of the Muse cells repairs/restores/regenerates/maintains/preserves levels of expression of the gene encoding EpCAM by cells of the intestine tissue), at least partly (preferably at least 30%, more preferably at least 40%, more preferably at least 50%, more preferably at least 60%, more preferably at least 70%, more preferably at least 80%, more preferably at least 90%);
f) repaired/restored/regenerated/maintained/preserved epithelium of the intestine tissue (preferably administration of the Muse cells repairs/restores/regenerates/maintains/preserves the epithelium of the intestine tissue), at least partly (preferably at least 30%, more preferably at least 40%, more preferably at least 50%, more preferably at least 60%, more preferably at least 70%, more preferably at least 80%, more preferably at least 90%);
g) increased levels/numbers/quantities and/or percentages of intestinal crypt stem cells and/or Paneth cells in the intestine (preferably administration of the Muse cells increases levels/numbers/quantities and/or percentages of intestinal crypt stem cells and/or Paneth cells in the intestine), and preferably increased levels/numbers/quantities and/or percentages of Ki67⁺CD24⁺ cells in the intestine tissue (preferably administration of the Muse cells increases levels/numbers/quantities and/or percentages of Ki67⁺CD24⁺ cells in the intestine tissue);
h) increased levels/numbers/quantities and/or percentages of IL6 (preferably increased levels/numbers/quantities and/or percentages of IL6 in the intestine tissue, more preferably increased secretion of IL6 in the intestine tissue) (preferably administration of the Muse cells increases levels/numbers/quantities and/or percentages of IL6, preferably in the intestine tissue), and preferably increased levels/numbers/quantities and/or percentages of Paneth cells in the intestine tissue (preferably administration of the Muse cells increases levels/numbers/quantities and/or percentages of Paneth cells in the intestine tissue; and
i) any combination thereof;
in the subject administered with the Muse cells, preferably compared to a subject not administered with the Muse cells.

Additionally, administration of the Muse cells to a subject in need thereof may preferably results in an increased p-Stat3/Stat3 ratio (preferably in the intestine tissue); preferably administration of the Muse cells increases p-Stat3/Stat3 ratio (preferably in the intestine tissue); (preferably at least 1.5-fold, preferably at least 2-fold, compared to a subject not administered with the Muse cells).

Alternatively or in combination, in preferred embodiment, when the pathology is selected from the group consisting of radiation-induced intestine lesion, radiation-induced gastrointestinal syndrome (GIS), colitis, ulcer of the intestine, inflammatory bowel disease (such as ulcerative colitis (UC), Crohn's disease (CD)), cancer of the intestine, lesion of the intestine, and any combination thereof; any one of:
a) repaired/restored/regenerated/maintained/preserved crypt-villi structures of the intestine tissue at least partly (preferably at least 30%, more preferably at least 40%, more preferably at least 50%, more preferably at least 60%, more preferably at least 70%, more preferably at least 80%, more preferably at least 90%);
b) increased clonogenic crypt count in the intestine tissue;
c) repaired/restored/regenerated/maintained/preserved permeability of mucosal barrier of the intestine tissue at least partly (preferably at least 30%, more preferably at least 40%, more preferably at least 50%, more preferably at least 60%, more preferably at least 70%, more preferably at least 80%, more preferably at least 90%);
d) repaired/restored/regenerated/maintained/preserved levels of expression of the gene encoding Zonula occludens-1 (ZO-1) (preferably in the cytomembrane of epithelial cells in the villi of the intestine tissue), at least partly (preferably at least 30%, more preferably at least 40%, more preferably at least 50%, more preferably at least 60%, more preferably at least 70%, more preferably at least 80%, more preferably at least 90%);
e) repaired/restored/regenerated/maintained/preserved levels of expression of the gene encoding Epithelial cell adhesion molecule (EpCAM) by cells of the intestine tissue, at least partly (preferably at least 30%, more preferably at least 40%, more preferably at least 50%, more preferably at least 60%, more preferably at least 70%, more preferably at least 80%, more preferably at least 90%);
f) repaired/restored/regenerated/maintained/preserved epithelium of the intestine tissue, at least partly (preferably at least 30%, more preferably at least 40%, more preferably at least 50%, more preferably at least 60%, more preferably at least 70%, more preferably at least 80%, more preferably at least 90%);
g) increased levels/numbers/quantities and/or percentages of intestinal crypt stem cells and/or Paneth cells in the intestine, and preferably increased levels/numbers/quantities and/or percentages of Ki67⁺CD24⁺ cells in the intestine tissue;
h) increased levels/numbers/quantities and/or percentages of IL6 (preferably increased levels/numbers/quantities and/or percentages of IL6 in the intestine tissue, more preferably increased secretion of IL6 in the intestine tissue), and preferably increased levels/numbers/quantities and/or percentages of Paneth cells in the intestine tissue; and
i) any combination thereof;
   is/are observed in a subject in need thereof, administered with the Muse cells, preferably compared to a subject not administered with the Muse cells.

Additionally, increased p-Stat3/Stat3 ratio (preferably in the intestine tissue) is/are observed in a subject in need thereof, administered with the Muse cells, preferably compared to a subject not administered with the Muse cells (preferably at least 1.5-fold, preferably at least 2-fold, compared to a subject not administered with the Muse cells).

Advantageously, an amount of at least 50000 Muse cells is administered to a subject in need thereof, preferably an amount of Muse cells ranging from 50000 to 15 million Muse cells, more preferably from 100000 to 12 million Muse cells, more preferably from 500000 to 10 million Muse cells, more preferably from 1 million to 5 million Muse cells.

The Muse cells are preferably administered to a subject in need thereof by injection, preferably via intravenous route. Thus, the Muse cells are preferably formulated for an administration by injection, preferably via intravenous route.

The Muse cells are preferably administered to a subject in need thereof:
(i) at an early stage after the onset of said pathology, preferably within a period of time of 6 months after the onset of the first symptoms associated with said pathology, preferably within a period of time ranging from 1 day to 6 months after the onset of the first symptoms associated with said pathology, more preferably from 1 week to 5 months, more preferably from 2 weeks to 4 months, more preferably from 3 weeks to 3 months, more preferably from 4 weeks to 2 months, even more preferably within the first month after the onset of the first symptoms associated with said pathology, even more preferably within the first week after the onset of the first symptoms associated with said pathology, in particular when the pathology is a radiation-induced pathology; and/or
(ii) at a later stage after the onset of said pathology, preferably after a period of time of 6 months after the onset of the first symptoms associated with said pathology, more preferably within a period of time ranging from 6 months to 5 years, more preferably from 7 months to 4 years, more preferably from 8 months to 3 years, more preferably from 9 months to 2 years, more preferably from 10 months to 14 months, after the onset of the first symptoms associated with said pathology;
   in particular when the pathology is a pathology of the intestine, especially a chronic pathology of the intestine.

When the pathology is a radiation-induced pathology, the Muse cells are preferably administered to a subject in need thereof at an early stage after the onset of said pathology, preferably within a period of time ranging from 1 h to 2 weeks after the onset of the first symptoms associated with said pathology, more preferably from 12 h to 1 week, more preferably from 1 to 5 days, more preferably from 2 to 3 days, even more preferably within the first 2 days after the onset of the first symptoms associated with said pathology.

When the pathology is a chronic pathology, the Muse cells are preferably administered to a subject in need thereof more than once, preferably in a regular way. Accordingly, the Muse cells are preferably administered at least once every 3 years, more preferably at least once every 2 years, more preferably at least once every year, more preferably at least once every 6 months, more preferably at least once every 3 months, more preferably at least once every 2 months, more preferably at least once every month, more preferably at least once every 2 weeks, more preferably at least once every week.

The Inventors showed that cryopreservation does not affect Muse cells properties and viability. Indeed, viability of cryopreserved Muse cells is superior to 90%. Importantly, treatment of radiation-induced pathologies was as efficient with cryopreserved Muse cells than with fresh Muse cells. Thus, the Muse cells are advantageously cryopreserved.

Methods and means for cryopreserving cells are well known in the art. The Muse cells may be typically be frozen for 12 h to 36h (preferably for about 24 h) at approximately-80° C. (for instance in isopropanol freezing container). Long-term storage may then be performed into liquid nitrogen.

Compositions Comprising Muse Cells for Use for Treating a Pathology Selected from Radiation-Induced Pathologies and Pathologies of the Intestine

In the context of the present invention, the Inventors surprisingly found that administering a population of cells, called Multilineage-differentiating stress-enduring (Muse) cells, results in efficient, rapid and durable treatment of radiation-induced pathologies and pathologies of the intestine.

Accordingly, the present invention concerns a composition comprising, or consisting essentially of, or consisting of, Muse cells for use for treating a pathology selected from radiation-induced pathologies and pathologies of the intestine, preferably wherein the Muse cells are SSEA-3⁺CD105⁺.

The present invention also concerns the use of a composition comprising, or consisting essentially of, or consisting of, Muse cells, for treating a pathology selected from radiation-induced pathologies and pathologies of the intestine (preferably comprising the administration of a therapeutically effective amount of said composition to a subject in need thereof), wherein the Muse cells are preferably SSEA-3⁺CD105⁺.

The present invention also concerns the use of a composition comprising, or consisting essentially of, or consisting of, Muse cells, for manufacturing a medicament for treating a pathology selected from radiation-induced pathologies and pathologies of the intestine, wherein the Muse cells are preferably SSEA-3⁺CD105⁺.

The present invention also provides a method for treating a pathology selected from radiation-induced pathologies and pathologies of the intestine, comprising the administration of a therapeutically effective amount of a composition comprising, or consisting essentially of, or consisting of, Muse cells, to a subject in need thereof, wherein the Muse cells are preferably SSEA-3⁺CD105⁺.

The Muse cells are preferably mammal Muse cells, more preferably human Muse cells. The present invention thus relates to a composition comprising, or consisting essentially of, or consisting of, human Muse cells, for use for treating a pathology selected from radiation-induced pathologies and pathologies of the intestine, wherein the Muse cells are preferably SSEA-3⁺CD105⁺.

The Muse cells, the pathologies, the administration, are preferably as described above in relation the Muse cells for use for treating a pathology selected from radiation-induced pathologies and pathologies of the intestine (in the section entitled “Muse cells for use for treating a pathology selected from radiation-induced pathologies and pathologies of the intestine-Methods of treatment”, above).

In a preferred embodiment, at least 70% of the cells comprised in the composition are SSEA-3⁺CD105⁺, preferably at least 80% of the cells comprised in the composition are SSEA-3⁺CD105⁺, more preferably at least 90% of the cells comprised in the composition are SSEA-3⁺CD105⁺, more preferably at least 95% of the cells comprised in the composition are SSEA-3⁺CD105⁺, more preferably at least 98% of the cells comprised in the composition are SSEA-3⁺CD105⁺, more preferably at least 99% of the cells comprised in the composition are SSEA-3⁺CD105⁺, even more preferably 100% of the cells comprised in the composition are SSEA-3⁺CD105⁺.

The composition advantageously comprises, or consists essentially of, or consists of, a therapeutically effective amount of Muse cells. The composition advantageously comprises, or consists essentially of, or consists of, an amount of at least 50000 Muse cells, preferably an amount of Muse cells ranging from 50000 to 15 million cells, more preferably from 100000 to 12 million Muse cells, more preferably from 500000 to 10 million cells, more preferably from 1 million to 5 million Muse cells.

The experimental data show that the administration of 50000 cells is sufficient to obtain the beneficial effect. Thus, in an advantageous embodiment, the composition comprises, or consists essentially of, or consists of, an amount of 1 000 000 Muse cells or less, preferably an amount of 800000 Muse cells or less, preferably an amount of 500000 Muse cells or less, more preferably an amount of 400000 Muse cells or less, more preferably an amount of 300000 Muse cells or less, more preferably an amount of 200000 Muse cells or less, more preferably an amount of 100000 Muse cells or less, more preferably an amount of 90000 Muse cells or less, more preferably an amount of 80000 Muse cells or less, more preferably an amount of 70000 Muse cells or less, more preferably an amount of 60000 Muse cells or less, more preferably an amount of 55000 Muse cells or less, more preferably an amount of about 50000 Muse cells.

The composition may further comprise one or more pharmaceutically acceptable vehicle(s).

The composition is preferably administered to a subject in need thereof by injection, preferably via intravenous route. Thus, the composition is preferably formulated for an administration by injection, preferably via intravenous route.

The composition is preferably administered to a subject in need thereof:
(i) at an early stage after the onset of said pathology, preferably within a period of time of 6 months after the onset of the first symptoms associated with said pathology, preferably within a period of time ranging from 1 day to 6 months after the onset of the first symptoms associated with said pathology, more preferably from 1 week to 5 months, more preferably from 2 weeks to 4 months, more preferably from 3 weeks to 3 months, more preferably from 4 weeks to 2 months, even more preferably within the first month after the onset of the first symptoms associated with said pathology, even more preferably within the first week after the onset of the first symptoms associated with said pathology,
   in particular when the pathology is a radiation-induced pathology; and/or
(ii) at a later stage after the onset of said pathology, preferably after a period of time of 6 months after the onset of the first symptoms associated with said pathology, more preferably within a period of time ranging from 6 months to 5 years, more preferably from 7 months to 4 years, more preferably from 8 months to 3 years, more preferably from 9 months to 2 years, more preferably from 10 months to 14 months, after the onset of the first symptoms associated with said pathology;
   in particular when the pathology is a pathology of the intestine, especially a chronic pathology of the intestine.

When the pathology is a radiation-induced pathology, the composition is preferably administered to a subject in need thereof at an early stage after the onset of said pathology, preferably within a period of time ranging from 1 h to 2 weeks after the onset of the first symptoms associated with said pathology, more preferably from 12 h to 1 week, more preferably from 1 to 5 days, more preferably from 2 to 3 days, even more preferably within the first 2 days after the onset of the first symptoms associated with said pathology.

When the pathology is a chronic pathology, the composition is preferably administered to a subject in need thereof more than once, preferably in a regular way. Accordingly, the composition is preferably administered at least once every 3 years, more preferably at least once every 2 years, more preferably at least once every year, more preferably at least once every 6 months, more preferably at least once every 3 months, more preferably at least once every 2 months, more preferably at least once every month, more preferably at least once every 2 weeks, more preferably at least once every week.

In an advantageous embodiment, the Muse cells of the composition are capable to migrate toward and integrate (or migrate toward and integrate) the tissue injured/damaged/impaired by said radiation-induced pathology and/or migrate toward and integrate the intestine tissue injured/damaged/impaired by said pathology of the intestine, as supported by the experimental data.

Preferably, the Muse cells:
repair/restore/regenerate at least partly (preferably at least 30%, more preferably at least 40%, more preferably at least 50%, more preferably at least 60%, more preferably at least 70%, more preferably at least 80%, more preferably at least 90%) the tissue injured/damaged/impaired by said radiation-induced pathology and/or the intestine tissue injured/damaged/impaired by said pathology of the intestine (in a subject administered with the composition, compared to a subject not administered with the composition); and/or
maintain/preserve at least partly (preferably at least 30%, more preferably at least 40%, more preferably at least 50%, more preferably at least 60%, more preferably at least 70%, more preferably at least 80%, more preferably at least 90%) the integrity of the tissue exposed to the radiations and/or the intestine tissue affected by said pathology of the intestine (in a subject administered with the composition, compared to a subject not administered with the composition).

The data demonstrate that Muse cells integrate the injured tissue, prevent lethality, and maintain tissue integrity. In addition, the results show that that Muse cells injection rapidly (and transiently) enhance MCP-1 production that is associated with an early recruitment of monocytes into the Lamina propria. In addition, Muse cells treatment is associated with the orientation of macrophages towards a M2 phenotype. These two features might contribute to intestinal tissue regeneration.

Thus, in a preferred embodiment, in a subject in need thereof, administered with the composition, any of:
a) survival rates increase;
b) weight loss reduces;
c) levels of expression of the gene encoding Monocyte-Chemoattractant protein-1 (MCP1) (transiently) increase (by cells of the tissue injured/damaged/impaired by said radiation-induced pathology and/or of the intestine tissue injured/damaged/impaired by said pathology of the intestine);
d) level/number/quantity and/or percentage of Ly6Chi monocytes population (transiently) increases (in the tissue injured/damaged/impaired by said radiation-induced pathology and/or in the intestine tissue injured/damaged/impaired by said pathology of the intestine);
e) level/number/quantity and/or percentage of CD68⁺CD206⁺ (M2-like) macrophages (transiently) increases (in the tissue injured/damaged/impaired by said radiation-induced pathology and/or in the intestine tissue injured/damaged/impaired by said pathology of the intestine); and
f) Any combination thereof;
   (preferably compared to a subject not administered with the composition).

Preferably, administration of the composition to a subject in need thereof:
a) results in increased survival rates (preferably administration of the composition increases survival rate);
b) results in reduced weight loss (preferably administration of the composition reduces weight loss);
c) results in (transient) increased levels of expression of the gene encoding Monocyte-Chemoattractant protein-1 (MCP1) (preferably administration of the composition transiently increases levels of expression of the gene encoding MCP1) (by cells of the tissue injured/damaged/impaired by said radiation-induced pathology and/or of the intestine tissue injured/damaged/impaired by said pathology of the intestine);
d) results in (transient) increased level/number/quantity and/or percentage of Ly6Chi monocytes population (preferably administration of the composition transiently increases level/number/quantity and/or percentage of Ly6Chi monocytes population) (in the tissue injured/damaged/impaired by said radiation-induced pathology and/or in the intestine tissue injured/damaged/impaired by said pathology of the intestine);
e) results in (transient) increased level/number/quantity and/or percentage of CD68⁺CD206⁺ (M2-like) macrophages (preferably administration of the composition transiently increases level/number/quantity and/or percentage of CD68⁺CD206⁺ (M2-like) macrophages) (in the tissue injured/damaged/impaired by said radiation-induced pathology and/or in the intestine tissue injured/damaged/impaired by said pathology of the intestine); and
f) Any combination thereof;

in the subject administered with the composition, preferably compared to a subject not administered with the composition.

Alternatively or in combination, in preferred embodiment, any one of:
a) increased survival rate;
b) reduced weight loss;
c) (transient) increased levels of expression of the gene encoding Monocyte-Chemoattractant protein-1 (MCP1) (by cells of the tissue injured/damaged/impaired by said radiation-induced pathology and/or of the intestine tissue injured/damaged/impaired by said pathology of the intestine);
d) (transient) increased level/number/quantity and/or percentage of Ly6C^{hi} monocytes population (in the tissue injured/damaged/impaired by said radiation-induced pathology and/or in the intestine tissue injured/damaged/impaired by said pathology of the intestine);
e) (transient) increased level/number/quantity and/or percentage of CD68⁺CD206⁺ (M2-like) macrophages (in the tissue injured/damaged/impaired by said radiation-induced pathology and/or in the intestine tissue injured/damaged/impaired by said pathology of the intestine); and
f) any combination thereof;
is/are observed in a subject in need thereof, administered with the composition, preferably compared to a subject not administered with the composition.

In a preferred embodiment, when the pathology is selected from the group consisting of radiation-induced intestine lesion, radiation-induced gastrointestinal syndrome (GIS), colitis, ulcer of the intestine, inflammatory bowel disease (such as ulcerative colitis (UC), Crohn's disease (CD)), cancer of the intestine, lesion of the intestine, and any combination thereof;

in a subject in need thereof, administered with the composition, any of:
a) the crypt-villi structures of the intestine tissue are repaired/restored/regenerated/maintained/preserved, at least partly (preferably at least 30%, more preferably at least 40%, more preferably at least 50%, more preferably at least 60%, more preferably at least 70%, more preferably at least 80%, more preferably at least 90%);
b) the clonogenic crypt count increases in the intestine tissue;
c) the permeability of mucosal barrier of the intestine tissue is repaired/restored/regenerated/maintained/preserved, at least partly (preferably at least 30%, more preferably at least 40%, more preferably at least 50%, more preferably at least 60%, more preferably at least 70%, more preferably at least 80%, more preferably at least 90%);
d) the levels of expression of the gene encoding Zonula occludens-1 (ZO-1) is repaired/restored/regenerated/maintained/preserved in the cytomembrane of epithelial cells in the villi of the intestine tissue, at least partly (preferably at least 30%, more preferably at least 40%, more preferably at least 50%, more preferably at least 60%, more preferably at least 70%, more preferably at least 80%, more preferably at least 90%);
e) the levels of expression of the gene encoding Epithelial cell adhesion molecule (EpCAM) by cells of the intestine tissue is repaired/restored/regenerated/maintained/preserved, at least partly (preferably at least 30%, more preferably at least 40%, more preferably at least 50%, more preferably at least 60%, more preferably at least 70%, more preferably at least 80%, more preferably at least 90%);
f) the epithelium of the intestine tissue is repaired/restored/regenerated/maintained/preserved, at least partly (preferably at least 30%, more preferably at least 40%, more preferably at least 50%, more preferably at least 60%, more preferably at least 70%, more preferably at least 80%, more preferably at least 90%);
g) the levels/numbers/quantities and/or the percentages of intestinal crypt stem cells and/or Paneth cells increases in the intestine, and the levels/numbers/quantities and/or the percentages of Ki67⁺CD24⁺ cells preferably increase in the intestine tissue;
h) the levels/numbers/quantities and/or the percentages of IL6 increases (preferably the levels/numbers/quantities and/or the percentages of IL6 increases in the intestine tissue, more preferably the secretion of IL6 increases in the intestine tissue), and the levels/numbers/quantities and/or the percentages of Paneth cells preferably increase in the intestine tissue; and
i) any combination thereof;
   (preferably compared to a subject not administered with the composition).

Additionally, in a subject in need thereof, administered with the composition, the ratio of phospho-Stat3 (p-Stat3)/Stat3 (Signal transducer and activator of transcription 3) may preferably increase (preferably at least 1.5-fold, preferably at least 2-fold, compared to a subject not administered with the composition) (preferably in the intestine tissue).

Preferably, when the pathology is selected from the group consisting of radiation-induced intestine lesion, radiation-induced gastrointestinal syndrome (GIS), colitis, ulcer of the intestine, inflammatory bowel disease (such as ulcerative colitis (UC), Crohn's disease (CD)), cancer of the intestine, lesion of the intestine, and any combination thereof;

administration of the composition to a subject in need thereof results in any of:
a) repaired/restored/regenerated/maintained/preserved crypt-villi structures of the intestine tissue (preferably administration of the Muse cells repairs/restores/regenerates/maintains/preserves crypt-villi structures of the intestine tissue), at least partly (preferably at least 30%, more preferably at least 40%, more preferably at least 50%, more preferably at least 60%, more preferably at least 70%, more preferably at least 80%, more preferably at least 90%);
b) increased clonogenic crypt count in the intestine tissue (preferably administration of the Muse cells increases clonogenic crypt count in the intestine);
c) repaired/restored/regenerated/maintained/preserved permeability of mucosal barrier of the intestine tissue (preferably administration of the Muse cells repairs/restores/regenerates/maintains/preserves permeability of mucosal barrier of the intestine tissue), at least partly (preferably at least 30%, more preferably at least 40%, more preferably at least 50%, more preferably at least 60%, more preferably at least 70%, more preferably at least 80%, more preferably at least 90%);
d) repaired/restored/regenerated/maintained/preserved levels of expression of the gene encoding Zonula occludens-1 (ZO-1), preferably in the cytomembrane of epithelial cells in the villi of the intestine tissue (preferably administration of the Muse cells repairs/restores/regenerates/maintains/preserves levels of expression of the gene encoding ZO-1), at least partly (preferably at least 30%, more preferably at least 40%, more preferably at least 50%, more preferably at least 60%, more preferably at least 70%, more preferably at least 80%, more preferably at least 90%);
e) repaired/restored/regenerated/maintained/preserved levels of expression of the gene encoding Epithelial cell adhesion molecule (EpCAM) by cells of the intestine tissue (preferably administration of the Muse cells repairs/restores/regenerates/maintains/preserves levels of expression of the gene encoding EpCAM by cells of the intestine tissue), at least partly (preferably at least 30%, more preferably at least 40%, more preferably at least 50%, more preferably at least 60%, more preferably at least 70%, more preferably at least 80%, more preferably at least 90%);
f) repaired/restored/regenerated/maintained/preserved epithelium of the intestine tissue (preferably administration of the Muse cells repairs/restores/regenerates/maintains/preserves the epithelium of the intestine tissue), at least partly (preferably at least 30%, more preferably at least 40%, more preferably at least 50%, more preferably at least 60%, more preferably at least 70%, more preferably at least 80%, more preferably at least 90%);
g) increased levels/numbers/quantities and/or percentages of intestinal crypt stem cells and/or Paneth cells in the intestine (preferably administration of the Muse cells increases levels/numbers/quantities and/or percentages of intestinal crypt stem cells and/or Paneth cells in the intestine), and preferably increased levels/numbers/quantities and/or percentages of Ki67⁺CD24⁺ cells in the intestine tissue (preferably administration of the Muse cells increases levels/numbers/quantities and/or percentages of Ki67⁺CD24⁺ cells in the intestine tissue);
h) increased levels/numbers/quantities and/or percentages of IL6 (preferably increased levels/numbers/quantities and/or percentages of IL6 in the intestine tissue, more preferably increased secretion of IL6 in the intestine tissue) (preferably administration of the Muse cells increases levels/numbers/quantities and/or percentages of IL6, preferably in the intestine tissue), and preferably increased levels/numbers/quantities and/or percentages of Paneth cells in the intestine tissue (preferably administration of the Muse cells increases levels/numbers/quantities and/or percentages of Paneth cells in the intestine tissue; and
i) any combination thereof;
   in the subject administered with the composition, preferably compared to a subject not administered with the composition.

Additionally, administration of the composition to a subject in need thereof may preferably results in an increased p-Stat3/Stat3 ratio (preferably in the intestine tissue); preferably administration of the composition increases p-Stat3/Stat3 ratio (preferably in the intestine tissue); (preferably at least 1.5-fold, preferably at least 2-fold, compared to a subject not administered with the composition).

Alternatively or in combination, in preferred embodiment, when the pathology is selected from the group consisting of radiation-induced intestine lesion, radiation-induced gastrointestinal syndrome (GIS), colitis, ulcer of the intestine, inflammatory bowel disease (such as ulcerative colitis (UC), Crohn's disease (CD)), cancer of the intestine, lesion of the intestine, and any combination thereof; any one of:
a) repaired/restored/regenerated/maintained/preserved crypt-villi structures of the intestine tissue at least partly (preferably at least 30%, more preferably at least 40%, more preferably at least 50%, more preferably at least 60%, more preferably at least 70%, more preferably at least 80%, more preferably at least 90%);
b) increased clonogenic crypt count in the intestine tissue;
c) repaired/restored/regenerated/maintained/preserved permeability of mucosal barrier of the intestine tissue at least partly (preferably at least 30%, more preferably at least 40%, more preferably at least 50%, more preferably at least 60%, more preferably at least 70%, more preferably at least 80%, more preferably at least 90%);
d) repaired/restored/regenerated/maintained/preserved levels of expression of the gene encoding Zonula occludens-1 (ZO-1) (preferably in the cytomembrane of epithelial cells in the villi of the intestine tissue), at least partly (preferably at least 30%, more preferably at least 40%, more preferably at least 50%, more preferably at least 60%, more preferably at least 70%, more preferably at least 80%, more preferably at least 90%);
e) repaired/restored/regenerated/maintained/preserved levels of expression of the gene encoding Epithelial cell adhesion molecule (EpCAM) by cells of the intestine tissue, at least partly (preferably at least 30%, more preferably at least 40%, more preferably at least 50%, more preferably at least 60%, more preferably at least 70%, more preferably at least 80%, more preferably at least 90%);
f) repaired/restored/regenerated/maintained/preserved epithelium of the intestine tissue, at least partly (preferably at least 30%, more preferably at least 40%, more preferably at least 50%, more preferably at least 60%, more preferably at least 70%, more preferably at least 80%, more preferably at least 90%);
g) increased levels/numbers/quantities and/or percentages of intestinal crypt stem cells and/or Paneth cells in the intestine, and preferably increased levels/numbers/quantities and/or percentages of Ki67⁺CD24⁺ cells in the intestine tissue;
h) increased levels/numbers/quantities and/or percentages of IL6 (preferably increased levels/numbers/quantities and/or percentages of IL6 in the intestine tissue, more preferably increased secretion of IL6 in the intestine tissue), and preferably increased levels/numbers/quantities and/or percentages of Paneth cells in the intestine tissue; and i) any combination thereof;
is/are observed in a subject in need thereof, administered with the composition, preferably compared to a subject not administered with the composition.

Additionally, increased p-Stat3/Stat3 ratio (preferably in the intestine tissue) is/are observed in a subject in need thereof, administered with the composition, preferably compared to a subject not administered with the composition (preferably at least 1.5-fold, preferably at least 2-fold, compared to a subject not administered with the composition).

### DESCRIPTION OF THE FIGURES

FIG. 1

(A) Representative flow cytometry plots showing the viability (Hoechst) and phenotype (CD45, CD44, CD105, CD90 and CD73) of WJ-MSC before culture amplification.

(B) Representative flow cytometry plots showing the percentage of SSEA-3⁺Muse cells obtained after amplification of BM-MSC or WJ-MSC at passage 7.

(C) Illustration of BM-Muse and WJ-Muse after culture in adherence condition (top) and BM-Muse and WJ-Muse clusters spontaneously obtained after culture in methylcellulose (bottom). (D) Volcano plot displaying the differential abundance of proteins in BM-Muse and WJ-Muse cells analysed by MS-based label-free quantitative proteomics. Right and left dots represent proteins found significantly enriched respectively in BM-Muse and WJ-Muse cells respectively (fold change ≥2 and p-value≤0.005, leading to a Benjamini-Hochberg FDR <1%).

(E) Comparative analysis of common biological processes occurring in BM-Muse and WJ-Muse cells, identified by proteomic analysis.

FIG. 2. WJ-Muse have biological advantages in comparison to BM-Muse

(A) Proportion of Muse cells among the BM-MSC or the WJ-MSC population, before and after amplification until passage 7 of culture.

(B) Comparison of the cumulative population doubling levels (CPDL) of BM-MSC and WJ-MSC between passage 3 and passage 8 of culture. Data are represented with means±SEM. (C) Representative flow analysis of CD45 (hematopoietic marker) and CD105 (MSC marker) in BM-Muse and WJ-Muse cells.

(D) mRNA expression by RT-qPCR of Sox2, Nanog and Oct3/4 in WJ-Muse compared to BM-Muse. Data are represented with means±SEM, * p<0.05 (two-tailed Mann-Whitney U test).

(E) Adipogenic, osteogenic and epithelial differentiation of BM-Muse and WJ-Muse. Upper images: morphological features of BM-Muse and WJ-Muse are illustrated before and after differentiation. Adipocyte cells are stained with oil Red O; osteocyte cells are immunostained with osteocalcin (light grey) and counterstained with Dapi (dark grey); Lower histograms: epithelial differentiation was shown in BM-Muse and WJ-Muse with mRNA expression of cytokeratin18 (CK18) and occluding (OCLN) with or without retinoic acid treatment.

(F) Comparative analysis of biological processes occurring in BM-Muse and WJ-Muse cells, identified by proteomic analysis.

FIG. 3. WJ-Muse display immunosuppressive properties

(A) Left: Representative flow analysis of HLA-G5 (soluble human leukocyte antigen-G5 marker), HLA-G1 (membrane-bound human leukocyte antigen-G1 marker), HLA-DR (MHC Class II, human leukocyte antigen marker) and PD-L1 (human Programmed death-ligand 1) in naive WJ-Muse. IgG were used as reference controls. Right: Representative flow analysis of HLA-G1, HLA-DR and PD-L1 in TNFα/IFNγ-primed WJ-Muse compared to naive WJ-Muse. (B) Evaluation of IDO, Cox2, PD-L1 and TGFβ1 expression level by quantitative real time PCR, in TNFα/IFNγ-primed WJ-Muse compared to naive WJ-Muse. Data are represented with means±SEM, ** p<0.01; *** p<0.001; * **** p<0.0001 (unpaired Student's t-test).

(C) 105 human peripheral blood mononuclear cells (hPBMC) or murine spleen lymphocytes (mSL) were co-cultured with 104 WJ-Muse in order to evaluate their allogeneic or xenogeneic immune privilege. Frequency and proliferation of BrdU-labelled CD3⁺T-cells were analysed by flow cytometry and compared to hPBMC or mSL alone. Data are represented with means±SEM.

(D) Human peripheral blood mononuclear cells (hPBMC) or murine spleen lymphocytes (mSL) activated with concanavalin A were co-cultured with 5×10³, 10×10³ or 20×10³ WJ-Muse to determine their allogeneic and xenogeneic immunosuppressive potential respectively. Frequency (top) and proliferation (bottom) of human (left) or murine (right) BrdU-labelled CD3⁺T-cells were analyzed by flow cytometry and compared respectively with activated hPBMC or mSL alone. Data are represented with means±SEM, * p<0.05, ** p<0.01 (two-tailed MannWhitney U test).

(E) Arginase-1 (Arg1) and Nitric oxide synthase-2 (Nos2) mRNA expression were measured in bone marrow-derived macrophages (BMDM), which have been co-cultured for 7 days with WJ-Muse. Data are represented with mean±SEM.

FIG. 4. Muse improve survival by maintaining the intestinal integrity in GIS mouse model (A) Representative images of immunofluorescence showing Lysozyme-positive cells (light grey) in the small intestine of non-treated or Muse-treated mice at 7 days after irradiation. Integrated GFP+Muse (white) are identified with white arrows. Nuclei were counterstained with DAPI (dark grey).

(B) Kaplan-Meier survival analysis (left) and weight loss changes (right) for 29 days of 18 Gy abdominal exposed mice receiving or not a 50,000 MSC or 50,000 Muse-treatment 4 hours after irradiation. Statistical difference of survival between groups was determined by Log-rank (Mantel-Cox) test, with *p≤0.05 considered as significant. Weight data are represented with means±SEM. (C) Kaplan-Meier survival analysis (left) and weight loss changes (right) for 29 days of 18 Gy abdominal exposed mice receiving or not a 50,000 Muse-treatment 4 hours, 24 hours or 5 days after irradiation. Statistical difference of survival between groups was determined by Logrank (Mantel-Cox) test, with *p<0.05 considered as significant. Weight data are represented with means±SEM.

(D) Illustration (left), length (middle) and weight (right) of the small intestine of non-treated or Muse-treated mice 7 days after irradiation, compared to non-irradiated (NIR) control mice. Data are represented with means±SEM, * p<0.05; ns: not significant (two-tailed Mann-Whitney U test).

(E) Representative HE staining of small intestine of non-treated or Muse-treated mice at 1, 3.5 and 7 days after irradiation, compared to non-irradiated control mice.

(F) Histogram plots showing the percentage of surviving clonogenic crypts in small intestine of non-treated or Muse-treated mice at 3.5 days after irradiation, compared to non-irradiated control mice. Data are represented with means±SEM, * p<0.05; **** p<0.001 (two-tailed Mann-Whitney U test).

FIG. 5.

(A) Representative flow cytometry plots showing the viability (Hoechst) of thawed WJ-Muse cells and their SSEA-3 marker expression maintenance.

(B) Kaplan-Meier survival analysis (upper panels) and weight loss changes (lower panels) for 30 days of 18 Gy abdominal exposed mice receiving either 50,000 freshly isolated Muse cells (left panels) or 50,000 cryopreserved Muse cells (right panels) 4 hours after irradiation. Statistical difference in survival between groups was determined by Log-rank (Mantel-Cox) test; * p<0.05; ** p≤0.01. Weight data are represented with means±SEM.

FIG. 6. Muse are required for intestinal epithelium regeneration

(A) Intestinal permeability illustrated by histogram plots showing 4-kDa FITC-Dextran levels measured in the plasma of non-treated or Muse-treated mice at 7 days after irradiation, compared to non-irradiated control mice. Data are represented with means±SEM, * p<0.05; **** p<0.001 (two-tailed Mann-Whitney U test).

(B) Representative immunohistochemical images showing the expression of ZO-1 protein (light grey) in ileum sections of non-treated or Muse-treated mice at 7 days after irradiation, compared to non-irradiated control mice. Nuclei were counterstained with hematoxylin (dark grey).

(C) Representative immunofluorescent images showing EpCAM-positive cells (light grey) in the small intestine of non-treated or Muse-treated mice at 7 days after irradiation, compared to non-irradiated control mice. Nuclei were counterstained with DAPI (dark grey).

(D) Representative immunofluorescent images showing CD24-positive cells (white) and Ki67positive cells (light grey) in the small intestine of non-treated or Muse-treated mice at 7 days after irradiation, compared to non-irradiated control mice. Nuclei were counterstained with DAPI (dark grey).

FIG. 7. Monocyte/M2-Macrophage recruitment is induced by Muse treatment

(A) Histogram plots showing MCP-1 secretion level in supernatant of 7 hour-cultured terminal ileum excised from non-treated or Muse-treated at 1 or 7 days after irradiation, compared to non-irradiated control mice. Data are represented with means±SEM, * p<0.05; ** p<0.01 (two-tailed Mann-Whitney U test).

(B) Representative flow cytometry gating strategy for analysis of monocyte subset in lamina propria isolated from small intestine of non-treated (top) or Muse-treated (bottom) mice at 1 day after irradiation. Histograms plots showing the proportion of M1-like monocytes (Ly6Chi) among the alive CD45⁺ population from the lamina propria (left) and lamina epithelialis (right) fractions of non-treated or Muse-treated mice at 1 day after irradiation, compared to nonirradiated control mice (bottom). Data are represented with means±SEM, *p<0.05 (two-tailed Mann-Whitney U test).

(C) Representative immunofluorescence images showing CD68-positive (white) and CD206positive (light grey) macrophages in the small intestine of non-treated or Muse-treated mice at 7 days after irradiation, compared to non-irradiated control mice. Nuclei were counterstained with DAPI (dark grey).

FIG. 8. Muse enhance Paneth cell proliferation and IL-6/Stat3 signalling pathway

(A) Histogram plots showing IL-6 secretion level in supernatant of 7 hour-cultured terminal ileum excised from non-treated or Muse-treated mice at 1 or 7 days after irradiation, compared to non-irradiated control mice. Data are represented with means±SEM, * p<0.05 (two-tailed Mann-Whitney U test).

(B) Representative immunofluorescence images showing Lysozyme-positive cells (light grey) in the small intestine of non-treated or Muse-treated mice at 1 day after irradiation, compared to non-irradiated control mice. Nuclei were counterstained with DAPI (dark greyblue) (top). Histogram plots showing the Paneth cell number per crypts in the 3 groups of mice (bottom). Data are represented with means±SEM, * p<0.05; ** p<0.01 (two-tailed Mann-Whitney U test).

(C) Histograms plots showing the total cell number in the lamina epithelialis fraction (left) and the proportion of Paneth cells (CD24hiCD166med/+) among CD45neg population (right) in nontreated or Muse-treated mice at 1 day after irradiation. Data are represented with means±SEM, ** p<0.01 (two-tailed Mann-Whitney U test).

(D) Representative cropping western blot showing the protein level of cleaved caspase-3 in Paneth cells isolated from two non-treated and two Muse-treated mice at 1 day after irradiation (left). GAPDH was used as an internal control. Full length blots are presented in FIG. 9C. Quantitative analysis of cleaved caspase 3 protein level normalized to GAPDH level (right). Data are represented with means±SEM.

(E) Representative immunofluorescence images showing Lysozyme-positive cells (white) and Ki67-positive cells (light grey) in the small intestine of non-treated or Muse-treated mice at 1 day after irradiation, compared to non-irradiated control mice. Nuclei were counterstained with DAPI (dark grey).

(F) Representative cropping western blot showing the protein levels of p-Stat3 and Stat3 in Paneth cells isolated from two non-treated and two Muse-treated mice at 1 day after irradiation. GAPDH was used as an internal control (left). Full length blots are presented in FIG. 9D. Quantitative analysis of the p-Stat3/Stat3 ratio after normalization to GAPDH level (right). Data are represented with means±SEM. * p<0.05 (two-tailed MannWhitney U test).

FIG. 9.

(A) Representative flow cytometry gating strategy for analyses and isolation of lamina epithelialis subpopulations enriched in stem cells (Epcam⁺CD44⁺Cd24^{low}CD166^{neg/hi} or Paneth cells (Epcam⁺CD44⁺CD24^{hi}C166^{med/pos}).

(B) Quantitative RT-qPCR analysis showing the expression markers of stem cells (Lgr5, Olfm4, Ascl2) and Paneth cells (Lysozyme) in isolated subpopulations, compared to CD24/CD166 double negative intestinal epithelial cells (black box).

### EXAMPLES

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

### Example: Foetal Muse-Based Therapy Prevents Lethal Radio-Induced Gastrointestinal Syndrome by Intestinal Regeneration

### 1. Materials and Methods

1.1. Cell preparation and culture

Human adult bone marrow Mesenchymal Stem cells (BM-MSC) derived from healthy donors were purchased from Lonza (#PT-2501).

Human foetal MSC (WJ-MSC) were derived from Wharton's jelly of umbilical cords (UC), obtained from normal full-term deliveries after maternal informed consent at Maternity unit from Bicêtre

AP-HP Hospital (Le Kremlin-Bicêtre, France). According to the French law, article L. 1243-3 of the Public Health Code, a prior approval by an Institutional Review Board was not required. WJ-MSC

were isolated as previously described [55]. Briefly, UC were collected and placed in a transport solution containing phosphate buffer saline (Gibco™ DPBS, #14190144, Fisher scientific) supplemented with 1 mM EDTA (#E177, VWR), 4% ACD-A (Macopharma) and 0.5% of human serum albumin (hSA, Vialebex, LFB France). Then, UC were incubated for 1h30 in an antibiotic/antifungal bath containing NaCl, 1 g/L Vancomycin (GSK, United Kingdom), 1 g/L Clamoxyl® (GSK, United Kingdom), 0.5 g/L Amikacine (Mylan, France) and 0.05 g/L Fungizone (Bristol Myers Squibb, France). Then, UC were cut in 2 cm-long pieces and frozen in a solution of 50% Gibco™ RPMI-1640 (Life Technologies)+50% glycerol (Sigma-Aldrich) before storage at −80° C. until WJ-MSC isolation.

UC pieces were thawed after a resting period of 30 min at room temperature (RT), cut into smaller pieces around 1-2 mm³ and digested for 1 h at 37° C. in a solution of DPBS containing 3 mM CaCl₂), 300 U/mL collagenase type I (#17100017, Fisher scientific) and 1 mg/mL hyaluronidase (#HX0514, Calbiochem-Merck) and were then placed in a DPBS+0.025% trypsin-EDTA (#R001100, Fisher scientific) for 30 min at 37 °C. After filtration through a 100 μm cell strainer and centrifugation at 200 g for 10 min, cells were seeded at a 12,000 cells/cm² density in a MEMα medium (#01-042-1, Biological Industries) supplemented with 0.01 mg/ml ciprofloxacine (Panpharma, France), 2 U/ml heparin (Choay, France) and 5% platelet lysate (obtained from platelet apheresis collection performed at the “Centre de Transfusion Sanguine des Armées”, Clamart, France). WJ-MSC were amplified until passage 2 and frozen in MEMα supplemented with 10% hSA and 10% dimethylsulfoxide (Sigma-Aldrich) for storage at −80 °C.

For Muse cell isolation, BM- or WJ-MSC were cultured at a 15,000 cells/cm² density in a Gibco™ Low-glucose DMEM+GlutaMAX medium (#11570586, Fisher scientific) with 10% foetal bovine serum (FBS, #SH30071.03IH, HyClone™, Fisher scientific), 1 ng/ml human FGF-2 (#130-093-564, Miltenyi Biotec) and 0.1 mg/ml Gibco™ kanamycin sulphate (#11578876, Fisher scientific) at 37° C. in 95% air and 5% CO₂. Cells from passage 7 were used for multilineage-differentiating stress-enduring (Muse) cell isolation. GFP-labelled WJ-MSC were generated using lentiviral plasmid pTrip-MND-GFP.

### 1.2. Mass Spectrometry-Based Proteomic Analyses

Three biological replicates of WJ-Muse and BM-Muse cells were analysed. The proteins were directly solubilized in Laemmli buffer, heated for 10 min at 95° C. and stacked in the top of a 412% NuPAGE gel (Invitrogen). After staining with R-250 Coomassie Blue (Biorad), proteins were digested

in-gel using trypsin (modified, sequencing purity, Promega), as previously described [56]. The resulting peptides were fractionated by tip-based strong cation exchange (3M Empore). For this, peptides were dissolved in 5% acetonitrile (ACN), 1% trifluoroacetic acid and eluted in 4 fractions (F1: 100 mM ammonium acetate (AA), 20% ACN, 0.5% formic acid (FA); F2: 175 mM AA, 20% ACN, 0.5% FA; F3: 375 mM AA, 20% ACN, 0.5% FA; F4: 80% ACN, 5% ammonium hydroxide) before desalting using C18 reverse phase chromatography (Ultra-Micro SpinColumns, Harvard Apparatus). NanoLC-MS/MS analyses of peptides eluted in each fraction were performed using an Ultimate 3000 RSLCnano coupled to a Q-Exactive HF (Thermo Fisher Scientific) using a 240-min gradient. For this purpose, the peptides were sampled on a precolumn (300 μm×5 mm PepMap C18, Thermo Scientific) and separated in a 75 μm×250 mm C18 column (Reprosil-Pur 120 C18-AQ, 1.9 μm, Dr. Maisch). The MS and MS/MS data were acquired by Xcalibur (Thermo Fisher Scientific). Peptides and proteins were identified by Mascot (version 2.6.0, Matrix Science) through concomitant searches against the Uniprot database (Homo sapiens taxonomy, October 2019 version), a homemade database containing the sequences of classical contaminant proteins found in proteomic analyses (bovine albumin, keratins, trypsin, etc.), and the corresponding reversed databases. Trypsin/P was chosen as the enzyme and two missed cleavages were allowed. Precursor and fragment mass error tolerances were set at respectively at 10 ppm and 25 mmu. Peptide modifications allowed during the search were: Carbamidomethyl (C, fixed), Acetyl (Protein N-term, variable) and Oxidation (M, variable). The Proline software was used for the compilation, grouping, and filtering of the results (conservation of rank 1 peptides, peptide length ≥7 amino acids, peptide-spectrum-match score ≥25, false discovery rate of peptide-spectrum-match identifications <1% as calculated on peptide-spectrum-match scores by employing the reverse database strategy, and minimum of one specific peptide per identified protein group). Proline was then used to perform MS1 label-free quantification of the identified protein groups. Statistical analysis was then performed using the ProStaR software [59]. Proteins identified in the contaminant database and proteins detected in less than three replicates of one condition were removed. After log 2 transformation, abundance values were normalized by median centering, before missing value imputation (slsa algorithm for partially observed values in the condition and DetQuantile algorithm for totally absent values in the condition). Statistical testing was then conducted using limma, whereby differentially expressed proteins were sorted out using a fold change cut-off of 1 and a p-value cut-off of 0.005, leading to an FDR <1% according to the Benjamini-Hochberg estimator. Proteins found differentially abundant, but detected in less than three replicates in the condition in which they were found to be more abundant, were manually invalidated (p-value=1). Gene Ontology (GO) term enrichment analysis was performed with DAVID Bioinformatics resources.

### 1.3. SSEA-3 Staining for Muse Cell Isolation

When overconfluent, cells were washed, trypsinized and centrifuged for 5 min at 400g. Pellet was resuspended in FluoroBrite™ DMEM (#A1896701, Fisher scientific) for washing and then centrifuged. After discarding the supernatant, cells were resuspended in a freshly prepared cold FACS buffer (FluoroBrite™ DMEM containing 0.5% bovine serum albumin (BSA) and 2 mM EDTA) at a maximal concentration of 1×10⁶ cells/100 μl. Cells were incubated on ice for 1 h with anti-SSEA-3 antibody at a 1:250 dilution (mixing gently every 10 min) (#MAB4303-1, Millipore). Cells were then centrifuged and washed 3 times with FACS buffer. A secondary anti-rat IgM antibody conjugated to APC (#112-136-075, Jackson ImmunoResearch Lab.) was added to the cells for 1 h on ice at a 1:100 dilution (mixing gently every 10 min). Cells were centrifuged and washed 3 times with FACS buffer. After filtration through a 30 μm cell strainer, SSEA-3⁺ cells were sorted with a BD FACS Aria II SORP cell sorter (Becton Dickinson) using a 100 μm nozzle. Freshly sorted SSEA-3⁺ (Muse) cells were washed with DPBS, centrifuged and resuspended in sterile DPBS (50,000 cells in 100 μl/mouse) for intravenous (i.v.) injection.

GFP-labelled WJ-Muse were isolated as SSEA-3⁺ cells from GFP⁺WJ-MSC.

### 1.4. Bulk Production of Muse Cell Derived-Clusters by Using Methylcellulose Gel

To avoid cell adherence, plates were pre-coated with a 3% poly-HEMA solution in 95% Ethanol [poly(2-hydroxyethyl methacrylate), #P3932, Sigma-Aldrich]. FACS-sorted Muse cells were mixed to Low-glucose DMEM+GlutaMAX medium containing 10% FBS and 0.9% methylcellulose (MethoCult H4100, #04100, StemCell Technologies). After plating, cells were maintained in culture for 7 to 10 days by addition of fresh Low-glucose DMEM+GlutaMAX medium containing 10% FBS every 3 days.

Then, clusters were picked up for analysis.

### 1.5. Muse Cell Cryopreservation

FACS isolated SSEA-3⁺Muse cells were centrifuged (300 g, 5 min) immediately after sorting. Supernatant was discarded and cells were resuspended in 1 ml BAMBANKER™ (#W1W30214681, Sobioda) and transferred into a cryotube. After a 24 h freezing at −80° C. (isopropanol freezing container), tubes were stored into liquid nitrogen.

The day of injection, cells were thawed in a water bath at 37° C. and added to 9 ml of prewarmed FBS. After centrifugation, pellet was resuspended in sterile DPBS (50,000 cells in 100 μl/mouse) for i.v. injection.

### 1.6. Evaluation of Pluripotency and of Differentiation Ability of Muse Cells by Quantitative RT-PCR

Total RNA was extracted with the RNeasy Plus Micro kit (#74034, QIAGEN) and reverse transcribed with random primers and Superscript IV (#18090050, Invitrogen). Quantitative PCR reactions were performed using the Power SYBR™ green Master mix (#4367659, ThermoFisher Scientific) in a StepOne™ Real-Time PCR System (Applied Biosystems).

Primer sequences are listed in Table 1 below.

TABLE 1
List of human (h) and murine (m)
qRT-PCR primers

| | Forward primers | | Reverse primers | |
|---|---|---|---|---|
| | SEQ | | SEQ | |
| | ID | | ID | |
| Genes | NO | Sequence | NO | Sequence |
| h-Beta- | SEQ | 5′-CACCATT | SEQ | 5′-AGGTCTT |
| actin | ID | GGCAATGAGC | ID | TGCGGATGTC |
| | NO: | GGTTC | NO: | CACGT |
| | 1 | | 2 | |
| h-Sox2 | SEQ | 5′-GTCACAG | SEQ | 5′-TCTGCGA |
| | ID | CATGATGCAG | ID | GCTGGTCATG |
| | NO: | GACCA | NO: | GAGTT |
| | 3 | | 4 | |
| h-Nanog | SEQ | 5′-CTCCAAC | SEQ | 5′-CGTCACA |
| | ID | ATCCTGAACC | ID | CCATTGCTAT |
| | NO: | TCAGC | NO: | TCTTCG |
| | 5 | | 6 | |
| h-Oct3/4 | SEQ | 5′-CCTGAAG | SEQ | 5′-AAAGCGG |
| | ID | CAGAAGAGGA | ID | CAGATGGTCG |
| | NO: | TCACC | NO: | TTTGG |
| | 7 | | 8 | |
| h-CK18 | SEQ | 5′-CAAGGAG | SEQ | 5′-TGGTGCT |
| | ID | GAGCTGCTCT | ID | CTCCTCAATC |
| | NO: | TCATG | NO: | TGCTG |
| | 9 | | 10 | |
| h-OCLN | SEQ | 5′-CCTATAA | SEQ | 5′-AACGAGG |
| | ID | ATCCACGCCG | ID | CTGCCTGAAG |
| | NO: | GTTC | NO: | TCA |
| | 11 | | 12 | |
| h-IDO | SEQ | 5′-TCTCATT | SEQ | 5′-GTGTCCC |
| | ID | TCGTGATGGA | ID | GTTCTTGCAT |
| | NO: | GACTGC | NO: | TTGC |
| | 13 | | 14 | |
| h-TGFbeta1 | SEQ | 5′-GGCCAGA | SEQ | 5′-GTGGGTT |
| | ID | TCCTGTCCAA | ID | TCCACCATTA |
| | NO: | GC | NO: | GCAC |
| | 15 | | 16 | |
| h-Cox2 | SEQ | 5′-CAGCCAT | SEQ | 5′-ATCCTGT |
| | ID | ACAGCAAATC | ID | CCGGGTACAA |
| | NO: | C | NO: | T |
| | 17 | | 18 | |
| h-PD-L1 | SEQ | 5′-GGACAAG | SEQ | 5′-CCCAGAA |
| | ID | CAGTGACCAT | ID | TTACCAAGTG |
| | NO: | CAAG | NO: | AGTCCT |
| | 19 | | 20 | |
| m-GAPDH | SEQ | 5′-AAGGTGA | SEQ | 5′-GGGGTCA |
| | ID | AGGTCGGAGT | ID | TTGATGGCAA |
| | NO: | CAAC | NO: | CAATA |
| | 21 | | 22 | |
| m-Nos2 | SEQ | 5′-GGTCTTT | SEQ | 5′-CACAACT |
| | ID | GACGCTCGGA | ID | GGGTGAACTC |
| | NO: | ACTGTAG | NO: | CAAGGTG |
| | 23 | | 24 | |
| m-Arg1 | SEQ | 5′-GTGAAGA | SEQ | 5′-CTGGTTG |
| | ID | ACCCACGGTC | ID | TCAGGGGAGT |
| | NO: | TGT | NO: | GTT |
| | 25 | | 26 | |
| m-Lgr5 | SEQ | 5′-CCTACTC | SEQ | 5′-GCATTGG |
| | ID | GAAGACTTAC | ID | GGTGAATGAT |
| | NO: | CCAGT | NO: | AGCA |
| | 27 | | 28 | |
| m-Lysozyme | SEQ | 5′-TGACATC | SEQ | 5′-TGGGACA |
| | ID | ACTGCAGCCA | ID | GATCTCGGTT |
| | NO: | TAC | NO: | TTG |
| | 29 | | 30 | |
| m-Olfm4 | SEQ | 5′-CAGCCAC | SEQ | 5′-GCTGGAC |
| | ID | TTTCCAATTT | ID | ATACTCCTTC |
| | NO: | CACTG | NO: | ACCTTA |
| | 31 | | 32 | |
| m-Ascl2 | SEQ | 5′-AGGTCAG | SEQ | 5′-TCCTGGT |
| | ID | TCAGCACTTG | ID | GGACCTACCT |
| | NO: | GCATT | NO: | GCTT |
| | 33 | | 34 | |
| m-Hprt | SEQ | 5′-GCTGGTG | SEQ | 5′-CACAGGA |
| | ID | AAAAGGACCT | ID | CTAGAACACC |
| | NO: | CT | NO: | TGC |
| | 35 | | 36 | |

### 1.7. Evaluation of Immune Privilege of Muse Cells

10⁴ isolated human Muse cells were co-cultured with, either 10⁵ Ficoll-isolated human peripheral blood mononuclear cells (hPBMC), or 10⁵ isolated murine splenic lymphocytes (mSL) in Gibco™ RPMI-1640⁺GlutaMAX medium (#72400-021, Fisher scientific) containing 1 mM sodium pyruvate (#11360-070, Gibco), 1× MEM Non-Essential Amino Acids solution (NEAA, #11140-035, Gibco), 5 UM 2-mercaptoethanol (#31350010, Gibco), 10% FBS and 100 U/ml Gibco™ penicillin streptomycin (#15140122, Fisher scientific). Six days later, 10 μM 5bromo-2′-deoxyuridine (BrdU) was added to the medium for 2 hours and cells were collected to measure BrdU incorporation in CD3⁺ lymphocytes by flow cytometry. As positive controls of proliferation, hPBMC or mSL were treated with 5 UM of concanavalin A (#C5275, SigmaAldrich). Briefly, cells were stained with anti-human or anti-mouse CD3 antibody for 15 min at 4 °C. After washing, cells were fixed and permeabilized with the BD Cytofix/Cytoperm™ kit (#554722, BD Biosciences) following the manufacturer's protocol. After a DNAse I treatment for 1 h at 37° C., cells were incubated with a FITC anti-BrdU antibody for 30 min at RT. After washing and centrifugation, cells were resuspended in DPBS and analysed by FACS.

### 1.8. Evaluation of the Immunosuppressive Potential (Allogeneic and Xenogeneic) of Muse Cells

5,000 or 10,000 or 20,000 isolated human Muse cells were cultured in 96-well plates at 37° C., in presence or not of 15 ng/ml TNFα and 10 ng/ml IFNγ. After 48h, Muse cells were washed with DPBS before co-culturing them with 100,000 hPBMC or mSL in Gibco™ RPMI-1640⁺GlutaMAX medium containing 1 mM sodium pyruvate, 1× NEAA, 5 μM 2-mercaptoethanol, 10% FBS and 100 U/ml Gibco™ penicillin streptomycin for a 2 hour-contact. Then, 5 μM concanavalin A was added to the medium, and after 6 days of culture, cells were incubated for 2 hours with 10 UM BrdU and collected to measure BrdU incorporation in CD3⁺T-lymphocytes by flow cytometry.

1.9. In vitro Differentiation of Muse cells

Multipotency of Muse cell was assessed by testing their ability to differentiate into adipocytes, osteoblasts and epithelial cells in presence of specific differentiation media.

Isolated cells were cultured overnight at a 15,000 cells/cm² density in a Gibco™ Low-glucose DMEM+GlutaMAX medium with 10% FBS, 1 ng/ml human FGF-2 and 0.1 mg/ml Gibco™ kanamycin sulfate, at 37° C. in 95% air and 5% CO₂.

Epithelial differentiation: 10 μM retinoic acid (#R2625, Sigma-aldrich) was added to the medium, which was replaced every 2-3 days. After 4 weeks, cells were washed with DPBS and placed in RLT buffer to extract RNA for qRT-PCR.

Osteogenic differentiation: DMEM medium was replaced by a StemXVivo Osteogenic/Adipogenic Base Media (#CCM007, R&D Systems) with 1% kanamycin sulfate and 5% StemXVivo human osteogenic supplement 20× (#CCM008, R&D Systems), which was replaced every 2-3 days. After 2 weeks, cells were washed with DPBS and fixed with 4% paraformaldehyde for immunological (osteocalcin) staining.

Adipogenic differentiation: DMEM medium was replaced by a StemXVivo Osteogenic/Adipogenic Base Media (#CCM007, R&D Systems) with 1% kanamycin sulfate and 1% StemXVivo human adipogenic supplement 100× (#CCM0011, R&D Systems), which was replaced every 2-3 days. After 3 weeks, cells were washed with DPBS and fixed with 4% paraformaldehyde for histological (Oil red O) staining.

### 1.10. Co-culturing of BMDM and WJ-MuselMSC

BMDM were isolated using standard protocols [60]. Primary macrophages were derived from murine bone marrow cells and were cultured alone or in presence of 50,000 WJ-Muse or 50,000 MSC (ratio 1:1) in IMDM supplemented with 10% FBS, 1% penicillin streptomycin, 10 mM 1-thioglycerol (#M1753, Sigma-Aldrich) and 25 ng/ml mouse M-CSF (#130-101-706, Miltenyi Biotec). After 7 days of culture, BMDM were collected in RLT buffer for RNA extraction.

### 1.11. Mice

Eight-week-old C57BL/6JRj male mice were purchased from Janvier Laboratory (Le Genest Saint Isle, France) and were housed in the IRSN specific-pathogen-free-animal facility, accredited by the French Ministry of Agriculture to perform experiments on rodents.

Experimental procedures were performed in accordance with French and European regulations on the protection of animals used for scientific purposes (EC Directive 2010/63/EU and French Decree 2013-118). All experiments were approved by the Ethics Committee #81 and authorized by the French Ministry of Research (under the reference APAFIS #174922018111211126943-v1). In this study, experimental protocol was in line with standard support treatment recommended for patient presenting an acute radiation syndrome [61]. Thus, mice received antibiotics in drinking water (8 g/L Avemix®) during the whole study.

### 1.12. Irradiation and Treatment

Mice were irradiated under continuous anaesthesia (1.5% isoflurane in oxygen) with a medical linear accelerator (Elekta Synergy) delivering 4MVp X-rays. Reference dosimetry measurements were performed using a 0.125 cm3 cylindrical ionization chamber, calibrated in dose to water in a mouse equivalent tissue phantom placed on a Plexiglas support. A localized 2 cm-large abdominal irradiation window containing intestine was determined to avoid an exposure of the upper thorax and of extremities. Mice were irradiated at 18 Gy with a 2.5 Gy/min dose-rate.

### 1.13. Histology and Immunostaining

At days 1, 3.5, 7 and 30 post-irradiation, freshly isolated ileal tissue was excised, flushed with cold

DPBS and a piece of 1 cm-long was fixed in 4% paraformaldehyde before embedding in paraffin., 5 μm-thick sections were de-paraffinized, rehydrated and stained with a hematoxylin/eosin/safran (HES) coloration.

For immunofluorescent staining, a pretreatment method using antigen retrieval with pH 6 citrate buffer (#ZUC028-500, Zytomed systems) was used. Sections were then permeabilized for 10 min at RT with 0.1% Triton X-100 in DPBS containing calcium and magnesium (DPBS Ca⁺⁺Mg⁺⁺#14040091, Fisher scientific) and the non-specific binding was blocked for 30 min with a solution of 5% normal goat serum (NGS) and 1% BSA in DPBS Ca⁺⁺Mg⁺⁺. Sections were then incubated overnight at 4° C. with primary antibodies listed in Table 2 below. After washing, sections were probed with appropriate fluorescent-conjugated secondary antibodies for 45 min at RT and cell nuclei were stained for 5 min with 1 μg/ml DAPI.

ZO-1 immunohistochemical staining was performed on a VENTANA BenchMark Ultra automated staining instrument (Ventana Medical Systems) according to the manufacturer's protocol [62]. Slides were counterstained with hematoxylin.

TABLE 2
List of antibodies used in immunohistology

| Antigen | Clone | Reference | Supplier |
|---|---|---|---|
| Osteocalcin | 190125 | MAB1419 | R&D Systems |
| Lysozyme | EPR2994 | Ab108508 | Abcam |
| Ki67 | — | Ab15580 | Abcam |
| CD24 | M1/69 | Ab64064 | Abcam |
| CD68 | — | Ab125212 | Abcam |
| CD206 | — | AF2535 | R&D Systems |
| GFP | — | Ab6662 | Abcam |
| ZO-1 | — | 40-2200 | Thermo Fisher scientific |

### 1.14. In Situ Apoptosis Detection

Apoptosis was detected by using the TUNEL assay (in situ Cell Death Detection Kit, Fluorescein, #11684795910, Roche Diagnostics, France) following the manufacturer's protocol. Briefly, after deparaffinization and rehydration, intestinal tissue sections were pretreated with Proteinase K and incubated for 1 h at 37° C. with a mixture of Terminal deoxynucleotidyl Transferase (TdT) enzyme and fluorescein-labelled nucleotide polymers in a cacodylate buffer. Sections were then washed with a solution of 0.05% Tween-20 in DPBS and mounted with a Vectashield medium containing DAPI (#H-1200, Vector).

### 1.15. Intestinal Permeability

In vivo intestinal permeability was measured in mice at day 7 after irradiation, by administrating FITC-Dextran (#46944, Sigma-Aldrich) by gavage 4 h before euthanasia (0.6 mg/g body weight). Blood was harvested by cardiac puncture. Standard curves were obtained by diluting the FITC-dextran in DPBS. The concentration of FITC-Dextran in plasma of differently treated groups of mice was measured with a microplate Luminometer (Mithras LB940, Berthold) at a 485 nm excitation and a 520 nm emission.

### 1.16. Cytokine Quantification in Intestine Supernatant

A piece of 600-700 μg of terminal ileum was placed in a Gibco™ RPMI-1640⁺GlutaMAX medium (#61870036, Fisher scientific) containing 100 U/ml Gibco™ penicillin streptomycin (#15140122, Fisher scientific) and incubated for 7 hours at 37° C. in 95% air and 5% CO₂. Supernatant was then collected, aliquoted after addition of protease inhibitors and stored at 80° C. before use. Levels of inflammatory cytokines were measured with the BD Cytometric Bead Array (CBA) Mouse Inflammation Kit (#552364, BD Biosciences), following the manufacturer's protocol. A FACS Canto II flow cytometer was used for sample acquisition.

1.17. Isolation of Paneth Cells from Intestinal Lamina Epithelialis (LE)

To isolate Paneth cells from intestinal LE, the terminal ileum of mice was excised and washed with cold DPBS. After measurement of length and weight, gut was opened longitudinally, cut into 0.5 cm pieces and incubated for 20 min at 37° C. under agitation (25 rpm) in a pre-digestion buffer containing 10 mM Hepes, 5 mM EDTA, 5% FBS and 1 mM DTT in Hank's Balanced Salt Solution (HBSS) without Ca⁺⁺Mg⁺⁺(#14190, Fisher scientific). After 10 sec vortexing, cell solution was filtered (100 μm) and stored. Tissue pieces were a second time incubated for 20 min at 37° C. under agitation in a fresh pre-digestion buffer. After 10 sec vortexing, cell solution was filtered and mixed to the previous stored cell suspension. After centrifugation, cells were counted (Kova slide) and incubated with FACS antibodies listed in Table 3 below and a viability marker. Cell isolation was performed with a BD FACSAria™ II cell sorter (BD Biosciences).

Paneth cells were defined as: CD45^{neg}, EpCAM⁺, CD44⁺, CD24⁺, CD166^{low} [63].

TABLE 3
List of antibodies used in flow cytometry

| Antigen | Conjugate | Clone | Reference | Supplier |
|---|---|---|---|---|
| SSEA-3 | — | MC-631 | MAB4303-I | Merck Millipore |
| BrdU | FITC | B44 | 347583 | BD Biosciences |
| Human CD3 | PercP-Cy5.5 | SK7 | 981008 | BioLegend |
| Mouse CD3 | PerCP | 145-2C11 | 100328 | BioLegend |
| CD45 | PE/Cy7 | 30-F11 | 103114 | BioLegend |
| CD44 | FITC | IM7 | 103005 | BioLegend |
| CD90 | Brilliant Violet 421 ™ | 5E10 | 328122 | BioLegend |
| CD73 | PercP-Cy5.5 | TY/11.8 | 127214 | BioLegend |
| HLA-DR | PercP-Cy5.5 | Tü39 | 361710 | BioLegend |
| CD105 | PE | MJ7/18 | 12-1051-82 | eBioscience |
| EpCAM (CD326) | APC | G8.8 | 118214 | BioLegend |
| CD166 | PE | eBioALC48 | 12-1661-82 | eBioscience |
| CD24 | Brilliant Violet 421 ™ | M1/69 | 101826 | BioLegend |
| CD11b | PE | M1/70 | 553311 | BD Biosciences |
| Ly6C | APC | HK1.4 | 17593282 | BioLegend |
| Ly6G | PerCP | 1A8 | 127616 | BioLegend |

### 1.18. Capillary Electrophoresis Immunoassay (Simple Western)

Total proteins from intestinal lamina epithelialis or from isolated Paneth cells were extracted with a RIPA buffer containing a cocktail of 1× protease inhibitors (#11836145001, Roche) and 1× phosphatase inhibitors (#P2850; #P5726, Sigma-Aldrich).

After 5 min denaturation at 95° C. in the presence of 5× Fluorescent Master Mix (PS-FL01-8, ProteinSimple), samples were assayed on a ProteinSimple Wes automated capillary-based electrophoresis instrument with Wes Separation Module protocol (ProteinSimple). The 12-230 kDa Separation Module 8×25 capillary cartridges (SM-W004, ProteinSimple) and the AntiRabbit Detection Module (DM-001, ProteinSimple) were used. Proteins were identified using rabbit primary antibodies listed in Table 4 below. Results (peak area) were analyzed using Compass for SW software v5.0.1.

TABLE 4
List of antibodies used in Simple Western

| Antigen | Clone | Reference | Supplier |
|---|---|---|---|
| Phospho-Stat3 (Tyr705) | D3A7 | 9145 | Cell Signaling Technology |
| Stat3 | 79D7 | 4904 | Cell Signaling Technology |
| Cleaved caspase-3 | 5A1E | 9664 | Cell Signaling Technology |
| Lysozyme | EPR2994 | Ab108508 | Abcam |
| GAPDH | 14C10 | 2118 | Cell Signaling Technology |

### 2. Results

2.1. Comparative characterization of human Muse cells (Muse) purified from Warton's jelly and Bone Marrow

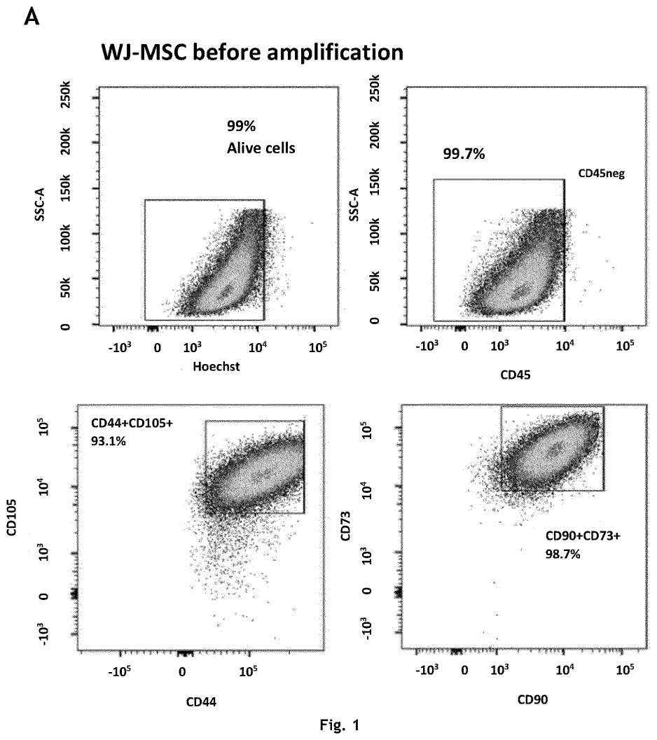

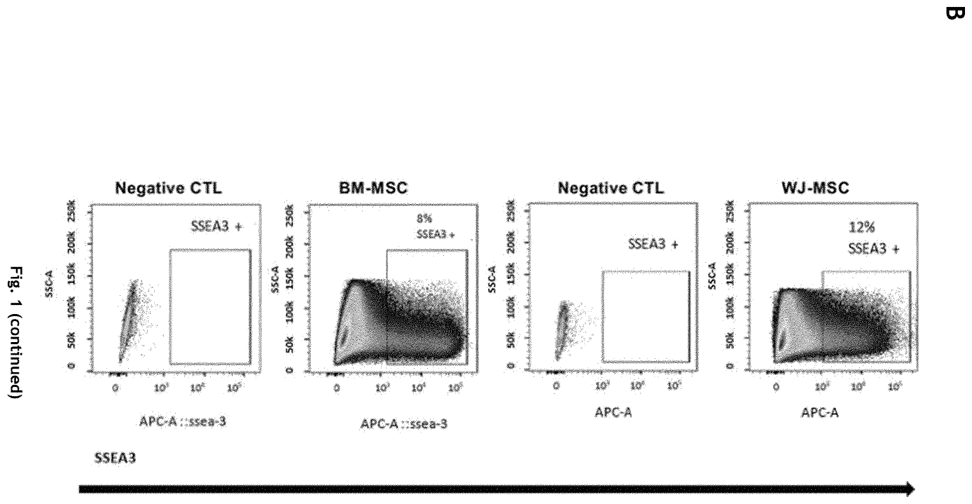

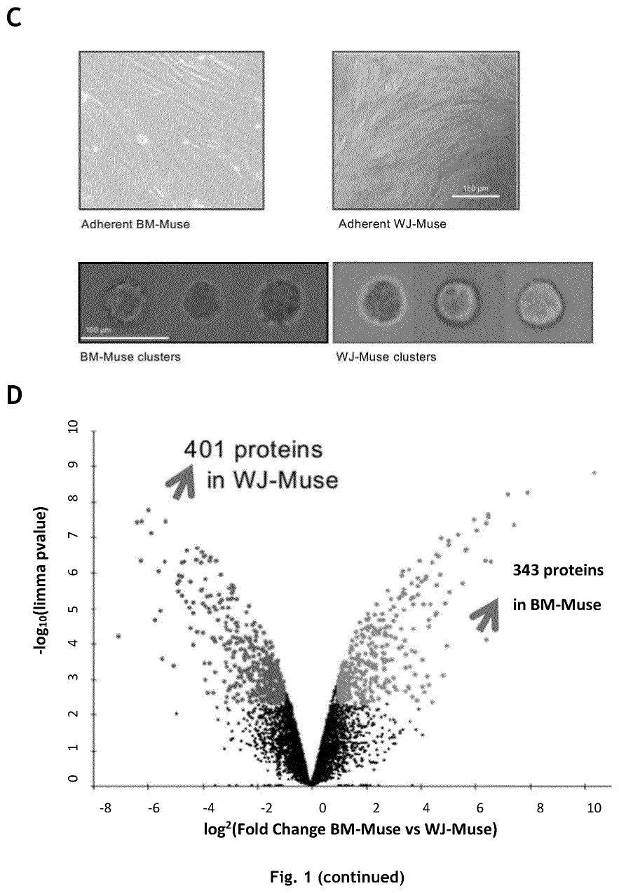

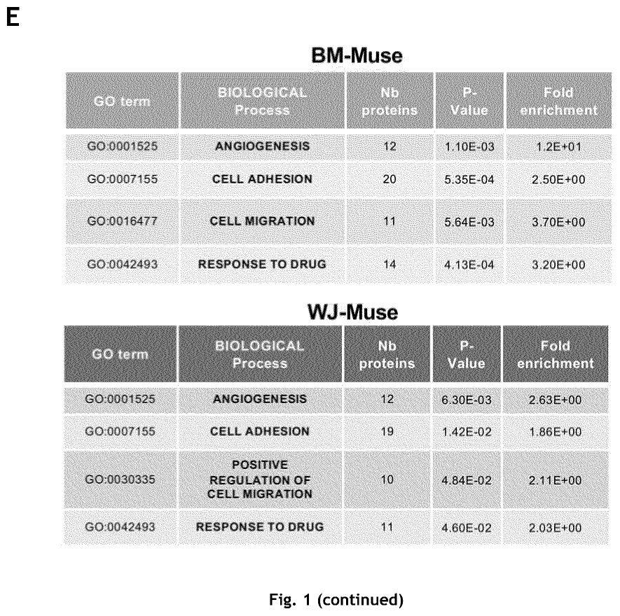

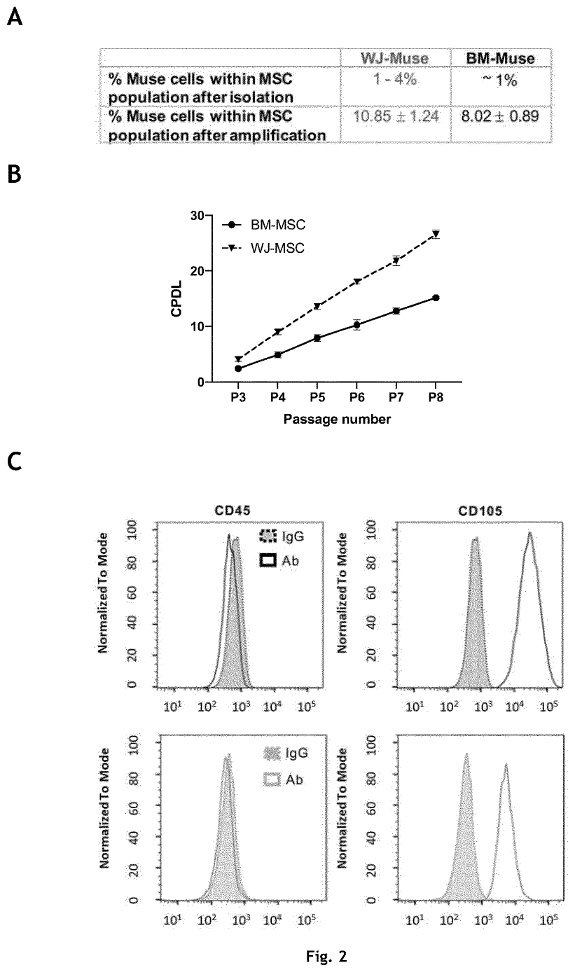

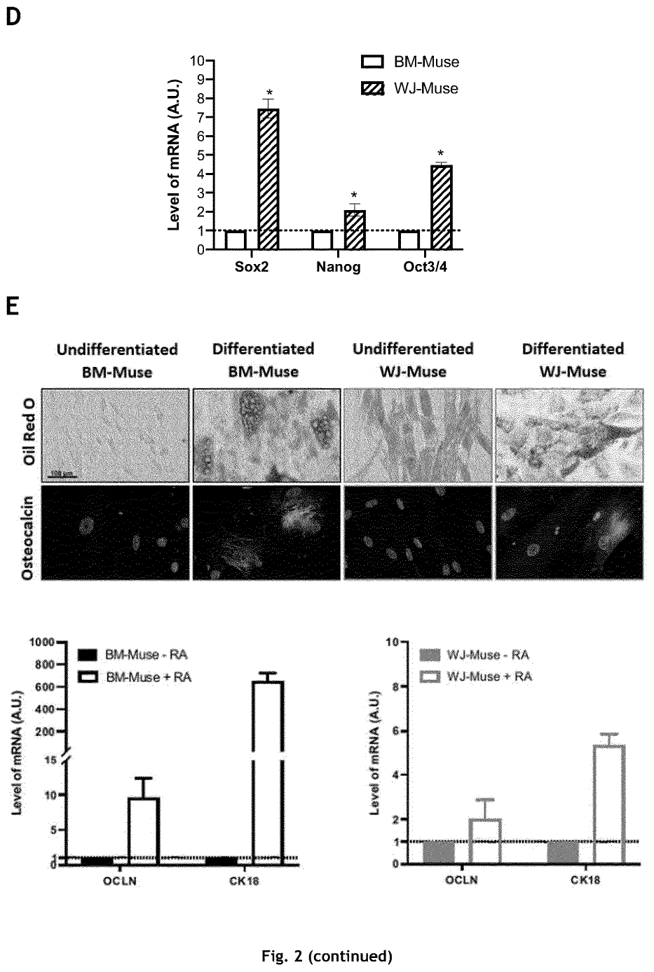

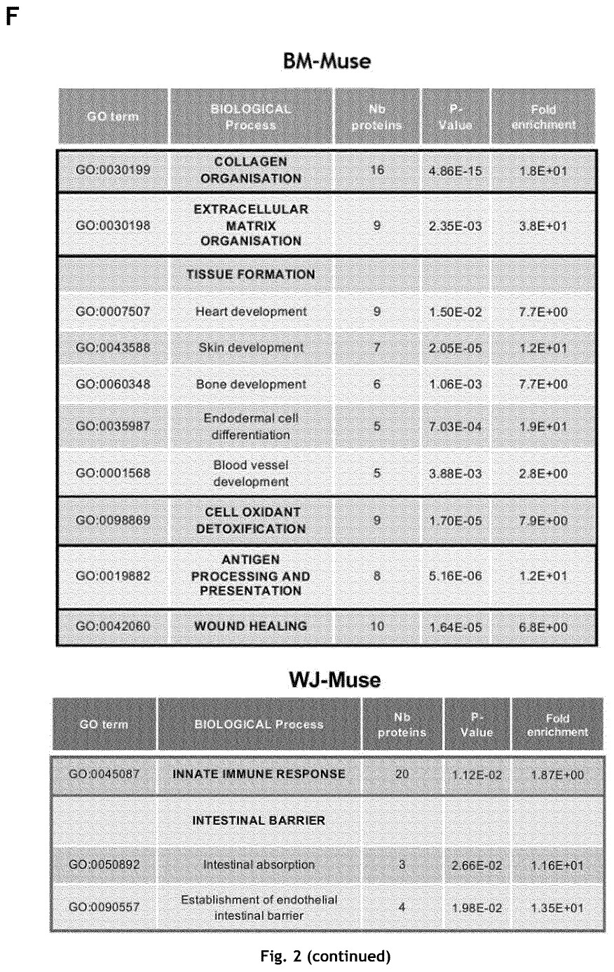

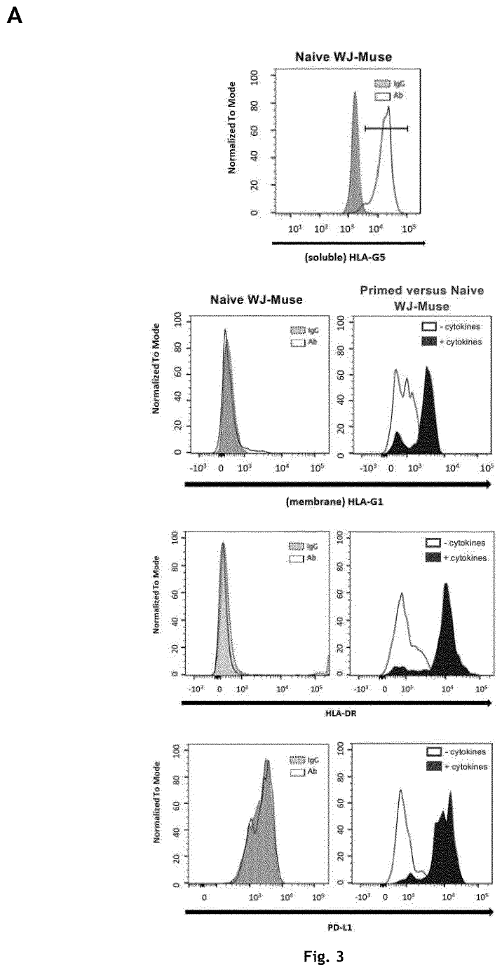

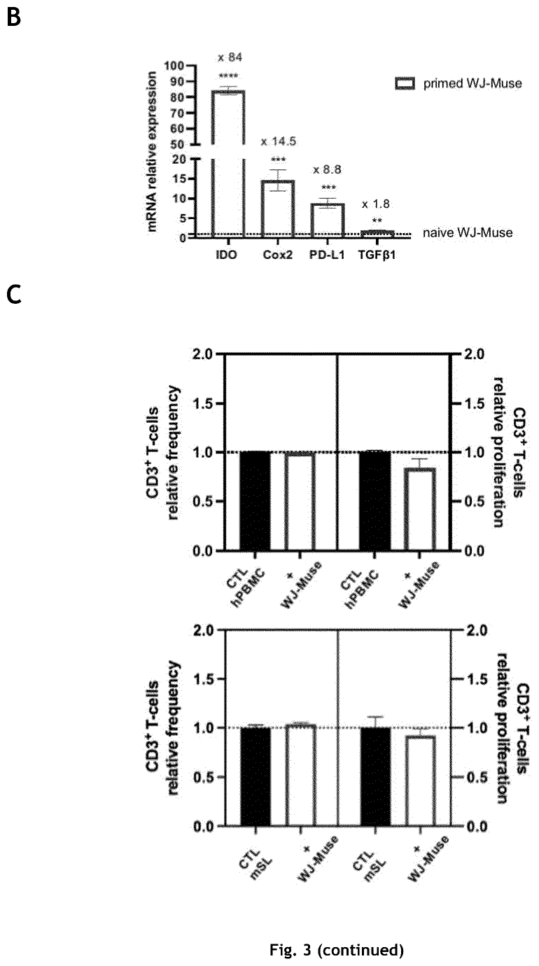

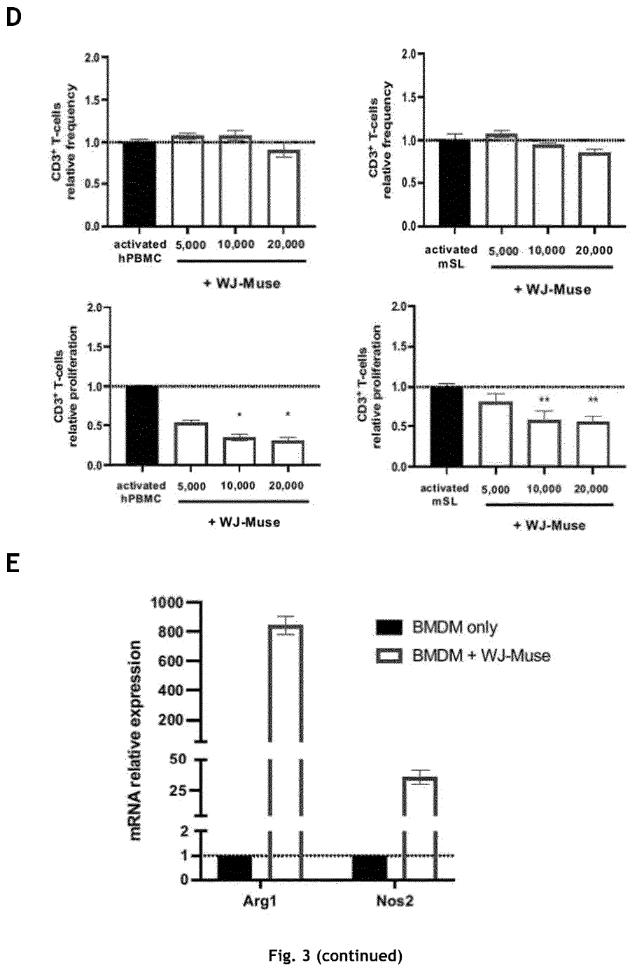

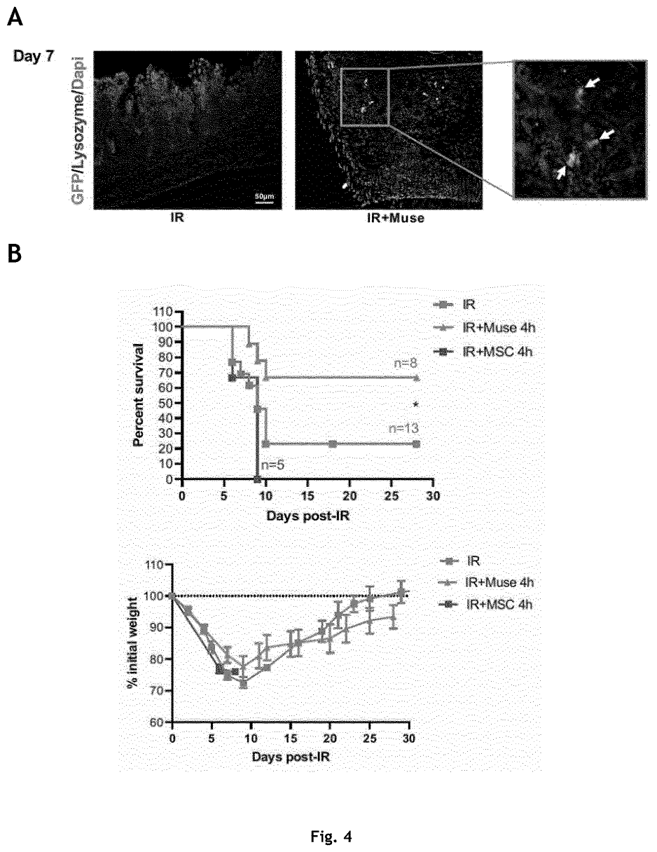

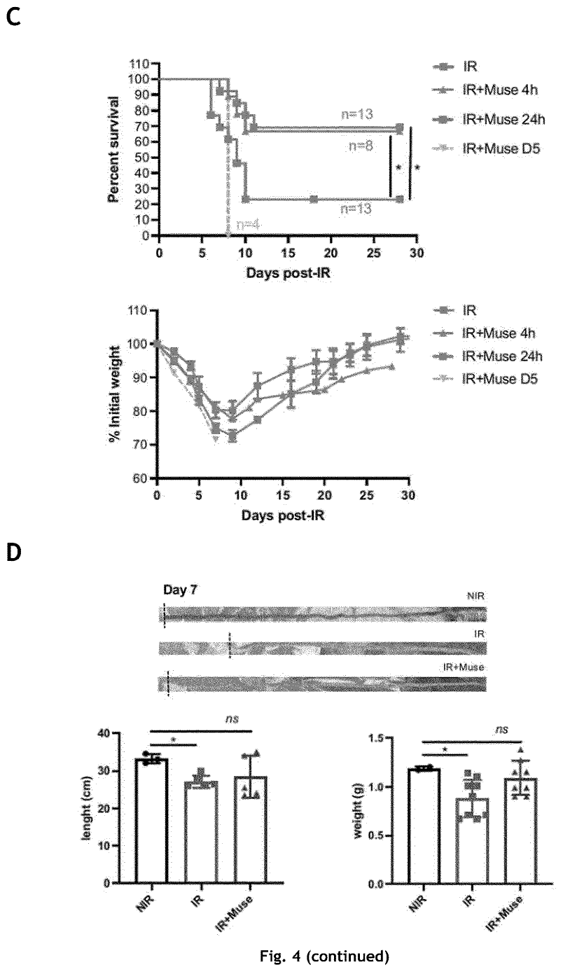

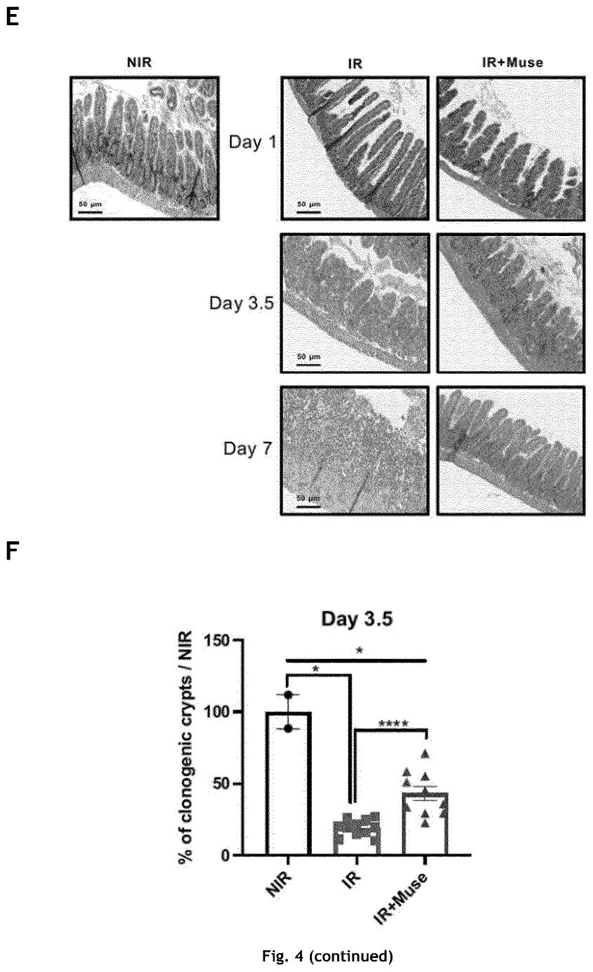

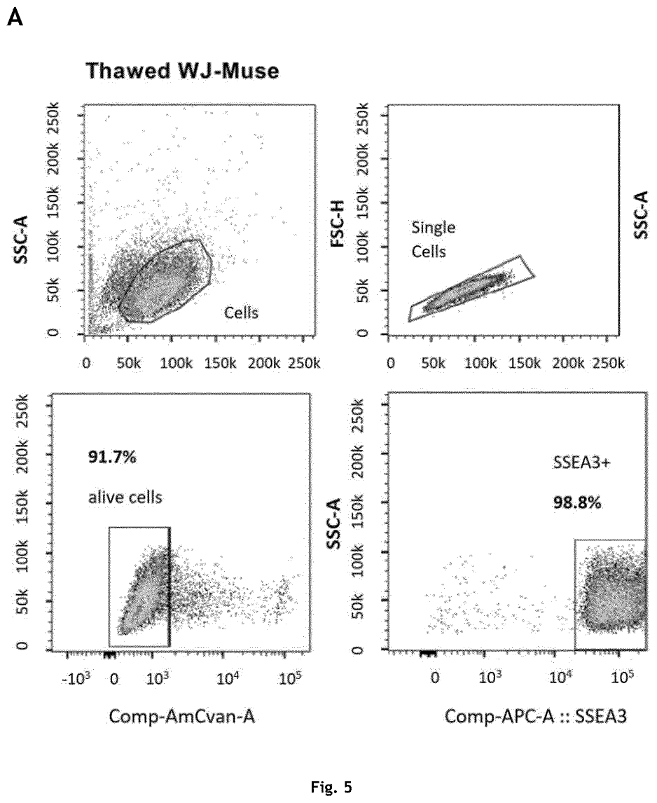

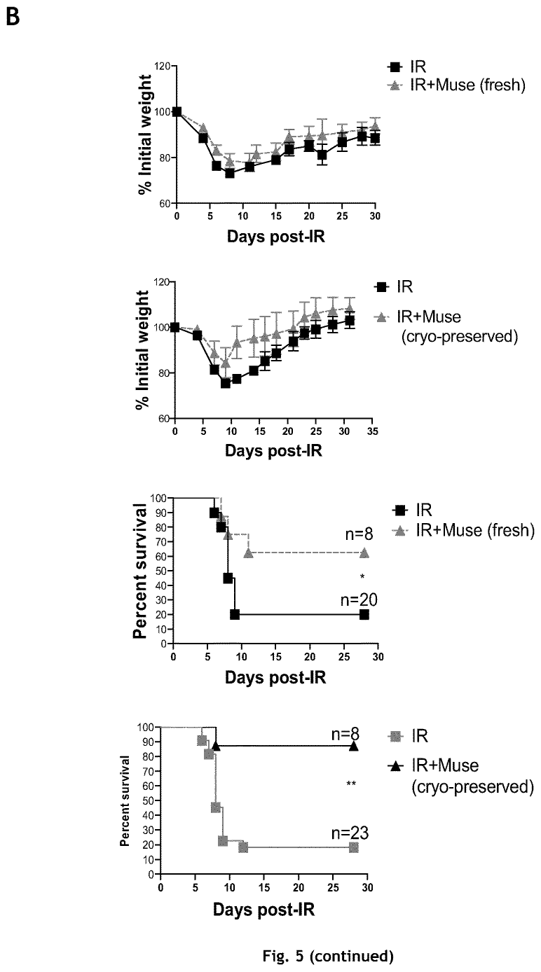

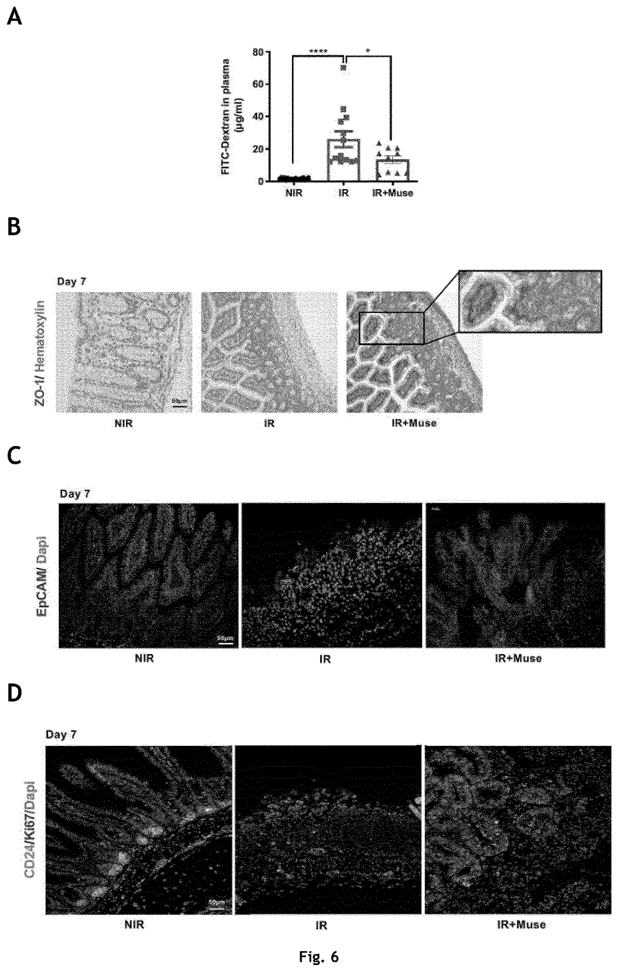

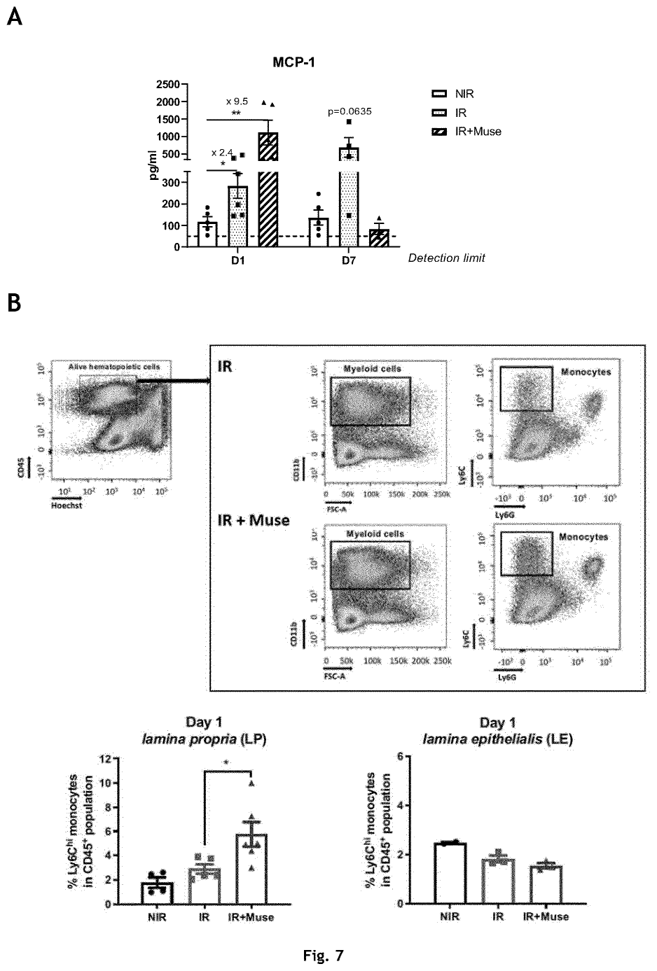

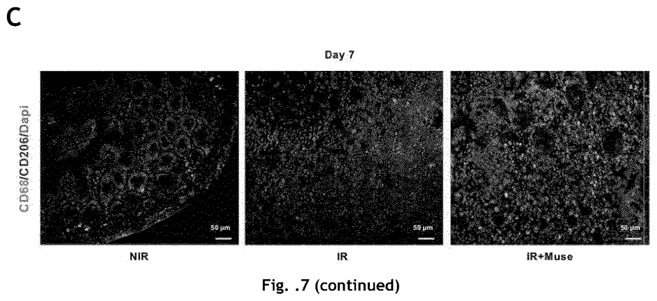

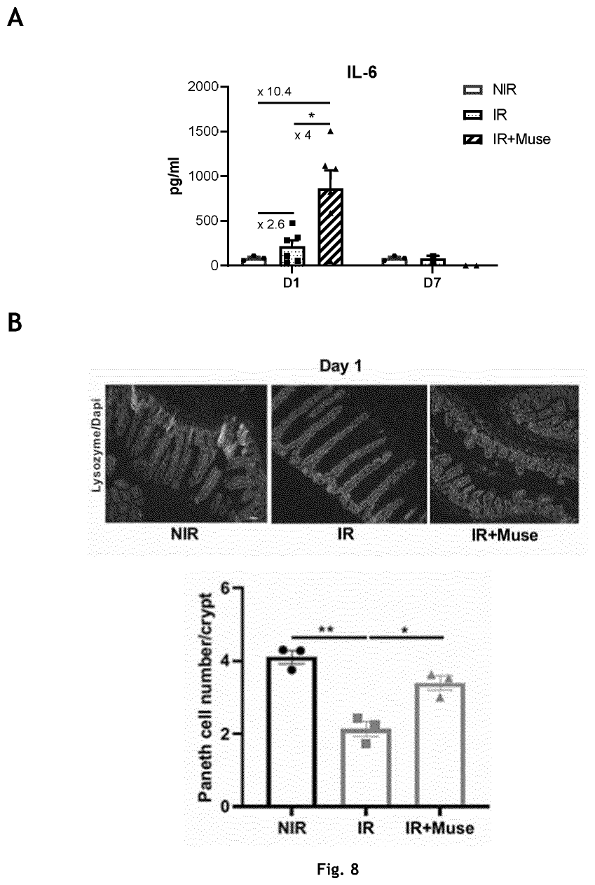

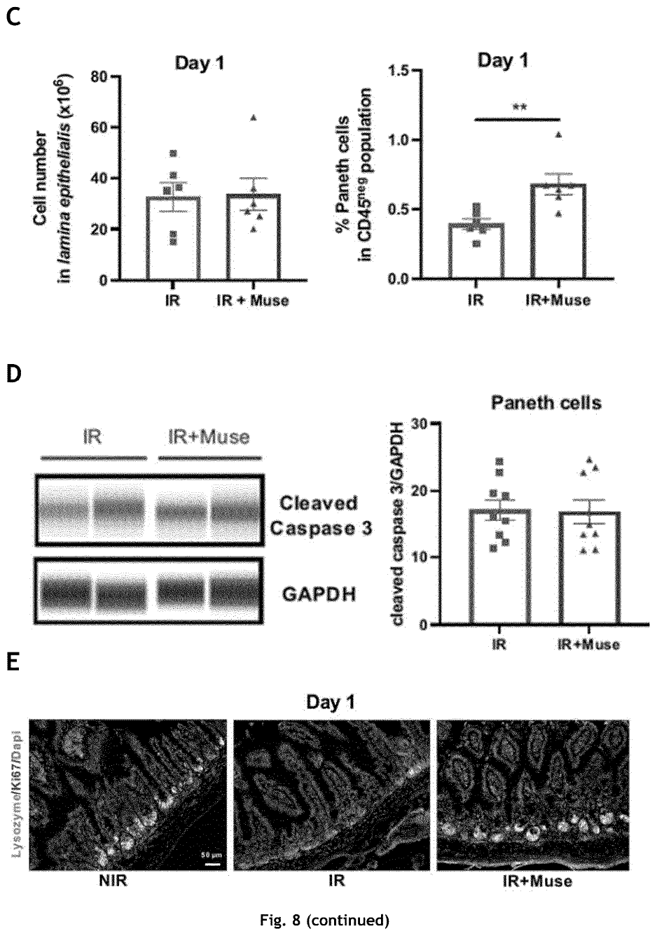

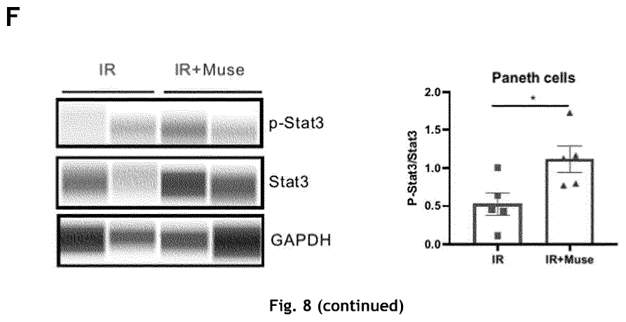

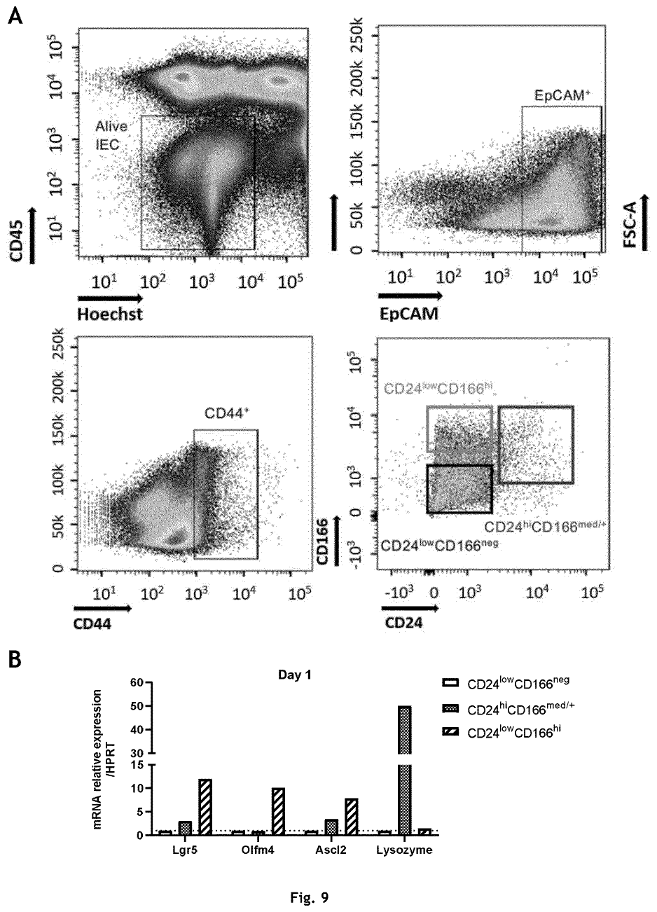

Previous reports indicate that several human tissues are readily accessible for Muse isolation, including adult tissues such as bone marrow (BM) and embryonic tissues such as Wharton's Jelly (WJ) of umbilical cord [17,18]. We first compared WJ-Muse derived from mesenchymal stem cells (MSC) isolated from WJ of umbilical cord by enzymatic digestion and BM-Muse obtained from marketed MSC to select the best source of Muse with a potential therapeutic use in GIS. Almost 100% of isolated WJ-MSC are viable, negative for the hematopoietic surface marker CD45 and positive for different mesenchymal surface markers such as CD44, CD105, CD90 and CD73 (FIG. 1A). Based on the Muse specific SSEA-3 marker, the mean percentage of Muse obtained after MSC isolation from Wharton's Jelly ranged from 1 to 4% while the mean percentage of Muse within BM-derived MSC was about 1% (FIG. 1A). To get a high number of cells, Muse were isolated by flow cytometry after in vitro amplification of WJ-MSC or BM-MSC using culture protocols previously described (FIG. 1B). In culture, WJ-MSC exhibited a higher proliferative capacity than BM-MSC, with higher cumulative cell population (FIG. 1B). After seven passages of culture of MSC, the mean percentage of WJ-Muse was 11%, while the mean percentage of BM-Muse was 8% (FIG. 2A and FIG. 1B). WJ- and BM-Muse share the same characteristics as MSC, (i) the plastic adherence potential and spindle shape fibroblast-like morphology when maintained in standard culture conditions and (ii) formed spontaneously clusters in cell suspension culture (FIG. 1C). Both WJ- and BM-Muse were negative for CD45 and positive for CD105 (FIG. 2C). WJ- and BM-Muse pluripotent potential was studied by RTqPCR. WJ-Muse expressed higher levels of Sox2, Nanog and Oct3/4 than BM-Muse suggesting a more immature status (FIG. 2D). Then, multilineage differentiation capacity of WJ- and BM-Muse was studied. WJ- and BM-Muse displayed the same ability to differentiate in adipocytes (positive staining of cytoplasmic lipid droplets with oil Red O) (FIG. 2E, top photos), in osteoclasts (positive expression of osteocalcin protein) (FIG. 2E, down photos) and in epithelial cells (expression of cytokeratin 18 (CK18) and Occludin (OCLN)) (FIG. 2E, lower panels). We then characterized the protein repertoires of WJ-Muse and BM-Muse using mass spectrometry (MS)-based quantitative proteomics as WJ- and BM-Muse might express different set of proteins which may influence their biological properties. Among the 6,058 reliably identified and quantified human proteins, 744 were differentially expressed, with 343 and 401 proteins found to be significantly more abundant in BM- and WJ-Muse respectively (FIG. 1D). Gene Ontology (GO) analysis highlighted common biological properties of BM- and WJ-Muse such as angiogenesis, cell adhesion, cell migration and response to drug (FIG. 1E). However, proteins enriched in BM-Muse are related to collagen, extracellular matrix, development of tissues, anti-oxidant activity and adaptive immunity, which are important for wound healing and indicated the potential of BM-Muse in tissue damage repair (FIG. 2F, left panel). In contrast, WJ-Muse present proteins involved in innate immune response, which is considered as the first line of host defence in tissue injury. Interestingly, WJ-Muse are also enriched in proteins that are essential for intestinal barrier function such as the ICAM-1 protein, known to regulate the homing and the immunomodulatory activity as observed in intestinal mucosal wound healing (FIG. 2F, right panel) [20].

Altogether, these results showed that WJ-Muse share similar biological characteristics with BM-Muse but present many advantages: i) a higher frequency, ii) a more immature status, iii) an expression of proteins that interplay with the innate immune response that can modulate the healing process and (iv) an expression of proteins involved in intestinal barrier regeneration. Thus, WJ-Muse were good candidates for cell therapy in the GIS.

### 2.2. Immunoreactivity of WJ-Muse

The Muse isolated from adult tissues have immunosuppressive capacities [21,22] but no data are currently available from foetal Muse. Thus, we investigated whether WJ-Muse might regulate an immune response by cell membrane expression and/or secretion of immunosuppressive factors. WJ-Muse constitutively expressed soluble HLA-G5 and expressed HLA-G1, HLA-DR and PD-L1 only when WJ-Muse are pre-activated (primed) with the IFNγ and TNFα proinflammatory cytokines (FIG. 3A). Compared to naive cells, these pre-activated WJ-Muse overexpressed immunosuppressive factors such as IDO (84-fold), Cox2 (14.5-fold), PD-L1 (8.8fold) and at a lower level TGFβ1 (1.8-fold (FIG. 3B). Then, we investigated whether WJ-Muse could regulate the CD3⁺T-cell responses. Allogeneic co-cultures of WJ-Muse with unstimulated human peripheral blood mononuclear cells (CTL hPBMC) showed no change in the frequency or proliferation of human CD3⁺T-cells (FIG. 3C, upper panels). Xenogeneic cocultures with unstimulated murine spleen lymphocytes (CTL mSL) did not modify the frequency and proliferation of murine CD3⁺T-cells (FIG. 3C, left and right lower panels). Finally, we studied if WJ-Muse could modulate the proliferation of concanavalin A-stimulated hPBMC or mSL. In allogeneic and xenogeneic conditions, the frequency of CD3⁺T-cells did not change in the presence of different concentrations of WJ-Muse (FIG. 3D, left and right upper panels), whereas CD3⁺T-cell proliferation was significantly reduced when co-cultured with 10,000 or 20,000 WJ-Muse compared to concanavalin A-stimulated hPBMC or mSL alone (FIG. 3D, left and right lower panels).

Finally, as the immunomodulatory capacities of WJ-Muse could extend to other immune cell populations, we studied the effect of WJ-Muse on macrophages. Murine bone marrow-derived macrophages (BMDM) were co-cultured in absence or presence of WJ-Muse and characterized for M1-(Nos2) or M2-(Arg1) macrophage markers. Co-culture of BMDM with WJ-Muse highly increased the expression of Arg1 (843-fold) and weakly increased the expression of Nos2 (35-fold) (FIG. 3E), indicating that WJ-Muse might drive macrophage polarization toward anti-inflammatory M2-like phenotype, known to be involved in tissue remodelling and repair [23,24].

Altogether, these results showed that WJ-Muse display immunosuppressive properties through cell contact and/or soluble mediators, which are expressed either constitutively or induced by inflammation. These immunosuppressive properties might regulate their integration in injured tissue after intravenous injection and lead to inhibition of inflammation responses and to stimulation of the regenerative process.

### 2.3. WJ-Muse Migrate in the Irradiated Intestine and Improve Survival, Prevent Weight Loss and Reduce Damages of the Small Intestine in a GIS Mouse Model

To investigate any therapeutic potential of WJ-Muse (called Muse) in the treatment of GIS, 50,000 Muse or 50,000 MSC were intravenously injected in C57BL/6 mice 4 hours after a 18 Gy-abdominal IR, a localized dose known to induce a lethal GIS within 7 to 10 days [26]. As Muse can migrate and home into injured tissues [27], we first monitored any migration and homing of Muse in the irradiated small intestine. Seven days after injection of GFP-Muse in irradiated mice, we observed GFP⁺ cells in the environment of intestinal crypts (FIG. 4A) indicating that Muse have integrated the injured tissue.

A survival study showed that 70% of Muse-treated mice survived until 29 days, while untreated or MSC-treated mice died within 10 days (80% and 100% respectively) (FIG. 4B, left panel). Whereas all mice began to lose weight with a nadir around 7-10 days, Muse-treated mice lost less weight and recovered a normal one 29 days after IR (FIG. 4B, right panel). To specify the time-window of animal management to impair the GIS after IR injury, Muse were injected 24 hours or 5 days after the 18 Gy-abdominal IR. Seventy percent of mice treated 24 hours after irradiation survived with a reduced weight loss (FIG. 4C, right panel), whereas all mice treated 5 days after irradiation died within 8 days (FIG. 4C, left panel), suggesting that the injection of Muse must should be delivered during the first 24 hours after IR injury. Towards a therapeutic use, we studied the effectiveness of cryopreserved Muse. More than 90% of Muse were alive after thawing and remained positive for SSEA-3 (FIG. 5A). We treated mice 4 h after abdominal IR with 50,000 cryopreserved Muse and showed the same effect as fresh Muse with a better survival and a reduction in weight loss compared to untreated mice (FIG. 5B). Thus, most of in vivo experiments were performed with cryopreserved Muse.

At different times post-IR, surviving mice were sacrificed and the small intestines were harvested for macroscopic and histological evaluation. GIS mice treated 4 h post-IR with 50,000 Muse displayed neither shortening, nor weight loss of the small intestine 7 days after IR (FIG. 4D). In

Muse-treated mice, 7 days post-IR, the crypt-villi structures of the small intestine were indistinguishable from that of non-irradiated mice, whereas from 3.5 days post-IR, the small intestine of untreated irradiated mice showed a persistent mucosal architecture destruction, including villous denudation and crypt atrophy (FIG. 4E). The ability of the intestinal epithelium to regenerate depends on the number of surviving stem cells 3.5 days after IR [3]. In accordance with the histological results, at 3.5 days after IR, Muse-treated mice had a 2fold increase of clonogenic crypt count compared to untreated mice (FIG. 4F).

Altogether, these results suggest that during GIS, Muse injection prevented the mouse lethality and maintained intestinal integrity with increased survival of clonogenic crypts.

### 2.4. Muse Promote Regeneration of the Intestinal Epithelium

High dose-IR disrupt the intestinal epithelial tight junctions and lead to the mucosal barrier dysfunction making it permeable to luminal content [28,29]. To characterize the intestinal permeability 7 days after IR, we quantified the 4-kDa Dextran conjugated with fluorescein isothiocyanate (FITC-Dextran) in the plasma 4 hours after gavage of mice. Abdominal IR of untreated mice increased the intestinal permeability compared to non-irradiated mice (26.1±4.9 μg/ml versus 1.8±0.2 μg/ml). In contrast, Muse treatment restored the permeability of mucosal barrier (13.4±2.4 μg/ml) (FIG. 6A). As the permeability was characterized by tight junction loss, we studied the intestinal integrity by immunohistochemical analyses using zonula occludens-1 (ZO-1) antibody [30]. In untreated irradiated mice, the decreased expression of ZO-1 protein 7 days post-IR indicated a tight junction loss, whereas Muse injection has maintained ZO-1 expression in the cytomembrane of epithelial cells along the villi, as observed in the control non-irradiated mice (FIG. 6B). The maintenance/regeneration of the epithelial barrier is also mediated by epithelial cell adhesion molecules such as EpCAM that modifies cell-cell contact adhesion strength and tissue plasticity and regulates cell proliferation and differentiation [32]. Seven days post-IR, EpCAM protein expression decreased in the small intestine of untreated mice whereas the EpCAM expression level in the small intestine of Muse-treated mice was similar to the non-irradiated mice (FIG. 6C). Finally, we studied the epithelium regeneration of the small intestine after Muse treatment by Ki67/CD24 double-staining, CD24 being a marker of both intestinal crypt stem cells and Paneth cells [33]. In non-irradiated mice, proliferating epithelial cells (Ki67⁺) were located in the middle of the crypts whereas they disappeared from the crypts after IR (FIG. 6D, left and middle panels). Muse injection resulted in regeneration of the epithelium via the formation of enlarged hyperproliferative clusters (FIG. 6D, right panel).

Altogether, these results show that, during GIS, Muse injection orchestrated the regenerative process of intestinal epithelium by promoting cellular junction, epithelial adhesion and proliferation of intestinal epithelium cells (IEC).

### 2.5. Muse Increase Monocyte Recruitment and Promote M2-Like Macrophage Polarization in the Irradiated Intestine

The intestinal crypt regeneration after radio-induced injury of small intestine requires the ISC microenvironment including monocytes/macrophages, Paneth cells and molecular mediators [6,7,9]. Macrophages are mostly replenished by high turnover from blood monocytes recruited to the sites of injury [34]. First, the protein level of Monocyte-Chemoattractant protein-1 (MCP1), which enhances the migration of monocytes into tissues during inflammation was studied on small intestine supernatant from untreated or Muse-treated GIS mice compared to non-irradiated mice. One day post-IR, MCP-1 protein level increased 2.4- and 9.5-fold in the untreated and Muse-treated mice respectively. Seven days post-IR, the MCP-1 protein level returned to normal only in Muse-treated mice (FIG. 7A). This transient higher increase of MCP1 protein in GIS Muse-treated mice was associated with a 2-fold increase in Ly6C^{hi} monocytes population in intestinal lamina propria from Muse-treated mice, but not in lamina epithelialis (FIG. 7B). Finally, Muse treatment led to an increased number of CD68⁺/CD206⁺double-stained M2-like macrophage population at day 7 post-IR, whereas CD68⁺macrophages from untreated mice did not express the CD206 marker (FIG. 7C), suggesting that Muse might favour macrophage polarization towards an anti-inflammatory M2 phenotype in injured small intestine.

Altogether, these results suggest that Muse injection could rapidly but transiently enhance MCP-1 production that is associated with an early recruitment of monocytes into the lamina propria. Seven days post-IR, Muse treatment is associated with the orientation of macrophages towards a M2 phenotype. These two features might contribute to intestinal tissue regeneration.

2.6. Muse Treatment Increases Paneth Cell Proliferation Associated with Activated IL-6/Stat3 Signalling Pathway

IL-6, another mediator of ISC microenvironment known for the survival of intestinal epithelial cells and the regulation of Paneth cell number [26,27] has been analysed after Muse injection in GIS model. One day after the 18 Gy-abdominal IR, murine IL-6 protein level was increased 2.6-fold in untreated mice and was 4-fold enhanced in Muse-treated mice compared to untreated ones (FIG. 8A). These increased expressions of IL-6 were transient as IL-6 protein levels returned to normal 7 days after IR (FIG. 8A). The higher level of IL-6 prompted us to investigate the capacity of Muse to regulate the pool of Paneth cells. A 2-fold decreased number of lysozyme-positive Paneth cells per crypt was found in untreated mice one day after IR but not in Muse-treated mice indicating a better maintenance of Paneth cell per crypt (FIG. 8B).

To further analyse how Muse act on Paneth cells, we isolated by flow cytometry a Paneth cell-enriched fraction from lamina epithelialis using gating strategy described in FIG. 9A and characterized by highly expression of Lysozyme (FIG. 9AB). One day after IR, whereas the lamina epithelialis total cell number was not different (FIG. 8C, left panel), the frequency of Paneth cells was 1.7-fold higher in Muse-treated mice than in untreated mice (FIG. 8C, right panel) indicating that Muse can inhibit the radio-induced apoptosis of Paneth cells and/or increase Paneth cell proliferation. One day post-IR, a similar expression level of cleaved caspase 3 protein between untreated and Muse-treated mice (FIG. 8D) indicated no effect of Muse on Paneth cell apoptosis. In contrast, Paneth cell proliferation was increased in Muse-treated mice (FIG. 8E). As IL-6 regulates proliferation of Paneth cells through Stat3 activation [24], Stat3 and phospho-Stat3 (p-Stat3) protein levels were quantified in a sorted population enriched in Paneth cells one day post-IR from untreated or Muse-treated mice. A 2-fold increase of the p-Stat3/Stat3 ratio was found in Muse-treated mice (FIG. 8F).

Altogether, these results demonstrated that in this GIS model Muse could activate the IL6/Stat3 signalling pathway in Paneth cells and increased their proliferation.

### 3. Discussion

The intestine is one of the most radiosensitive organs in the body and high doses of IR after accidental or therapeutic exposure lead to a GIS with severe intestinal damages, including loss of epithelial stem cells, and a high mortality rate. There is currently no effective treatment that prevents and/or reduces GIS.

The discovery of Muse has renewed interest in stem cell-based therapy application due to their higher efficient therapeutic potential with a lower cell administration than MSC (2.1×10⁵ cells/kg) [15]. Clinical trials have been performed by intravenous injection of donor derived Muse without HLA-matching and immunosuppressive treatment [15]. However, there is no data showing the involvement of Muse in regeneration of intestinal tissue after IR. Thus, no data describe Muse-based therapy for radiation-induced pathologies (such as in radioinduced GIS) or pathologies of the intestine.

The present work is the first demonstration of a therapeutic effect of foetal Muse in a preclinical model of radio-induced pathology.

In the present study, we showed that only 50,000 WJ-Muse are sufficient to obtain the same therapeutic benefit on GIS, making a major advantage of Muse over MSC/SVF cells.

Moreover, a rapid management of patient could occur thanks to an allogeneic engraftment with cryopreserved cells, that we showed to have the same therapeutic capacity than freshly isolated Muse.

Muse can be directly isolated from human bone marrow and human connective tissues as skin [37], adipose tissue and more recently from umbilical cord [39]. They are also collectable from MSC after amplification in culture. To date, the best known and the most commonly used sources of Muse are the adult bone marrow and the adipose tissue [40]. However, Muse collection from these two tissues requires invasive procedures. In contrast, the collect and isolation of birth-associated tissues including umbilical cord is easy and safe for both mother and child, and these foetal tissues are presently approved as a therapeutic source of stem cells [41]. Nevertheless, the therapeutic potential of Muse purified from umbilical cord matrix Wharton's jelly (WJ) is not currently documented. Here, we show that a large amount of Muse can be obtained after amplification of WJ-MSC, indicating a potential large-scale production of Muse for clinical trials. WJ- and BM-Muse shared the similar basic characteristics including expression of mesenchymal and pluripotent markers and differentiation potential. However, WJ-Muse have interesting specific properties suitable for the integration and homing of cells and therefore suitable for the intestinal regenerative process during GIS. Indeed, WJ-Muse expressed a higher level of the pluripotent markers Nanog, Oct3/4 and Sox2, characteristic of a more primitive status that limits a transplantation rejection [42]. They exhibit immunosuppressive properties and they expressed proteins implicated in intestinal barrier function such as ICAM-1 protein. All these characteristics contribute to the migration and homing of Muse to repair the injured tissue.

In our model, Muse migrate into irradiated small intestine and persist into the crypts. They promote the regeneration of intestinal epithelium characterized by (i) a hyperproliferation of crypt cells, (ii) an increased expression of the tight junction protein ZO-1 and (iii) an increased expression of adherent protein EpCAM. The epithelium regeneration after injury is dependent on ISC survival and on the response of surrounding microenvironment that constitutes the ISC niche. This niche is composed of multiple cells as Paneth cells and immune cells including macrophages, providing growth factors, cytokines, and ligands that modulate the survival, differentiation or proliferation of ISC in homeostatic condition and after injury. In our study, we show that WJ-Muse have a major role during the acute phase of response to IR, as Muse rapidly enhance the secretion of murine MCP-1 and IL-6 cytokines, promoting a protective immune response and a pro-regenerative microenvironment. After Muse injection, we show that the enhancement of murine MCP-1 secretion is associated with an increase in the recruitment of monocytes into the inflamed small intestine. Moreover, we observed a M2-like macrophage invasion of intestinal tissue 7 days post-IR, which could limit intestinal inflammatory damage and promote repair and tissue regeneration. Several studies support a key role of macrophages in crypt regeneration. Transplantation of bone marrow-derived adherent stromal cells depleted of all myeloid cells failed to reconstitute the irradiated ISC niche and ISC regeneration leading to death of GIS mice. Depletion of host macrophages with clodronate resulted in poor survival after IR [44,45]. In vitro, we demonstrated that Muse have the competence to modify the immunological reactions by limiting T-cell proliferation or by inducing polarization of macrophage into an anti-inflammatory M2-like phenotype. In fact, in vivo, Muse could challenge the inflammatory microenvironment generated after IR through, either their constitutive expression of factors such as HLA-G5, or their expression of factors such as IDO, Cox2, PD-L1 and TGFβ1 whose expression is under direct influence of the recipient's inflammatory status. HLA-G5 and PD-L1 are known to reduce inflammation and immune responses and to display tolerogenic properties through interactions with inhibitory receptors on immune cells [44,45]. IDO mediates the differentiation of monocytes into immunosuppressive M2 macrophages, which in turn contribute to T-cell suppression [48]. Prostaglandins synthesized from arachidonic acid by Cox2 suppressed radio-induced crypt apoptosis and enhanced crypt regeneration [49].

Our study showed likewise that Muse injection mediates a rapid increased level of murine IL6. In the small intestine, IL-6 has been shown to protect intestinal epithelial cells from apoptosis during prolonged inflammation [46,47]. Kuhn et al. showed the beneficial early IL-6 secretion in part produced by intra-epithelial lymphocytes for epithelial proliferation and intestinal wound healing after acute inflammatory injury [48]. Murine IL-6 production in Muse-treated mice during the acute inflammatory response is associated with an elevation of Paneth cell number one day post-IR, due to their highly enhanced proliferation through Stat3 signalling pathway activation. These findings are in agreement with previous data demonstrating that IL-6 is important for intestinal epithelial cell survival during acute inflammatory response after focal IR [49]. The IL-6/Stat3 signalling pathway regulates the Paneth cell number/proliferation through IL-6 receptor located on their basal membrane thereby increasing the proliferation of ISC [36].

Several studies showed that after radio-induced injury, Paneth cells participate actively to the intestinal tissue repair through their dedifferentiation into Lgr5⁺ISC [50,51] or facilitate ISC recovery by providing several niche factors such as wnt3a and metabolic intermediates [52-54]. Altogether, the results presented here reveal that Muse injection is a feasible strategy to rapidly manage GIS due to their high regenerative capacity with only few cells injected.

### BIBLIOGRAPHIC REFERENCES

1. François A, Milliat F, Guipaud O, Benderitter M. Inflammation and immunity in radiation damage to the gut mucosa. Biomed Res Int. 2013; 2013:123241.
2. Gelberg H B. Comparative anatomy, physiology, and mechanisms of disease production of the esophagus, stomach, and small intestine. Toxicol Pathol. 2014; 42:54-66.
3. Potten C S. Stem cells in gastrointestinal epithelium: numbers, characteristics and death. Philos Trans R Soc Lond B Biol Sci. 1998; 353:821-30.
4. Barker N, van Oudenaarden A, Clevers H. Identifying the stem cell of the intestinal crypt: strategies and pitfalls. Cell Stem Cell. 2012; 11:452-60.
5. Santos AJM, Lo Y-H, Mah A T, Kuo C J. The Intestinal Stem Cell Niche: Homeostasis and Adaptations. Trends in Cell Biology. 2018; 28:1062-78.
6. Biswas S, Davis H, Irshad S, Sandberg T, Worthley D, Leedham S. Microenvironmental control of stem cell fate in intestinal homeostasis and disease. The Journal of Pathology. 2015; 237:135-45.
7. Hageman J H, Heinz M C, Kretzschmar K, van der Vaart J, Clevers H, Snippert HJG. Intestinal Regeneration: Regulation by the Microenvironment. Dev Cell. 2020; 54:435-46.
8. Cray P, Sheahan B J, Dekaney C M. Secretory Sorcery: Paneth Cell Control of Intestinal Repair and Homeostasis. Cellular and Molecular Gastroenterology and Hepatology. Elsevier; 2021; 12:1239-50.
9. Saha S, Aranda E, Hayakawa Y, Bhanja P, Atay S, Brodin N P, et al. Macrophage-derived extracellular vesicle-packaged WNTs rescue intestinal stem cells and enhance survival after radiation injury. Nat Commun. Nature Publishing Group; 2016; 7:13096.
10. Bentzen S M. Preventing or reducing late side effects of radiation therapy: radiobiology meets molecular pathology. Nat Rev Cancer. 2006; 6:702-13.
11. Chen G, Han Y, Zhang H, Tu W and Zhang S. Radiotherapy-induced Digestive injury: Diagnosis, Treatment and Mechanisms. Frontiers in Oncology. 2021; 11:757973.
12. Dezawa M. Muse Cells Provide the Pluripotency of Mesenchymal Stem cells: Direct Contibution of Muse cells to tissue regeneration. Cell transplantation. 2016; 25:849-861.
13. Yabuki H, Wakao S, Kushida Y, Dezawa M, Okada Y. Human Multilineage-differentiating Stress-Enduring Cells Exert Pleiotropic Effects to Ameliorate Acute Lung IschemiaReperfusion Injury in a Rat Model. Cell Transplant. 2018; 27:979-93.
14. Yamada Y, Wakao S, Kushida Y, Minatoguchi S, Mikami A, Higashi K, et al. S1P-S1PR2 Axis Mediates Homing of Muse Cells Into Damaged Heart for Long-Lasting Tissue Repair and Functional Recovery After Acute Myocardial Infarction. Circ Res. 2018; 122:1069-83.
15. Noda T, Nishigaki K, Minatoguchi S. Safety and Efficacy of Human Muse Cell-Based Product for Acute Myocardial Infarction in a First-in-Human Trial. Circ J. 2020; 84:1189-92.
16. Fujita Y, Nohara T, Takashima S, Natsuga K, Adachi M, Yoshida K, et al. Intravenous allogeneic multilineage-differentiating stress-enduring cells in adults with dystrophic epidermolysis bullosa: a phase 1/2 open-label study. J Eur Acad Dermatol Venereol. 2021; 35: e528-31.
17. Leng Z, Sun D, Huang Z, Tadmori I, Chiang N, Kethidi N, et al. Quantitative Analysis of SSEA3⁺Cells from Human Umbilical Cord after Magnetic Sorting. Cell Transplant. 2019; 28:907-23.
18. Kuroda Y, Kitada M, Wakao S, Nishikawa K, Tanimura Y, Makinoshima H, et al. Unique multipotent cells in adult human mesenchymal cell populations. Proceedings of the National Academy of Sciences. Proceedings of the National Academy of Sciences; 2010; 107:8639-43.
19. Kuroda Y, Wakao S, Kitada M, Murakami T, Nojima M, Dezawa M. Isolation, culture and evaluation of multilineage-differentiating stress-enduring (Muse) cells. Nat Protoc. Nature Publishing Group; 2013; 8:1391-415.
20. Sumagin R, Brazil J C, Nava P, Nishio H, Alam A, Luissint A C, et al. Neutrophil interactions with epithelial-expressed ICAM-1 enhances intestinal mucosal wound healing. Mucosal Immunol. 2016; 9:1151-62.
21. Uchida H, Niizuma K, Kushida Y, Wakao S, Tominaga T, Borlongan C V, et al. Human Muse Cells Reconstruct Neuronal Circuitry in Subacute Lacunar Stroke Model. Stroke. 2017; 48:428-35.
22. Gimeno M L, Fuertes F, Barcala Tabarrozzi A E, Attorressi Al, Cucchiani R, Corrales L, et al. Pluripotent Nontumorigenic Adipose Tissue-Derived Muse Cells have Immunomodulatory Capacity Mediated by Transforming Growth Factor-β1. Stem Cells Transl Med. 2017; 6:161-73.
23. Silva ZEV da, Lehr H-A, Velin D. In vitro and in vivo Repair Activities of Undifferentiated and Classically and Alternatively Activated Macrophages. PAT. Karger Publishers; 2014; 81:86-93.
24. Lacave-Lapalun J-V, Benderitter M, Linard C. Flagellin or lipopolysaccharide treatment modified macrophage populations after colorectal radiation of rats. J Pharmacol Exp Ther. 2013; 346:75-85.
25. Xu H-H, Xie Y-Y, Jun-Gan null, Yang Z, Han Q-Y. Dynamic changes of soluble HLA-G and cytokine plasma levels in cervical cancer patients: potential role in cancer progression and immunotherapy. J Cancer Res Clin Oncol. 2022;
26. Bensemmane L, Squiban C, Demarquay C, Mathieu N, Benderitter M, Le Guen B, et al. The stromal vascular fraction mitigates radiation-induced gastrointestinal syndrome in mice. Stem Cell Res Ther. 2021; 12:309.
27. Cao J, Yang Z, Xiao R, Pan B. Regenerative potential of pluripotent nontumorgenetic stem cells: Multilineage differentiating stress enduring cells (Muse cells). Regen Ther. 2020; 15:92-6.
28. Shukla P K, Gangwar R, Manda B, Meena A S, Yadav N, Szabo E, et al. Rapid disruption of intestinal epithelial tight junction and barrier dysfunction by ionizing radiation in mouse colon in vivo: protection by N-acetyl-l-cysteine. Am J Physiol Gastrointest Liver Physiol. 2016; 310: G705-715.
29. Chelakkot C, Ghim J, Ryu S H. Mechanisms regulating intestinal barrier integrity and its pathological implications. Exp Mol Med. 2018; 50:1-9.
30. Kuo W-T, Zuo L, Odenwald M A, Madha S, Singh G, Gurniak C B, et al. The Tight Junction Protein ZO-1 Is Dispensable for Barrier Function but Critical for Effective Mucosal Repair. astroenterology. 2021; 161:1924-39.
31. Huang L, Yang Y, Yang F, Liu S, Zhu Z, Lei Z, et al. Functions of EpCAM in physiological processes and diseases (Review). Int J Mol Med. 2018; 42:1771-85.
32. Schnell U, Cirulli V, Giepmans BNG. EpCAM: Structure and function in health and disease. Biochimica et Biophysica Acta (BBA)-Biomembranes. 2013; 1828:1989-2001.
33. von Furstenberg R J, Gulati A S, Baxi A, Doherty J M, Stappenbeck T S, Gracz A D, et al. Sorting mouse jejunal epithelial cells with CD24 yields a population with characteristics of intestinal stem cells. Am J Physiol Gastrointest Liver Physiol. 2011; 300: G409-417.
34. Bain C C, Schridde A. Origin, Differentiation, and Function of Intestinal Macrophages. Frontiers in Immunology [Internet]. 2018 [cited 2022 Sep. 2];9. Available from:
   https://www.frontiersin.org/articles/10.3389/fimmu.2018.02733
35. Takada Y, Hisamatsu T, Kamada N, Kitazume M T, Honda H, Oshima Y, et al. Monocyte Chemoattractant Protein-1 Contributes to Gut Homeostasis and Intestinal Inflammation by Composition of IL-10-Producing Regulatory Macrophage Subset. J I. 2010; 184:2671-6.
36. Jeffery V, Goldson A J, Dainty J R, Chieppa M, Sobolewski A. IL-6 Signaling Regulates Small Intestinal Crypt Homeostasis. J Immunol. 2017; 199:304-11.
37. Gong W, Guo M, Han Z, Wang Y, Yang P, Xu C, et al. Mesenchymal stem cells stimulate intestinal stem cells to repair radiation-induced intestinal injury. Cell Death Dis. Nature Publishing Group; 2016; 7: e2387-e2387.
38. Galderisi U, Peluso G, Di Bernardo G. Clinical Trials Based on Mesenchymal Stromal Cells are Exponentially Increasing: Where are We in Recent Years? Stem Cell Rev Rep. 2022; 18:23-36.
39. Moll G, Alm J J, Davies L C, von Bahr L, Heldring N, Stenbeck-Funke L, et al. Do cryopreserved mesenchymal stromal cells display impaired immunomodulatory and therapeutic properties? Stem Cells. 2014; 32:2430-42.
40. Yang Y-H K, Ogando C R, Wang See C, Chang T-Y, Barabino G A. Changes in phenotype and differentiation potential of human mesenchymal stem cells aging in vitro. Stem Cell Research & Therapy. 2018; 9:131.
41. Chinnadurai R, Copland I B, Garcia M A, Petersen C T, Lewis C N, Waller E K, et al. Cryopreserved Mesenchymal Stromal Cells Are Susceptible to T-Cell Mediated Apoptosis Which Is Partly Rescued by IFNγ Licensing. Stem Cells. 2016; 34:2429-42.
42. Rostamzadeh A, Anjomshoa M, Kurd S, Chai J-K, Jahangiri F, Nilforoushzadeh M A, et al. The Role of Wharton's Jelly Mesenchymal Stem Cells in Skin Reconstruction. J Skin Stem Cell [Internet]. Brieflands; 2015 [cited 2022 Sep. 2];2. Available from:

https://brieflands.com/articles/jssc-60143.html #abstract
43. Contini P, Murdaca G, Puppo F, Negrini S. HLA-G Expressing Immune Cells in Immune Mediated Diseases. Front Immunol. 2020; 43:1613.
44. Riella L V, Paterson A M, Sharpe A H, Chandraker A. Role of the PD-1 pathway in the immune response. Am J Transplant. 2012; 12:2575-87.
45. Dann S M, Spehlmann M E, Hammond D C, limura M, Hase K, Choi L J, et al. IL-6-dependent mucosal protection prevents establishment of a microbial niche for attaching/effacing lesionforming enteric bacterial pathogens. J Immunol. 2008; 180:6816-26.
46. Grivennikov S, Karin E, Terzic J, Mucida D, Yu G-Y, Vallabhapurapu S, et al. IL-6 and Stat3 are required for survival of intestinal epithelial cells and development of colitis-associated cancer. Cancer Cell. 2009; 15:103-13.
47. Kuhn K A, Manieri N A, Liu T-C, Stappenbeck T S. IL-6 stimulates intestinal epithelial proliferation and repair after injury. PLOS One. 2014; 9: e114195.
48. Bell B I, Koduri S, Salas Salinas C, Monslow J, Puré E, Ben-Josef E, et al. Interleukin 6 Signaling Blockade Exacerbates Acute and Late Injury From Focal Intestinal Irradiation. Int J Radiat Oncol Biol Phys. 2019; 103:719-27.
49. Mei X, Gu M, Li M. Plasticity of Paneth cells and their ability to regulate intestinal stem cells. Stem Cell Res Ther. 2020; 11:349.
50. Yu S, Tong K, Zhao Y, Balasubramanian I, Yap G S, Ferraris R P, et al. Paneth Cell Multipotency Induced by Notch Activation following Injury. Cell Stem Cell. 2018; 23:46-59.e5.
51. Sato T, van Es J H, Snippert H J, Stange D E, Vries R G, van den Born M, et al. Paneth cells constitute the niche for Lgr5 stem cells in intestinal crypts. Nature. 2011; 469:415-8.
52. Schmitt M, Schewe M, Sacchetti A, Feijtel D, van de Geer W S, Teeuwssen M, et al. Paneth Cells Respond to Inflammation and Contribute to Tissue Regeneration by Acquiring Stem-like Features through SCF/c-Kit Signaling. Cell Reports. 2018; 24:2312-2328.e7.
53. Das S, Feng Q, Balasubramanian I, Lin X, Liu H, Pellón-Cardenas 0, et al. Colonic healing requires Wnt produced by epithelium as well as Tagln+ and Acta2⁺stromal cells. Development. 2022; 149: dev199587.
54. Peltzer J, Montespan F, Thepenier C, Boutin L, Uzan G, Rouas-Freiss N, et al.

Heterogeneous Functions of Perinatal Mesenchymal Stromal Cells Require a Preselection Before Their Banking for Clinical Use. Stem Cells and Development. Mary Ann Liebert, Inc., publishers; 2015; 24:329-44.
55. Casabona M G, Vandenbrouck Y, Attree I, Couté Y. Proteomic characterization of Pseudomonas aeruginosa PAO1 inner membrane. PROTEOMICS. 2013; 13:2419-23.
56. Bouyssié D, Hesse A-M, Mouton-Barbosa E, Rompais M, Macron C, Carapito C, et al. Proline: an efficient and user-friendly software suite for large-scale proteomics. Bioinformatics. 2020; 36:3148-55.
57. Wieczorek S, Combes F, Lazar C, Giai Gianetto Q, Gatto L, Dorffer A, et al. DAPAR & ProStaR: software to perform statistical analyses in quantitative discovery proteomics. Bioinformatics. 2017; 33:135-6.
58. Gallouet A-S, Ferri F, Petit V, Parcelier A, Lewandowski D, Gault N, et al. Macrophage production and activation are dependent on TRIM33. Oncotarget. 2016; 8:5111-22.
59. Gorin N—C, Fliedner™, Gourmelon P, Ganser A, Meineke V, Sirohi B, et al. Consensus conference on European preparedness for haematological and other medical management of mass radiation accidents. Ann Hematol. 2006; 85:671-9.
60. Zhang W, Hubbard A, Jones T, Racolta A, Bhaumik S, Cummins N, et al. Fully automated 5-plex fluorescent immunohistochemistry with tyramide signal amplification and same species antibodies. Lab Invest. Nature Publishing Group; 2017; 97:873-85.
61. Wang F, Scoville D, He X C, Mahe M M, Box A, Perry J M, et al. Isolation and Characterization of Intestinal Stem Cells Based on Surface Marker Combinations and Colony-Formation Assay. Gastroenterology. 2013; 145:383-395.e21.

1-16. (canceled)

17. A method for treating a radiation-induced gastrointestinal syndrome (GIS), comprising the administration of a therapeutically effective amount of Multilineage-differentiating stress enduring (Muse) cells, wherein the Muse cells are SSEA-3⁺CD105⁺, to a subject in need thereof.

18. The method according to claim 17, wherein the Muse cells are further characterized by a surface marker selected from:
CD44⁺;
CD73⁺;
CD90⁺;
CD45⁻; and
any combination thereof.

19. The method according to claim 17, wherein the Muse cells are obtained from adult or embryonic tissue, such as bone marrow, peripheral blood, adipose tissue, umbilical cord, or any mixture thereof.

20. The method according to claim 1, wherein the Muse cells express at least one gene selected from the group consisting of the genes encoding:
SRY-box transcription factor 2 (Sox2),
Nanog homeobox (Nanog),
Octamer-binding transcription factor3/4 (Oct3/4), and
Soluble human leukocyte antigen-G5 (HLA-G5).

21. The method according to claim 17, wherein the Muse cells express proteins active in cellular pathways selected from the group consisting of angiogenesis, cell adhesion, cell migration, response to drug, innate immune response, and intestinal barrier function.

22. The method according to claim 17, wherein the Muse cells are pluripotent.

23. The method according to claim 17, wherein the Muse cells reduce CD3⁺ T-cell proliferation in presence of concanavalin A-stimulated human peripheral blood mononuclear cells (hPBMC) or of concanavalin A-stimulated murine spleen lymphocytes (mSL).

24. The method according to claim 17, wherein the Muse cells increase the levels of expression, by macrophages, of the gene encoding Arginase 1 (Arg1).

25. The method according to claim 17, wherein the radiation is preferably selected from the group consisting of:
electromagnetic radiation;
particle radiation;
acoustic radiation;
gravitational radiation, that takes the form of gravitational waves, or ripples in the curvature of spacetime; and
any combination thereof.

26. A method for treating a radiation-induced gastrointestinal syndrome (GIS), comprising the administration of a therapeutically effective amount of a composition comprising the Muse cells according to claim 17 to a subject in need thereof.

27. The method according to claim 26, wherein the composition comprises an amount of at least 50,000 Muse cells.

28. The method according to claim 26, wherein the composition comprises an amount of 1,000,000 Muse cells or less.

29. The method according to claim 26, wherein the composition is formulated for an administration by injection.

30. The method according to claim 17, wherein the Muse cells migrate toward and integrate the tissue injured by said radiation-induced gastrointestinal syndrome (GIS) or migrate toward and integrate the intestine tissue damaged by said gastrointestinal syndrome (GIS).

31. The method according to claim 17, wherein in a subject in need thereof, administered with the Muse cells or the composition, any of:
a) survival rates increase;
b) weight loss reduces;
c) levels of expression of the gene encoding Monocyte-Chemoattractant protein-1 (MCP1) increase;
d) levels and/or percentages of Ly6C^{hi} monocytes population increases;
e) levels and/or percentages of CD68⁺CD206⁺ (M2-like) macrophages increase; and
f) any combination thereof.

32. The method according to claim 17, wherein in a subject in need thereof, administered with the Muse cells or the composition, any of:
a) the crypt-villi structures of the intestine tissue are repaired at least partly;
b) the clonogenic crypt count increases in the intestine tissue;
c) the permeability of mucosal barrier of the intestine tissue is repaired at least partly;
d) the levels of expression of the gene encoding Zonula occludens-1 (ZO-1) is restored in the cytomembrane of epithelial cells in the villi of the intestine tissue, at least partly;
e) the levels of expression of the gene encoding Epithelial cell adhesion molecule (EpCAM) by cells of the intestine tissue is restored at least partly;
f) the epithelium of the intestine tissue is repaired at least partly;
g) the levels and/or the percentages of intestinal crypt stem cells and Paneth cells increase in the intestine, and the levels and/or the percentages of Ki67⁺CD24⁺ cells in the intestine tissue preferably increases; and
h) the levels and/or the percentage of IL6 increases, and the levels and/or the percentages of Paneth cells preferably increase in the intestine tissue, and
i) any combination thereof.

33. The method according to claim 17, wherein the Muse cells are mammal Muse cells.

34. The method according to claim 17, wherein the Muse cells are human Muse cells.

35. The method according to claim 17, wherein the Muse cells are obtained from umbilical cord.

36. The method according to claim 17, wherein the Muse cells are obtained from Wharton's jelly of umbilical cord.

37. The method according to claim 17, wherein the Muse cells express proteins active in innate immune response or intestinal barrier function.

38. The method according to claim 17, wherein the Muse cells express proteins active in innate immune response and intestinal barrier function.

39. The method according to claim 17, wherein the Muse cells differentiate in any cell type selected from the group consisting of adipocytes, osteoclasts, and epithelial cells.

40. The method according to claim 17, wherein the Muse cells reduce CD3⁺T-cell proliferation in presence of concanavalin A-stimulated human peripheral blood mononuclear cells (hPBMC) and of concanavalin A-stimulated murine spleen lymphocytes (mSL).

41. The method according to claim 17, wherein the radiation is preferably selected from the group consisting of:
electromagnetic radiation selected from the group consisting of radio waves, microwaves, infrared, visible light, ultraviolet, x-rays, and gamma radiation (γ);
particle radiation selected from the group consisting of alpha radiation (a), beta radiation (B), proton radiation, and neutron radiation (particles of non-zero rest energy);
acoustic radiation, selected from the group consisting of ultrasound, sound, and seismic waves (dependent on a physical transmission medium);
gravitational radiation, that takes the form of gravitational waves, or ripples in the curvature of spacetime; and
any combination thereof.

42. The method according to claim 26, wherein the composition comprises an amount of Muse cells ranging from 50000 to 15 million Muse cells.

43. The method according to claim 26, wherein the composition comprises an amount of Muse cells ranging from 100000 to 12 million Muse cells.

44. The method according to claim 26, wherein the composition comprises an amount of Muse cells ranging from 500000 to 10 million Muse cells.

45. The method according to claim 26, wherein the composition comprises an amount of Muse cells ranging from 1 million to 5 million Muse cells.

46. The method according to claim 26, wherein the composition is formulated for an administration by injection via intravenous route.

47. The method according to claim 17, wherein the Muse cells repair at least partly the tissue damaged by said radiation-induced gastrointestinal syndrome (GIS) or the intestine tissue injured by said gastrointestinal syndrome (GIS) or maintain at least partly the integrity of the tissue exposed to the radiations or the intestine tissue affected by said gastrointestinal syndrome (GIS).

48. The method according to claim 17, wherein the Muse cells repair at least partly the tissue damaged by said radiation-induced gastrointestinal syndrome (GIS) or the intestine tissue injured by said gastrointestinal syndrome (GIS) and maintain at least partly the integrity of the tissue exposed to the radiations or the intestine tissue affected by said gastrointestinal syndrome (GIS).
